(19) United States
(12) Patent Application Publication
(10) Pub. No.: US 2026/0007686 A1
(43) Pub. Date: Jan. 8, 2026

(54) COMPOSITIONS AND METHODS FOR IMPROVING VISION

(71) Applicant: Ripple Therapeutics Corporation, Toronto (CA)

(72) Inventors: Ian Charles PARRAG, Mississauga, ON (CA); Wendy Alison NAIMARK, Toronto, ON (CA); Madeline Jenna SIMPSON, Toronto, ON (CA); Thomas Patrick REEVES, Toronto, ON (CA); Kelli-Anne Nicole WOOTTON, Toronto, ON (CA); Gillian Claire MACKEY, Toronto, ON (CA); Dimitra LOUKA, Toronto, ON (CA); Kyle Giovanni BATTISTON, Toronto, ON (CA); Hans Christian FISCHER, Toronto, ON (CA); Matthew Alexander John STATHAM, Milton, ON (CA)

(21) Appl. No.: 18/992,519

(22) PCT Filed: Jul. 12, 2023
(86) PCT No.: PCT/IB2023/000420

§ 371 Date: Jan. 8, 2025

Related U.S. Application Data
(60) Provisional application No. 63/368,223, filed on Jul. 12, 2022.
(60) Provisional application No. 63/378,809, filed on Oct. 7, 2022.

(51) Int. Cl. A61K 31/573; A61F 9/00; A61K 9/00; A61K 45/06; A61P 27/02

(52) U.S. Cl. CPC A61K 31/573; A61F 9/0017; A61K 9/0051; A61K 45/06; A61P 27/02

(57) ABSTRACT

Implants comprising steroid dimers for use in improving vision and treating ocular disorders and conditions are provided. Said steroid dimers have the structure D1-L-D2 with D1 and D2 preferably being dexamethasone with the dexamethasone radicals linked by a hydrolyzable linker L. The implants provide for a controlled release of the dexamethasone with any treatment potentially involving the use of multiple implants intravitreally administered no less than 1 month, preferably no less than 3 months after intravitreally administration of the prior implant. The implants may also be used in conjunction with other therapeutic agents including anti-inflammatory or angiogenesis factors. These implants have utility in improving visual acuity, treating vision deterioration and treating ocular disorders and conditions, in particular by decreasing central subfield thickness.

### CROSS-REFERENCE

This application claims the benefit of U.S. Provisional Application No. 63/368,223 filed Jul. 12, 2022, and U.S. Provisional Application No. 63/378,809 filed Oct. 7, 2022, which are each hereby incorporated by reference in their entirety herein.

### BACKGROUND OF THE INVENTION

Vision can be adversely affected by underlying ophthalmic conditions that prevent an eye from functioning properly. Such conditions can include, for example, ocular inflammation, post-operative surgery, and posterior ocular diseases or disorders, such as, macular edema (e.g., diabetic macular edema (DME)), retinal disorders (e.g., retinal vein occlusion (RVO)), and macular degeneration. While the current treatments for such conditions (e.g., DME and RVO) include compositions that can be implanted in an eye of an individual and release an active pharmaceutical ingredient (API) over a prolonged period of time, these treatments generally contain excipients, such as polymer carriers, that can create treatment challenges, such as, for example, burst release kinetics, low drug loading, and adverse inflammatory responses. Providing a therapeutic consisting entirely or almost entirely of API could address such challenges.

### SUMMARY OF THE INVENTION

Provided in some embodiments herein are compositions that preserve and/or improve the vision (e.g., vision acuity) of an individual (e.g., in need thereof). In some instances, the individual (e.g., in need thereof) has an underlying ophthalmic condition. In some instances, the individual (e.g., in need thereof) has a posterior ocular disease or disorder. In some instances, the individual in need thereof has a macular edema and/or a retinal disorder. In some instances, the individual in need thereof has diabetic macular edema (DME) or (e.g., macular edema secondary to) retinal vein occlusion (RVO). In some instances, a composition provided herein treats the underlying ophthalmic condition in the individual in need thereof. In some instances, treating the underlying ophthalmic condition in the individual in need thereof provides improvement and/or preservation of the vision (e.g., vision acuity) of the individual (e.g., in need thereof). In some instances, a composition provided herein treats a symptom or a secondary effect of an underlying ophthalmic condition in the individual in need thereof. In some instances, treating the symptom or the secondary effect of the underlying ophthalmic condition in the individual in need thereof provides improvement and/or preservation of the vision (e.g., vision acuity) of the individual (e.g., in need thereof).

In some instances, steroids used for ophthalmic use, such as those used as intravitreal implants in the clinic, provide undesirable effects, such as an increase in intraocular pressure (IOP) in an eye of an individual (e.g., after intravitreal implantation of a dexamethasone implant into the eye of the individual), especially after prolonged use. In some instances, such steroids or implants thereof increase IOP in the eye of the individual at both high and low doses. In some instances, a higher dose of such steroids or implants are used (e.g., provided that both the lower and higher doses increase IOP and the higher dose can be more efficacious than the lower dose).

Provided in some embodiments herein are implants (e.g., intravitreal implants), which comprise a high loading (e.g., 95% w/w or more) of a compound described herein (e.g., a steroid dimer (e.g., wherein each monomer of the steroid dimer is dexamethasone)), that do not (significantly) increase IOP of an eye of an individual, such as after intravitreal implantation into the eye of the individual. In some instances, the IOP in the eye of the individual receiving one or more implant (e.g., one or two implant) provided herein remains within (e.g., clinically) acceptable levels, such as even during prolonged use of the implant(s). In some instances, the IOP in the eye of the individual receiving one or more implant (e.g., one or two implant) provided herein does not increase above clinically acceptable levels, such as while the implant is present in the eye of the individual (e.g., for about six months or more). In some instances, the IOP of the eye of the individual receiving a lower dose of an implant provided herein (e.g., an implant of about 250 micrograms (μg) or less (w/w) of a compound described herein (e.g., Compound 1)) does not increase (e.g., above (clinically) acceptable levels).

In some instances, the IOP in the eye of the individual receiving one or more implant (e.g., one or two implant) provided herein even remains at (clinically) acceptable levels when a second implant, which is substantially identical to the first implant, is implanted into the eye of the individual. In some instances, the second implant is intravitreally implanted into the eye of the individual after the first implant has degraded (e.g., in the intraocular space of the eye of the individual). In some instances, at least some of the first implant remains in the eye of the individual when the second implant is intravitreally implanted into the (same) eye of the individual.

In some instances, administration of one or more implant provided herein into an eye of an individual (e.g., described herein) preserves and/or improves the vision (e.g., vision acuity) of the individual (e.g., the individual described herein). In some instances, administration of one or more implant provided herein into an eye of an individual (e.g., described herein) treats an ophthalmic disease or disorder (e.g., described herein) of the individual (e.g., the individual described herein).

In some instances, a composition (e.g., implant) provided herein comprises a compound having a first radical (D1) and a second radical (D2) (e.g., having the formula D1-L-D2). In some instances, D1 and D2 are each independently a steroid (radical). In some embodiments, D1 and D2 are each dexamethasone and L is a hydrolysable linker. In some embodiments, L is a hydrolyzable linker, such that when the compound of formula D1-L-D2 is administered (or when present in or otherwise exposed to an aqueous environment, such as a buffering solution, tears, vitreous humor, aqueous humor, serum, or the like), D1 and D2 are released (e.g., in their free, non-radical form). In some instances, such compounds are suitable for forming an article, implant, coating, or the like that consists essentially of the compound, such as containing about 10% or less of an excipient, about 5% or less of an excipient, about 1% or less of an excipient, or about 0.10% or less of an excipient. In some instances, the article, implant, coating, or the like described herein consists entirely or almost entirely (e.g., containing about 5% or less of an excipient, about 1% or less of an excipient, or about 0.1% or less of an excipient) of the compound. In some instances, such compounds are formed (e.g., as described herein) into controlled release delivery systems, such as delivery systems described herein (e.g., articles, implants, coatings, and the like). In some instances, such articles, implants, coatings, or the like are useful as controlled release delivery systems, such as providing dexamethasone to a treatment site (e.g., via intravitreal administration of an implant described herein in an eye of an individual in need thereof).

In some instances, provided herein is a composition (e.g., implant) that delivers to an eye of an individual in need thereof a therapeutically effective amount of (e.g., a free form of) a steroid, such as steroid described herein (e.g., dexamethasone). In some instances, the steroid (e.g., the free form of the steroid) is dexamethasone.

In some instances, a composition provide herein (e.g., an implant) releases an amount of dexamethasone sufficient to treat an underlying ophthalmic condition (a symptom or a secondary effect of an underlying ophthalmic condition) in an individual in need thereof for a (prolonged) period of time, such as for weeks, months, or years. In some instances, a composition provide herein (e.g., an implant) releases an amount of dexamethasone sufficient to improve and/or preserve the vision in an individual in need thereof for a (prolonged) period of time, such as for weeks, months, or years. In some instances, the improvement and/or maintenance of the vision of the individual in need thereof is perceived by the individual and/or measured, such as, by subjective methods (e.g., using a (ETDRS) letter chart, a Snellen chart, and/or a logMAR chart, such as to provide best corrected visual acuity (BCVA)) and/or quantitative measurements (e.g., measurements in central subfield thickness (CST)). In some instances, a composition provided herein releases an amount of dexamethasone sufficient to improve (e.g., increase) or maintain a level of vision acuity (e.g., as measured by BCVA) of an individual in need thereof for a (prolonged) period of time, such as for weeks, months, or years. In some instances, a composition provide herein releases an amount of dexamethasone sufficient to improve (e.g., decrease) or maintain a level of CST in an individual in need thereof for a (prolonged) period of time, such as for weeks, months, or years.

Provided in some embodiments herein is a method for improving vision (e.g., visual acuity) in an individual in need thereof, the method comprising (e.g., intravitreally) administering to an eye of the individual in need thereof one or more implant, each implant comprising a compound having a structure represented by Formula (I):

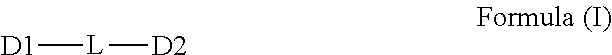

or a pharmaceutically acceptable salt or solvate thereof,
wherein,
   D1 and D2 are each independently a steroid radical (e.g., a dexamethasone radical); and
   L is a (e.g., hydrolyzable) linker, and
wherein the one or more implant collectively comprises an amount of the compound sufficient to improve vision in the eye of the individual in need thereof.

In some embodiments, the vision (e.g., the visual acuity) of the individual improves within about two months (e.g., within about one month, within about two weeks, or within about one week) of intravitreally administering the implant to the eye of the individual in need thereof.

In some embodiments, an improvement in vision is measured by a first (e.g., baseline) visual acuity measurement (e.g., from an ETDRS letter test) and a second (e.g., subsequent) visual acuity measurement (e.g., from an ETDRS letter test) (e.g., wherein the second visual acuity measurement increases compared to the first (e.g., baseline) visual acuity measurement). In some embodiments, the second visual acuity measurement is obtained within about 12 months (e.g., within about 11 months, within about 10 months, within about 9 months, within about 8 months, within about 7 months, within about 6 months, within about 5 months, within about 4 months, within about 3 months, within about 2 months, within about 1 month) of the first visual acuity measurement.

In some instances, one or more implant described herein is administered to the individual and the individual has no (substantial) change in vision (e.g., visual acuity) over a certain period of time, such as between a first (e.g., baseline) visual acuity measurement (e.g., from an ETDRS letter test) and a second (e.g., subsequent) visual acuity measurement. In some instances, one or more implant described herein is administered to the individual and the individual has a decrease in vision (e.g., visual acuity) over a certain period of time, such as between a first (e.g., baseline) visual acuity measurement (e.g., from an ETDRS letter test) and a second (e.g., subsequent) visual acuity measurement (e.g., from an ETDRS letter test). In some instances, one or more implant described herein is administered to the individual and the individual has a (substantial) improvement in vision (e.g., visual acuity) over a certain period of time, such as between a first (e.g., baseline) visual acuity measurement (e.g., from an ETDRS letter test) and a third (e.g., subsequent) visual acuity measurement. In some instances, one or more implant described herein is administered to the individual and the individual has an overall improvement in vision (e.g., visual acuity) during a course of treatment, such as over a period of about 6 months or more. In some instances, one or more implant described herein is administered to the individual and the vision (e.g., visual acuity) of the individual remains substantially the same during a course of treatment, such as while the compound is released from the implant in the eye of the individual.

In some embodiments, the first visual acuity measurement and the second visual acuity measurement are measured by Best Corrected Visual Acuity (BCVA).

In some embodiments, the vision of the individual (e.g., as measured by one or more ETDRS letter test) is improved by at least 5 letters (e.g., 5 or more letters, 6 or more letters, 7 or more letters, 8 or more letters, 9 or more letters, 10 or more letters, 11 or more letters, 15 or more letters, 20 or more letters, 25 or more letters, or 30 or more letters) by BCVA.

In some embodiments, the vision of the individual remains improved (e.g., a second visual acuity measurement remains higher than a first (e.g., baseline) visual acuity measurement) for at least about 2 months (e.g., about 2 months or more, about 3 months or more, about 4 months or more, about 5 months or more, about 6 months or more, about 7 months or more, about 8 months or more, about 9 months or more, about 10 months or more, about 11 months or more, or about 12 months or more) after intravitreally administering the implant to the eye of the individual in need thereof.

In some embodiments, the improvement in vision of the individual does not (substantially) decrease (e.g., a third visual acuity measurement remains higher than a second (e.g., prior) visual acuity measurement) during a course of treatment, such as over a period of at least two months or more.

In some embodiments, the improvement in vision of the individual continues to increase (e.g., a third visual acuity measurement remains higher than a second (e.g., prior) visual acuity measurement) during a course of treatment, such as over a period of about 6 months or more.

Provided in some embodiments herein is a method for treating vision deterioration in an individual in need thereof, the method comprising (e.g., intravitreally) administering to an eye of the individual in need thereof one or more implant, each implant comprising a compound having a structure represented by Formula (I):

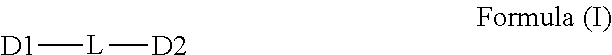

or a pharmaceutically acceptable salt or solvate thereof,
wherein,
   D1 and D2 are each independently a steroid radical (e.g., a dexamethasone radical); and
   L is a (e.g., hydrolyzable) linker, and
wherein the one or more implant collectively comprises an amount of the compound sufficient to treat the vision deterioration in the eye of the individual in need thereof.

In some embodiments, treating vision deterioration comprises reducing a rate of vision deterioration, maintaining a (current) level of vision, and/or improving vision in the individual in need thereof.

Provided in some embodiments herein is method for treating an ocular disorder or condition in an individual in need thereof, the method comprising (e.g., intravitreally) administering to an eye of the individual in need thereof a first implant and a second implant, the second implant being administered no less than one month (e.g., one month or more, two months or more, three months or more, four months or more, five months or more, six months or more, seven months or more, eight months or more, nine months or more, ten months or more, eleven months, or more, or twelve months or more) after the first implant, each implant comprising a compound having a structure represented by Formula (I):

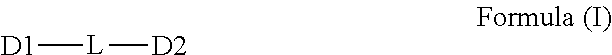

or a pharmaceutically acceptable salt or solvate thereof,
wherein,
   D1 and D2 are each independently a steroid radical (e.g., a dexamethasone radical); and
   L is a (e.g., hydrolyzable) linker, and
wherein the one or more implant collectively comprises an amount of the compound sufficient to treat the ocular disorder or condition in the eye of the individual in need thereof.

In some embodiments, a third implant is administered to the eye of the individual in need thereof. In some embodiments, the third implant is administered to the eye of the individual in need thereof about six months or more after the first implant is administered to the eye of the individual in need thereof. In some embodiments, the third implant is administered to the eye of the individual in need thereof about three months or more after the second implant is administered to the eye of the individual in need thereof. In some embodiments, the third implant is administered to the eye of the individual in need thereof about six months or more after the second implant is administered to the eye of the individual in need thereof.

In some embodiments, the second implant is administered within about three months of the first implant being administered to the eye of the individual in need thereof. In some embodiments, the second implant is administered to the eye of the individual in need thereof at least six months after the first implant is administered to the eye of the individual in need thereof. In some embodiments, the second implant is administered to the eye of the individual in need thereof no more than six months after the first implant is administered to the eye of the individual in need thereof (e.g., wherein the individual is lacking a response to the first implant, such as, lacking a decrease in CST of 5% or more compared to baseline, such as at or before month 3).

In some embodiments, an individual (of the population of individuals) described herein, such as administered one or more implant described herein (e.g., a Compound 1 IVT implant), does not require an (additional) intervention, retreatment, or rescue therapy. In some embodiments, an individual (of the population of individuals) described herein, such as administered one or more implant described herein (e.g., a Compound 1 IVT implant), does not require an IOP-lowering medication. In some embodiments, an individual (of the population of individuals) described herein, such as administered one or more implant described herein (e.g., a Compound 1 IVT implant), does not require a procedural intervention. In some embodiments, an individual (of the population of individuals) described herein, such as administered one or more implant described herein (e.g., a Compound 1 IVT implant), does not require a surgical intervention.

Provided in some embodiments herein is method for treating an ocular disorder or condition in an individual in need thereof, the method comprising (e.g., intravitreally) administering to an eye of the individual in need thereof one or more implant no more frequently than once every three months, each implant comprising a compound having a structure represented by Formula (I):

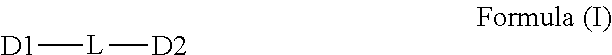

or a pharmaceutically acceptable salt or solvate thereof,
wherein,
   D1 and D2 are each independently a steroid radical (e.g., a dexamethasone radical); and
   L is a (e.g., hydrolyzable) linker, and
wherein the one or more implant collectively comprises an amount of the compound sufficient to treat the ocular disorder or condition in the eye of the individual in need thereof.

Provided in some embodiments herein is a method for treating an ocular disorder or condition in an individual in need thereof, the method comprising:
(i) administering (e.g., intravitreally) to an eye of the individual in need thereof a (pharmaceutical composition (e.g., an injection or an eye drop) comprising a) first agent (e.g., an anti-inflammatory agent and/or an angiogenesis inhibitor, such as an anti-growth factor and/or an anti-angiogenic factor); and
(ii) administering (e.g., intravitreally) to the eye of the individual in need thereof one or more implant comprising a compound having a structure represented by Formula (I):

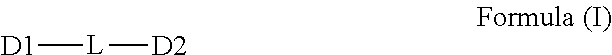

or a pharmaceutically acceptable salt or solvate thereof,
wherein,
   D1 and D2 are each independently a steroid radical (e.g., a dexamethasone radical); and
   L is a (e.g., hydrolyzable) linker.

In some embodiments, a method described herein further comprises injecting an (e.g., intravitreal (IVT)) injection comprising the first agent into the eye of the individual in need thereof.

In some embodiments, the first agent and the one or more implant are administered to the eye of the individual sequentially.

In some embodiments, the one or more implant is administered to the eye of the individual (e.g., at least one week) after the first agent is administered to the eye of the individual. In some embodiments, the one or more implant is administered to the eye of the individual after a washout period of the first agent, such as after a period of about one week or more (e.g., about one or two months).

In some embodiments, the first agent is formulated as an injection. In some embodiments, the first agent is formulated as an intravitreal injection. In some embodiments, the first agent is formulated as an intravitreal anti-inflammatory agent injection or an intravitreal angiogenesis inhibitor injection. In some embodiments, the first agent is formulated as an intravitreal steroid injection or an intravitreal anti-VEGF injection. In some embodiments, the first agent is formulated as an intravitreal steroid injection, such as an intravitreal dexamethasone implant described herein (e.g., Implant A). In some embodiments, the first agent is formulated as an intravitreal anti-VEGF injection. In some embodiments, the first agent is formulated as an eye drop.

In some embodiments, the first agent is used to treat and/or improve the symptoms of macular edema (e.g., diabetic macular edema (DME)). In some embodiments, the first agent is used to treat and/or improve the symptoms of retinal vein occlusion (RVO).

In some embodiments, the first agent is an angiogenesis inhibitor (e.g., a compound that prevents proliferation and/or formation of blood vessels). In some embodiments, the first agent is an anti-growth factor and/or an anti-angiogenic factor. In some embodiments, the first agent is an anti-growth factor. In some embodiments, the first agent is an anti-angiogenic factor. In some embodiments, the first agent is an anti-growth factor and an anti-angiogenic factor.

In some embodiments, the first agent is a vascular endothelial growth factor (VEGF) treatment (e.g., an anti-VEGF treatment), such as an anti-VEGF injection. In some embodiments, the first agent is a VEGF-targeting antibody, a VEGF antibody or a fragment thereof. In some embodiments, the first agent is a VEGF inhibitor. In some embodiments, the first agent is a VEGF antagonist. In some embodiments, the first agent is an anti-vascular endothelial growth factor-A (anti-VEGF-A).

In some embodiments, the one or more implant is administered after the first agent to extend treatment in the eye of the individual, such as for about one month or more (e.g., 6 months or more or 12 months or more). In some embodiments, the one or more implant is administered after the first agent to extend a reduction of symptoms, improvement in visual acuity (e.g., as measured by BCVA), and/or reduction in CST in the one or more eye of the individual.

In some embodiments, the ocular disorder or condition is a posterior ocular disease or disorder. In some embodiments, the ocular disorder or condition is a macular edema. In some embodiments, the ocular disorder or condition is a retinal disorder. In some embodiments, the ocular disorder or condition is diabetic macular edema (DME), macular edema secondary to retinal vein occlusion (RVO), or uveitis. In some embodiments, the ocular disorder or condition is DME or RVO. In some embodiments, the ocular disorder or condition is DME. In some embodiments, the ocular disorder or condition is RVO (e.g., central RVO).

In some embodiments, the implant is administered to the individual in need thereof once every three months or less, once every 4 months or less, once every 5 months or less, or once every 6 months or less. In some embodiments, the implant is administered to the individual in need thereof once every three to twelve months (e.g., once every six months).

Provided in some embodiments herein is a method for decreasing central subfield thickness (CST) in an individual in need thereof, the method comprising (e.g., intravitreally) administering to an eye of the individual in need thereof one or more implant, each implant comprising a compound having a structure represented by Formula (I):

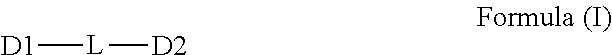

or a pharmaceutically acceptable salt or solvate thereof,
wherein,
   D1 and D2 are each independently a steroid radical (e.g., a dexamethasone radical); and
   L is a (e.g., hydrolyzable) linker, and
wherein the one or more implant collectively comprises an amount of the compound sufficient to decrease CST in the eye of the individual in need thereof.

In some embodiments, either or both of D1 or D2 are attached to L through an oxygen radical of the D1 or D2 (e.g., thereby forming a C—O bond).

In some embodiments, CST in the eye of the individual in need thereof decreases by at least 50 micrometers (μm) (e.g., 50 μm or more, 100 μm or more, 150 μm or more, or 200 μm or more), such as within about one month (e.g., within about one week) of intravitreally administering the implant to the eye of the individual in need thereof. In some embodiments, CST in the eye of the individual in need thereof decreases by at least 200 μm over an extended period of time, such as for a month or more (e.g., 6 months or more) after intravitreally administering the implant to the eye of the individual in need thereof.

In some embodiments, CST in the eye of the individual in need thereof decreases by at least 10% (e.g., 10% or more, 15% or more, 20% or more, 25% or more, 30% or more, 35% or more, or 40% or more) of the baseline CST, such as over an extended period of time, such as for a month or more (e.g., 6 months or more) after intravitreally administering the implant to the eye of the individual in need thereof.

In some embodiments, a decrease in CST is measured by a first (e.g., baseline) CST measurement and a second (e.g., subsequent) CST measurement (e.g., wherein the second CST measurement decreases compared to the first (e.g., baseline) CST measurement).

In some embodiments, CST in the eye of the individual decreases within about three months (e.g., within about two months, within about one month, within about two weeks, or within about one week) of intravitreally administering the implant to the eye of the individual in need thereof.

In some embodiments, CST of the individual remains decreased (e.g., a second CST measurement remains lower than a first (e.g., baseline) CST measurement) for at least about 2 months (e.g., about 2 months or more, about 3 months or more, about 4 months or more, about 5 months or more, about 6 months or more, about 7 months or more, about 8 months or more, about 9 months or more, about 10 months or more, about 11 months or more, or about 12 months or more) after intravitreally administering the implant to the eye of the individual in need thereof.

In some embodiments, the CST of the individual does not (substantially) increase (e.g., a third CST measurement remains lower than a second (e.g., prior) CST measurement) during a course of treatment, such as over a period of at least three months or more.

In some embodiments, the CST of the individual continues to decrease (e.g., a third CST measurement remains lower than a second (e.g., prior) CST measurement) during a course of treatment, such as over a period of about 6 months or more.

Provided in some embodiments herein, one or more implant is (e.g., intravitreally) administered to one or more eye of each individual of a population of individuals.

In some embodiments, the population of individuals have a mean change in CST that decreases (e.g., a second mean change in CST measurement for the population at a certain timepoint (e.g., at one-month of treatment) decreases compared to a first (mean change in) CST measurement for the population at another timepoint (e.g., baseline)), such as over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

In some embodiments, the mean change in CST decreases by about 50 μm or more (e.g., by about 100 μm or more, about 150 μm or more, about 200 μm or more, about 250 μm or more), such as over an extended period of time, such as for a month or more (e.g., 6 months or more) after intravitreally administering the implant to the eye of the individual in need thereof.

In some embodiments, the mean change in CST decreases by at least about 10% (e.g., about 10% or more, about 15% or more, about 20% or more, about 25% or more, about 30% or more, about 35% or more, about 40% or more, about 50% or more, about 60% or more, about 70% or more, or about 80% or more), such as compared to the (mean) baseline CST over an extended period of time (e.g., for a month or more (e.g., 6 months or more) after intravitreally administering the implant to the eye of the individual in need thereof).

Provided in some embodiments herein, a population of individuals with diabetic macular edema (DME) is administered two implants and the population has a mean change in CST that decreases, such as by at least about 60 μm or more (e.g., about 80 μm or more, about 100 μm or more, about 120 μm or more), over an extended period of time, such as over a period of at least about 1 month or more (e.g., about 2 months or more, about 3 months or more, about 4 months or more, about 5 months or more)

In some embodiments, a population of individuals with diabetic macular edema (DME) is administered one implant and the population has a mean change in CST that decreases, such as by at least about 40 μm or more (e.g., about 60 μm or more, about 80 μm or more, about 100 μm or more), over an extended period of time, such as over a period of at least about 1 month or more (e.g., about 2 months or more, about 3 months or more, about 4 months or more, about 5 months or more).

In some embodiments, a population of individuals with retinal vein occlusion (RVO) is administered two implants and the population has a mean change in CST that decreases, such as by at least about 100 μm or more (e.g., about 150 μm or more, about 200 μm or more), over an extended period of time, such as over a period of at least about 1 month or more (e.g., about 2 months or more, about 3 months or more, about 4 months or more, about 5 months or more, about 6 months or more).

In some embodiments, a population of individuals with retinal vein occlusion (RVO) is administered one implant and the population has a mean change in CST that decreases, such as by at least about 150 μm or more (e.g., about 200 μm or more, about 250 μm or more), over an extended period of time, such as over a period of at least about 1 month or more (e.g., about 2 months or more, about 3 months or more, about 4 months or more, about 5 months or more, about 6 months or more).

In some embodiments, the vision of the population of individuals (e.g., as measured by one or more ETDRS letter test) improves, such as by at least 5 letters (e.g., 5 or more letters, 6 or more letters, 7 or more letters, 8 or more letters, 9 or more letters) by BCVA.

In some embodiments, a population of individuals with RVO or DME is administered one or two implants and the population has a mean change in vision (improvement) of at least 5 letters by BCVA, such as over a period of at least about 1 month or more (e.g., about 2 months or more, about 3 months or more, about 4 months or more, about 5 months or more, about 6 months or more, about 7 months or more, about 8 months or more, about 9 months or more, about 10 months or more, about 11 months or more, about 12 months or more)

Provided in some embodiments herein is a composition comprising the compound described herein in an amount of about 250 micrograms (μg) or less, wherein the composition comprises at least 90% w/w of the compound.

Provided in some embodiments herein is an implant comprising the compound described herein in an amount of about 250 micrograms (μg) or less, wherein the implant comprises at least 90% w/w of the compound.

Provided in some embodiments herein is a dosage form comprising the compound described herein in an amount of about 250 micrograms (μg) or less, wherein the dosage form comprises at least 90% w/w of the compound.

In some embodiments, the compound has a structure represented by Formula (I-A):

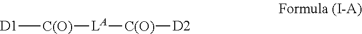

or a pharmaceutically acceptable salt or solvate thereof,
wherein,
   D1 and D2 are each independently a steroid radical (e.g., a dexamethasone radical); and
   L^{A} is substituted or un substituted alkyl or substituted or unsubstituted heteroalkyl (e.g., comprising one or more ethyleneglycol unit).

In some embodiments, an oxygen radical of the D1 and an oxygen radical of the D2 are each independently attached to a C(O) (e.g., the C(O) of Formula (I-A) is attached to D1 and D2, respectively).

In some embodiments, L^{A} is unsubstituted heteroalkyl.

In some embodiments, L^{A} is —O(CH₂CH₂O)ₙ, where n is an integer of 1-10. In some embodiments, n is an integer of 3-5. In some embodiments, n is 3.

In some embodiments, D1 and D2 are each represented by the same steroid radical structure. In some embodiments, D1 and D2 are each a dexamethasone radical (e.g., each independently being connected to the linker (e.g., L or LA) by an oxygen radical, thereby forming a C—O bond).

In some embodiments, the compound has a structure represented by:

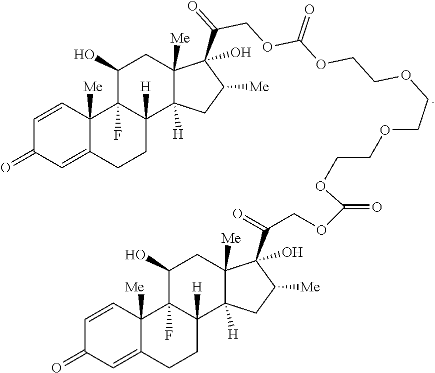

In some embodiments, the (one or more) implant comprises no more than about 500 micrograms (μg) of the compound. In some embodiments, the (one or more) implant comprises about 10 μg or more of the compound. In some embodiments, the (one or more) implant comprises about 10 μg to about 500 μg of the compound.

In some embodiments, the (one or more) implant comprises about 250 μg or less of the compound. In some embodiments, the (one or more) implant comprises no more than about 175 μg of the compound. In some embodiments, the (one or more) implant comprises about 156 μg or less of the compound. In some embodiments, the (one or more) implant comprises about 50 μg or more of the compound. In some embodiments, the (one or more) implant comprises about 75 μg or more of the compound. In some embodiments, the (one or more) implant comprises about 50 μg to about 250 μg of the compound. In some embodiments, the (one or more) implant comprises about 75 μg to about 175 μg of the compound. In some embodiments, the (one or more) implant comprises about 50 μg to about 150 μg of the compound. In some embodiments, the (one or more) implant comprises about 78 μg to about 106 μg of the compound.

In some embodiments, intraocular pressure (IOP) of the eye of the individual in need thereof does not increase above a clinically acceptable level (e.g., while the compound is released from the implant in the eye of the individual).

In some embodiments, IOP of the eye of the individual in need thereof does not increase above a level of about 25 millimeters of mercury (mmHg) (e.g., while the compound is released from the implant in the eye of the individual).

In some embodiments, intraocular pressure (IOP) of the eye of the individual in need thereof remains at a clinically acceptable level (e.g., while the compound is released from the implant in the eye of the individual). In some embodiments, IOP of the eye of the individual in need thereof remains below a level of about 25 mmHg (e.g., while the compound is released from the implant in the eye of the individual).

In some embodiments, the IOP of each individual of a population of individuals described herein is measured at a timepoint before (e.g., a baseline measurement) and at various timepoints after an implant described herein is administered to an individual of the population of individuals.

In some embodiments, the baseline measurement is measured before the individual is administered an implant described herein. In some embodiments, the baseline measurement is measured in an eye of the individual that an implant described herein was not administered, such as a fellow eye. In some embodiments, a change in IOP in an individual is determined by measuring the difference between a baseline IOP measurement and a IOP measurement measured after an implant described herein is administered to the individual. In some embodiments, the IOP measurement is measured at least one week after an implant described herein is administered to the individual. In some embodiments, the IOP measurement is measured at month one (M1), month two (M2), month three (M3), month four (M4), month five (M5), month six (M6), month seven (M7), month eight (M8), month nine (M9), month ten (M10), month eleven (M11), or month twelve (M12) of treatment. In some embodiments, a mean change in IOP of the population of individuals is determined by averaging each change in IOP of each individual at the respective timepoint. For example, a mean change in IOP at month 1 can be determined by averaging each change in IOP of each individual at M1, and so on until a mean change in IOP at each month of treatment is determined.

In some embodiments, the IOP of an individual of the population of individuals (or the entire population of individuals) remains substantially the same (e.g., the change in IOP does not change (i.e., increase or decrease) by more than about 15 millimeters of mercury (mmHg)) as compared to a baseline measurement. In some embodiments, the IOP of an individual of the population of individuals (or the entire population of individuals) remains substantially the same as a baseline measurement while the compound is released from the one or more implant (in the eye(s) of the individual(s)). In some embodiments, the IOP of an individual of the population of individuals (or the entire population of individuals) remains substantially the same as a baseline measurement during a course of treatment. In some embodiments, the IOP of an individual of the population of individuals (or the entire population of individuals) remains below a level of 35 mmHg during a course of treatment. In some embodiments, the IOP of an individual of the population of individuals (or the entire population of individuals) remains below a level of 25 mmHg during a course of treatment.

In some embodiments, the population of individuals have a change in IOP of no more than about 15 millimeters of mercury (mmHg) (e.g., while the compound is released from the one or more implant in the one or more eye), such as compared to a baseline measurement and/or over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

In some embodiments, a population of individuals with RVO is administered one or two implants and the population of individuals have a change in IOP of no more than about 10 mmHg (e.g., while the compound is released from the one or more implant in the one or more eye), such as compared to a baseline measurement and/or over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

In some embodiments, a population of individuals with DME is administered one or two implants and the population of individuals have a change in IOP of no more than about 5 mmHg (e.g., while the compound is released from the one or more implant in the one or more eye), such as compared to a baseline measurement and/or over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

In some embodiments, IOP of an eye of an individual of the population of individuals does not increase above a level of about 35 mmHg (e.g., while the compound is released from the implant in the eye of the individual).

In some embodiments, the amount of the compound administered to the individual in need thereof is sufficient to improve vision, treat vision deterioration, treat an ocular disorder or condition, and/or decrease CST in the eye of the individual in need thereof while avoiding an increase (e.g., a clinically significant increase) in intraocular pressure (IOP) in the individual in need thereof.

In some embodiments, the amount of the compound sufficient to improve vision, treat vision deterioration, treat an ocular disorder or condition, and/or decrease CST in the eye of the individual in need thereof is less than about 500 micrograms (μg) (e.g., 500 μg or less, 400 μg or less, 300 μg or less, 200 μg or less, 100 μg or less, or 50 μg or less). In some embodiments, the amount of the compound sufficient to improve vision, treat vision deterioration, treat an ocular disorder or condition, and/or decrease CST in the eye of the individual in need thereof is less than about 250 μg.

In some embodiments, the amount of the compound sufficient to improve vision, treat vision deterioration, treat an ocular disorder or condition, and/or decrease CST in the eye of the individual in need thereof is more than about 10 μg. In some embodiments, the amount of the compound sufficient to improve vision, treat vision deterioration, treat an ocular disorder or condition, and/or decrease CST in the eye of the individual in need thereof is about 10 μg to about 500 μg (e.g., about 50 μg to about 250 μg).

In some embodiments, the amount of the compound sufficient to improve vision, treat vision deterioration, treat an ocular disorder or condition, and/or decrease CST in the eye of the individual in need thereof is about 50 μg to about 250 μg. In some embodiments, the amount of the compound sufficient to improve vision, treat vision deterioration, treat an ocular disorder or condition, and/or decrease CST in the eye of the individual in need thereof is less than about 175 μg. In some embodiments, the amount of the compound sufficient to improve vision, treat vision deterioration, treat an ocular disorder or condition, and/or decrease CST in the eye of the individual in need thereof is less than about 156 μg (e.g., less than about 100 μg). In some embodiments, the amount of the compound sufficient to improve vision, treat vision deterioration, treat an ocular disorder or condition, and/or decrease CST in the eye of the individual in need thereof is about 75 μg to about 175 μg. In some embodiments, the amount of the compound sufficient to improve vision, treat vision deterioration, treat an ocular disorder or condition, and/or decrease CST in the eye of the individual in need thereof is about 50 μg to about 110 μg (e.g., about 78 μg to about 106 μg).

In some embodiments, one implant comprises about 70 μg of an active agent described herein (e.g., dexamethasone). In some embodiments, two implants comprises about 140 μg of an active agent described herein (e.g., dexamethasone). In some embodiments, the implant is intravitreally administered to the eye of the individual in need thereof in an amount of about 10 μg to about 500 μg of the compound. In some embodiments, the implant is intravitreally administered to the eye of the individual in need thereof in an amount of about 250 μg or less of the compound. In some embodiments, the implant is intravitreally administered to the eye of the individual in need thereof in an amount of about 100 μg or less of the compound.

In some embodiments, the implant is intravitreally administered to the eye of the individual in need thereof in an amount of less than about 175 μg of the compound. In some embodiments, the implant is intravitreally administered to the eye of the individual in need thereof in an amount of about 75 μg to about 175 μg of the compound. In some embodiments, the implant is intravitreally administered to the eye of the individual in need thereof in an amount of about 78 μg to about 106 μg of the compound.

In some embodiments, another implant is (e.g., intravitreally) administered to the eye of the individual in need thereof, such as before the one or more implant is completed degraded (e.g., about 35% of the one or more implant by mass remains).

In some embodiments, the composition or implant comprises at least 70% weight by weight (w/w) (e.g., 70% w/w or more, 80% w/w or more, 90% w/w or more, 95% w/w or more, or 99% w/w or more) of the compound. In some embodiments, the composition or implant comprises at least 95% w/w (e.g., 95% w/w or more, 96% w/w or more, 97% w/w or more, 98% w/w or more, or 99% w/w or more) of the compound.

In some embodiments, the implant is a fiber, a fiber mesh, a woven fabric, a non-woven fabric, a pellet, a cylinder (e.g., a cylinder, a rod, or a cylindrical-shaped implant), a hollow tube, a microparticle, a nanoparticle, or a shaped article. In some embodiments, the implant is a cylinder.

In some embodiments, the implant releases the compound or dexamethasone in its free form in an amount sufficient to improve vision, treat vision deterioration, treat an ocular disorder or condition, and/or decrease CST in the eye of the individual in need thereof.

In some embodiments, the implant releases the compound or dexamethasone in its free form over a period of at least a day. In some embodiments, the implant releases the compound or dexamethasone in its free form over a period of at least a week. In some embodiments, the implant releases the compound or dexamethasone in its free form over a period of at least a month. In some embodiments, the implant releases the compound or dexamethasone in its free form over a period of 6 months or more.

In some embodiments, the individual in need thereof has been diagnosed with a posterior ocular disease or disorder.

In some embodiments, the individual in need thereof has been diagnosed with a macular edema.

In some embodiments, the individual in need thereof has been diagnosed with a retinal disorder. In some embodiments, the retinal disorder is a retinal vein occlusion (RVO).

In some embodiments, the individual in need thereof has been diagnosed with diabetic macular edema (DME), macular edema secondary to retinal vein occlusion (RVO), or uveitis.

In some embodiments, the individual in need thereof has been diagnosed with DME or RVO. In some embodiments, the individual in need thereof has been diagnosed with DME. In some embodiments, the individual in need thereof has been diagnosed with RVO (e.g., central RVO).

In some embodiments, the individual in need thereof has a deterioration of vision. In some embodiments, the deterioration of vision is perceived by the individual and/or measured, such as, by a subjective method (e.g., vision test).

Provided in some embodiments herein is a kit comprising (i) an (e.g., intravitreal) injector, and (ii) no more than two and no less than one implant, the implant comprising a compound described herein in an amount of about 250 micrograms (μg) or less, wherein the implant comprises at least 90% w/w of the compound. In some embodiments, one implant is pre-loaded into the (e.g., intravitreal) injector. In some embodiments, two implants are pre-loaded into the (e.g., intravitreal) injector.

Provided herein is use of an implant described herein for manufacture of a medicament for use in a method described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A illustrates a reduction in central subfield thickness (CST) of macular edema in an individual with diabetic macular edema (DME) over a period of 12 months (12 Mo) in an individual who received one or more implant described herein, such as an implant comprising a compound having a structure described herein in an amount described herein.

FIG. 1B shows a significant decrease in CST over a period of 12 months (12 Mo) in an individual who received one or more implant described herein, such as one or more implant comprising a compound having a structure described herein in an amount described herein.

FIG. 1C shows a maintenance in vision acuity (e.g., best corrected visual acuity (BCVA)) over a period of 12 months (12 Mo) for an individual who received one or more implant described herein, such as an implant comprising a compound having a structure described herein in an amount described herein.

FIG. 1D illustrates a reduction in central subfield thickness (CST) of macular edema in a second individual with diabetic macular edema (DME) over a period of 12 months (12 Mo) in an individual who received one or more implant described herein, such as an implant comprising a compound having a structure described herein in an amount described herein.

FIG. 1E shows a decrease in CST over a period of 12 months (12 Mo) in the second individual who received one or more implant described herein, such as one or more implant comprising a compound having a structure described herein in an amount described herein.

FIG. 1F shows a maintained improvement in visual acuity (e.g., best corrected visual acuity (BCVA)) over a period of 12 months (12 Mo) for the second individual who received one or more implant described herein, such as an implant comprising a compound having a structure described herein in an amount described herein.

FIG. 2A illustrates a reduction in CST of macular edema in an individual with diabetic macular edema (DME) over a period of 12 months (12 Mo) in an individual who received two implants described herein, each implant comprising a compound having a structure described herein in an amount described herein.

FIG. 2B shows a substantial decrease in CST over a period of 12 months (12 Mo) in an individual who received two implants described herein, each implant comprising a compound having a structure described herein in an amount described herein.

FIG. 2C shows an improvement in visual acuity (e.g., BCVA) over a period of 12 months (12 Mo) for an individual who received two implants described herein, each implant comprising a compound having a structure described herein in an amount described herein.

FIG. 2D illustrates a reduction in CST of macular edema in a second individual with diabetic macular edema (DME) over a period of 8 months (8 Mo) in an individual who received two implants described herein, each implant comprising a compound having a structure described herein in an amount described herein.

FIG. 2E shows a decrease in CST over a period of 8 months (8 Mo) in a second individual who received two implants described herein, each implant comprising a compound having a structure described herein in an amount described herein.

FIG. 2F shows an improvement in visual acuity (e.g., BCVA) over a period of 8 months (8 Mo) for a second individual who received two implants described herein, each implant comprising a compound having a structure described herein in an amount described herein.

FIG. 3A illustrates a reduction in CST of macular edema in an individual with macular edema secondary to a retinal vein occlusion (RVO) over a period of 12 months (12 Mo) in an individual who received an implant described herein, such as an implant comprising a compound having a structure described herein in an amount described herein.

FIG. 3B shows a substantial decrease in CST over a period of 12 months (12 Mo) in an individual who received an implant described herein, such as an implant comprising a compound having a structure described herein in an amount described herein.

FIG. 3C shows a substantial improvement in visual acuity (e.g., BCVA) over a period of 12 months (12 Mo) for an individual who received an implant described herein, such as an implant comprising a compound having a structure described herein in an amount described herein.

FIG. 4A illustrates a reduction in CST of macular edema in an individual with macular edema secondary to a RVO over a period of 8 months (8 Mo) in an individual who received two implants described herein, each implant comprising a compound having a structure described herein in an amount described herein.

FIG. 4B shows a substantial decrease in CST over a period of 8 months (8 Mo) in an individual who received two implants described herein, each implant comprising a compound having a structure described herein in an amount described herein.

FIG. 4C shows a maintenance in vision acuity (e.g., BCVA) over a period of 8 months (8 Mo) for an individual who received two implants described herein, each implant comprising a compound having a structure described herein in an amount described herein.

FIG. 4D illustrates a reduction in CST of macular edema in a second individual with RVO over a period of 9 months (9 Mo) in an individual who received two implants described herein, each implant comprising a compound having a structure described herein in an amount described herein.

FIG. 4E shows a decrease in CST over a period of 10 months (10 Mo) in a second individual who received two implants described herein, each implant comprising a compound having a structure described herein in an amount described herein.

FIG. 4F shows an improvement in visual acuity (e.g., BCVA) over a period of 10 months (10 Mo) for a second individual who received two implants described herein, each implant comprising a compound having a structure described herein in an amount described herein.

FIG. 5A shows a substantial decrease of mean change in CST over a period of 12 months (12 Mo) in individuals who have received one (grey) or more (black) implant described herein, such as one or more implant comprising a compound having a structure described herein in an amount described herein.

FIG. 5B shows an improvement in visual acuity (e.g., as measured by mean change in BCVA) over a period of 12 months (12 Mo) in individuals who have received one (grey) or more (black) implant described herein, such as one or more implant comprising a compound having a structure described herein in an amount described herein.

FIG. 5C shows a comparison between the mean change in CST over a period of 12 months (12 Mo) in individuals who have received either a dexamethasone implant (Implant A) or one (grey) or more (black) implant described herein, such as one or more implant comprising a compound having a structure described herein in an amount described herein.

FIG. 5D shows a comparison between the mean change in visual acuity (e.g., as measured by mean change in BCVA) over a period of 12 months (12 Mo) in individuals who have received either a dexamethasone implant (Implant A) or one (grey) or more (black) implant described herein, such as one or more implant comprising a compound having a structure described herein in an amount described herein.

FIG. 6A shows a similar change in intraocular pressure (IOP) over a period of 12 months (12 Mo) in control eyes (grey) versus eyes of individuals who have received one or more implant described herein (black), such as one or more implant comprising a compound having a structure described herein in an amount described herein.

FIG. 6B shows a comparable change in IOP over a period of 12 months (12 Mo) in control eyes (grey) versus eyes of individuals who have received two implants described herein (black), each implant independently comprising a compound having a structure described herein in an amount described herein.

FIG. 6C shows change in IOP over a period of 12 months (12 Mo) in control eyes (grey) versus eyes of individuals who have RVO and receive one implant described herein (black), such as an implant comprising a compound having a structure described herein in an amount described herein.

FIG. 6D shows change in IOP over a period of 12 months (12 Mo) in control eyes (grey) versus eyes of individuals who have RVO and received two implants described herein (black), each implant independently comprising a compound having a structure described herein in an amount described herein.

FIG. 6E shows change in IOP over a period of 6 months (6 Mo) in control eyes (grey) versus eyes of individuals who have DME and received one implant described herein (black), such as an implant comprising a compound having a structure described herein in an amount described herein.

FIG. 6F shows change in IOP over a period of 6 months (6 Mo) in control eyes (grey) versus eyes of individuals who have DME and received two implants described herein (black), each implant independently comprising a compound having a structure described herein in an amount described herein.

FIG. 7A shows a decrease of mean change in CST over a period of 6 months (6 Mo) in individuals who have DME and received one (grey) or more (black) implant described herein, such as one or more implant comprising a compound having a structure described herein in an amount described herein.

FIG. 7B shows a maintenance in visual acuity (e.g., as measured by mean change in BCVA) over a period of 6 months (6 Mo) in individuals who have DME and received one (grey) or more (black) implant described herein, such as one or more implant comprising a compound having a structure described herein in an amount described herein.

FIG. 7C shows a comparison between the mean change in CST over a period of 6 months (6 Mo) in individuals who have DME and received either a dexamethasone implant (Implant A) or one (grey) or more (black) implant described herein, such as one or more implant comprising a compound having a structure described herein in an amount described herein. The curves for Implant A¹ and Implant A² represent data from two different clinical studies with Implant A.

FIG. 7D shows a comparison between the visual acuity (e.g., as measured by mean change in BCVA) over a period of 6 months (6 Mo) in individuals who have DME and received either a dexamethasone implant (Implant A) or one (grey) or more (black) implant described herein, such as one or more implant comprising a compound having a structure described herein in an amount described herein. The curves for Implant A¹ and Implant A² represent data from two different clinical studies with Implant A.

FIG. 7E shows a comparison between the mean change in CST over a period of 6 months (6 Mo) in individuals who have DME and either received anti-VEGF injections or two implants described herein, each implant comprising a compound having a structure described herein in an amount described herein.

FIG. 7F shows a comparison between the visual acuity (e.g., as measured by mean change in BCVA) over a period of 6 months (6 Mo) in individuals who have DME and either received anti-VEGF injections or two implants described herein, each implant comprising a compound having a structure described herein in an amount described herein.

FIG. 8 shows mean change in CST for individuals with RVO who have received anti-VEGF injections and two implants described herein (in series), each implant comprising a compound having a structure described herein in an amount described herein.

FIG. 9 shows mean change in CST for individuals with DME who have received anti-VEGF injections and an implant described herein (in series), such as an implant comprising a compound having a structure described herein in an amount described.

### DETAILED DESCRIPTION OF THE INVENTION

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

“Pharmaceutically acceptable salt” includes both acid and base addition salts. A pharmaceutically acceptable salt of any one of the pharmacological agents described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Amino” refers to the —NH₂ radical.

“Cyano” refers to the —CN radical.

“Nitro” refers to the —NO₂ radical.

“Oxo” refers to the ═O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl(optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Poly cyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g.—S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}. —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, itis intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

Unless stated specifically otherwise herein, each instance of radical indicates that a hydrogen (i.e., a hydrogen radical (H•)) is removed from a free form of a compound provided herein, such as any steroid described herein. In some instances, the radical is an oxygen radical (O•). In some instances, the removal of the hydrogen radical from the compound provided herein, such as any steroid described herein, provides a radical of a steroid that is taken together with any point of a linker provided herein (e.g., L or L^{A}) to form a bond (e.g., between the linker and the steroid radical). In some instances, the bond (e.g., between the linker and the steroid radical) is a C—O bond.

Unless stated specifically otherwise herein, each instance of implant herein refers to a solid or semi-solid (e.g., amorphous) composition suitable for implantation into an individual, such as an eye of an individual described herein. Unless stated specifically otherwise herein, a compound described herein can be formed into an implant, an article, or a coating (e.g., on the surface of a device), each of which being suitable for implantation into an individual, such as into an eye of an individual described herein.

Provided herein in some embodiments herein are compounds that can be formed into implants that are useful for treating inflammation or an inflammatory condition, such as being described herein, in an individual in need thereof, such as an individual described herein. In some embodiments, the implants are useful for treating an ocular disorder or condition, such as being described herein, in an individual in need thereof, such as an individual described herein. In some instances, the ocular disorder or condition is associated with inflammation. In some embodiments, the implants are useful for improving or maintaining vision (e.g., vision acuity) in an individual in need thereof, such as an individual described herein. In some embodiments, the implants are useful for treating vision deterioration in an individual in need thereof, such as an individual described herein. In some embodiments, the implants are useful for decreasing central subfield thickness (CST) in an individual in need thereof, such as an individual described herein.

Provided in some embodiments herein are compounds comprising a first radical (D1) and a second radical (D2)(e.g., having the formula: D1-L-D2). In certain instances, D1 is a first steroid radical, L is a linker, and D2 is a second steroid radical. In some embodiments, Lis a hydrolyzable linker, such that when the compound (or an implant comprising a compound) of formula D1-L-D2 is (e.g., ophthalmically) administered (or when present in or otherwise exposed to an aqueous environment, such as a buffering solution, tears, serum, or the like), D1 and D2 are released (e.g., in their free, non-radical form). In certain instances, the (e.g., covalent) joining of a group D1 to a group of D2 through a linker L (e.g., D1-L-D2) provides a compound that releases an effective amount of D1 and D2 in their free form for a (e.g., prolonged) period of time.

Provided in certain instances herein is a platform for providing compounds and implants (e.g., with high drug content, low excipient content (e.g., that would otherwise need to be removed), and other benefits, such as described herein) that provide long-lasting release of therapeutics (e.g., steroids) in biological and therapeutic applications, such as in ocular (e.g., implant) administration.

In some instances, compounds provided herein are processable into forms (e.g., implants, coatings, or other bodies), such as that are capable of being administered to (e.g., an eye of) an individual in need thereof. In some instances, such compounds are processable without the need for additional excipients or materials (e.g., controlled release polymers, matrices, or other components). In certain instances, no or low amounts of additional excipients or materials facilitate high levels of drug delivery, while limiting impact of drug delivery (e.g., a small implant can have high quantities of drug).

In certain embodiments, a composition (e.g., an implant) described herein comprises any compound described herein, such as a compound having the structure of anyone of the compounds described herein, or a compound described in any of U.S. Pat. Nos. 10,632,075, 10,588,862, 10,959,954, 10,945,958, U.S. Patent Publication Number 2021/0113457, U.S. Patent Publication Number 2021/0030667, U.S. Patent Publication Number 2021/0205222, U.S. Patent Publication Number 2022/0089635, and U.S. patent application Ser. No. 17/625,708, each of which is incorporated herein in their entirety. In certain embodiments, a compound described herein comprises any steroid or linker described in any of U.S. Pat. Nos. 10,632,075, 10,588,862, 10,959,954, 10,945,958, U.S. Patent Publication Number 2021/0113457, U.S. Patent Publication Number 2021/0030667, U. S. Patent Publication Number 2021/0205222, U.S. Patent Publication Number 2022/0089635, and U.S. patent application Ser. No. 17/625,708, each of which is incorporated herein in their entirety.

In some embodiments, an implant described herein is a solid. In some embodiments, an implant described herein is amorphous.

In some embodiments, an implant described herein is a fiber, a fiber mesh, a woven fabric, a non-woven fabric, a pellet, a cylinder, a hollow tube, a microparticle, a nanoparticle, or a shaped article.

In some embodiments, an implant described herein is a cylindrical-shaped implant. In some embodiments, an implant described herein is a cylinder or a rod. In some instances, the implant has cut edges, slight bend, or the like.

In some embodiments, the composition (e.g., implant, coating, or the like) is described in any of U.S. Pat. Nos. 10,632,075, 10,588,862, 10,959,954, 10,945,958, U.S. Patent Publication Number 2021/0113457, U.S. Patent Publication Number 2021/0030667, U.S. Patent Publication Number 2021/0205222, U.S. Patent Publication Number 2022/0089635, and U.S. patent application Ser. No. 17/625,708, each of which is incorporated herein in their entirety.

In certain instances, such compounds (or implants comprising such compounds) are administered to (e.g., implanted into) an individual, such that sustained and/or otherwise controlled (e.g., local) delivery of the drug is achieved. In some instances, delivery of the compounds (e.g., in the form of an implant, coating, etc.) facilitate delivery of a drug component for an extended period of time, such as for weeks, months, or more. In certain instances, compounds, formulations, and implants provided herein facilitate the long-term delivery of drugs to an individual in need thereof, such as without the need for frequent dosing. For example, as discussed herein, steroids are often formulated and administered as eye drops, such as with daily administration. In some instances, rigid compliance to frequent administration is required to maintain (e.g., optimal) therapeutic efficacy. With the compounds provided herein, however, long term delivery of such drugs can be achieved from weeks, months, or more, with infrequent administration (e.g., once a year, twice a year, or the like).

Provided in some embodiments herein are compounds, such as described herein, (e.g., pharmaceutical) compositions comprising compounds described herein, and methods of making and using compounds provided herein. In some embodiments, methods of using the compounds provided herein include methods of treating disorders in individuals in need thereof, such as disorders treatable by D1 and/or D2 (e.g., in their free form). In some embodiments, methods of treatment provided herein comprise methods of treating inflammatory and/or ocular disorders, such as post ocular disorders, such as macular edema (e.g., diabetic macular edema (DME)) and/or retinal disorders (e.g., retinal disorders causing macular edema, such as retinal vein occlusion (RVO)). It is to be understood that disclosures of methods provided herein explicitly include disclosures of pharmaceutical compositions comprising (e.g., an effective amount) of a compound provided herein for such uses.

In certain embodiments, a compound described herein, such as a compound having the structure of any one of the compounds described herein, is formed into an article, implant, coating (e.g., on a device), or other body using any method described in any of U.S. Pat. Nos. 10,632,075, 10,588,862, 10,959,954, 10,945,958, U.S. Patent Publication Number 2021/0113457, U.S. Patent Publication Number 2021/0030667, U.S. Patent Publication Number 2021/0205222, U.S. Patent Publication Number 2022/0089635, and U.S. patent application Ser. No. 17/625,708, each of which is incorporated herein in their entirety. In some instances, the article, implant, coating (e.g., on a device), or other body is formed by heat molding or solvent processing a composition comprising any compound described herein, such as a compound having the structure of any one of the compounds described herein.

In some instances, provided herein is a compound that delivers a therapeutically effective amount of (e.g., a free form of) a steroid, such as a steroid described herein (e.g., dexamethasone).

In some embodiments, a compound provided herein has a structure represented by Formula (I):

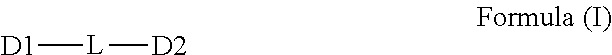

or a pharmaceutically acceptable salt or solvate thereof,
wherein,
   D1 and D2 are each independently a steroid radical (e.g., a dexamethasone radical); and
   L is a (e.g., hydrolyzable) linker.

In some embodiments, D1 and D2 are each independently selected from the group consisting of an angiostatic steroid (e.g., anecortave), a benign steroid (e.g., cholesterol), a corticosteroid (e.g., glucocorticoid or mineralcorticoid, such as dexamethasone), a sex steroid, a neurosteroid, an aminosteroid, or a secosteroid.

In some embodiments, D1 is a dexamethasone radical.

In some embodiments, D2 is a dexamethasone radical.

In some embodiments, D1 and D2 are each independently a dexamethasone radical.

In some embodiments, D1 is C21-dexamethasone (radical) (e.g., the carbon atom (e.g., C21) indicating which carbon atom the oxygen radical (e.g., of dexamethasone) is attached to the linker described herein)).

In some embodiments, D2 is C21-dexamethasone (radical) (e.g., the carbon atom (e.g., C21) indicating which carbon atom the oxygen radical (e.g., of dexamethasone) is attached to the linker described herein)).

In some embodiments, D1 and D2 are each C21-dexamethasone (radical).

In some embodiments, the linker described herein (e.g., L) is a hydrolyzable linker.

In some embodiments, L comprises one or more linker group, each linker group being independently selected from the group consisting of a bond, substituted or unsubstituted alkyl, substituted or unsubstituted heteroalkyl, substituted or unsubstituted alkoxy, substituted or unsubstituted carbocyclyl, substituted or unsubstituted aryl, or substituted or unsubstituted heteroaryl. In some embodiments, L comprises one or more linker group, each linker group being independently selected from the group consisting of substituted or unsubstituted alkyl, substituted or unsubstituted heteroalkyl, substituted or unsubstituted alkoxy, substituted or unsubstituted carbocyclyl, substituted or unsubstituted aryl, or substituted or unsubstituted heteroaryl. In some embodiments, L is substituted or unsubstituted alkyl, substituted or unsubstituted heteroalkyl, substituted or unsubstituted alkoxy, or substituted or unsubstituted aryl. In some embodiments, L is a bond. In some embodiments, L is substituted or unsubstituted alkyl. In some embodiments, L is substituted or unsubstituted heteroalkyl. In some embodiments, Lis substituted or unsubstituted alkoxy. In some embodiments, Lis substituted or unsubstituted aryl. In some embodiments, Lis substituted or unsubstituted heteroaryl.

In some embodiments, a linker described herein is a bond, alkyl, heteroalkyl, or alkoxy, wherein the alkyl, heteroalkyl, or alkoxy is optionally substituted. In some embodiments, the alkyl, heteroalkyl, or alkoxy are each independently substituted with one or more substituent, each substituent being independently selected from the group consisting of oxo, hydroxyl, alkoxy, thiol, thioalkoxy, silicone, amino, alkyl, heteroalkyl, cycloalkyl, heterocycloalkyl, aryl or heteroaryl, wherein the alkyl, heteroalkyl, cycloalkyl, heterocycloalkyl, aryl or heteroaryl are optionally further substituted (e.g., with halogen or hydroxyl). In some embodiments, the linker is alkyl (alkylene) and the alkyl (alkylene) is substituted with one or more groups selected from —OH, halo, oxo, alkyl, heteroalkyl, cycloalkyl, and heterocycloalkyl.

In some embodiments, the linker is heteroalkyl.

In some embodiments, the linker comprises at least one oxo. In some embodiments, the linker comprises two oxo groups. In some embodiments, the linker comprises one or more ester, carbonate, anhydride, carbamate, ester, or any combination thereof. In some embodiments, the linker comprises at least one carbamate. In some embodiments, the linker comprises at least one carbonate. In some embodiments, the linker comprises at least one ester. In some embodiments, the linker comprises two or more esters.

In some embodiments, the linker comprises one or more linker groups, each linker group being independently selected from the group consisting of —O—, —S—, optionally substituted alkylene (e.g., alkenyl, alkynyl, branched (e.g., polypropylene), haloalkyl), optionally substituted heteroalkylene (e.g., polyTHF), and optionally substituted cycloalkylene. In some embodiments, the linker comprises one or more linker groups, each linker group being independently selected from the group consisting of alkyl, alkoxy, and cycloalkyl, wherein the alkyl, alkoxy, or cycloalkyl are optionally substituted.

In some embodiments, the linker comprises one or more linker groups selected from —O—, —S—, unsubstituted alkylene, (CH₂CH₂)ₙ, (CHCH)ₙ, O(CH₂CH₂O)ₙ, (CH₂CH₂O)ₙ, and (CH(CH₃)C(═O)O)ₙ, wherein n is 1-20. In some embodiments, the linker is unsubstituted alkylene, (CH₂CH₂)ₙ, (CHCH)ₙ, O(CH₂CH₂O)ₙ, (CH₂CH₂O)ₙ, (CH(CH₃)C(═O)O)ₙ, or (CH₂CH₂)ₙC═O(CH(CH₃)C(═O)O)ₙ, wherein n is 1-20.

In some embodiments, the linker is alkyl (alkylene) substituted with one or more groups selected from —OH, halo, oxo, alkyl, heteroalkyl, cycloalkyl, and heterocycloalkyl. In some embodiments, the linker is unsubstituted alkyl (alkylene). In some embodiments, the linker is heteroalkyl (heteroalkylene) substituted with one or more groups selected from halo or alkyl. In some embodiments, the linker is unsubstituted heteroalkyl (heteroalkylene). In some embodiments, the linker is selected from the group consisting of: —(CR₂)_{y}—, —O(CR₂)_{y}O—, —O(CR₂)_{y}—, —(CR₂)_{y}O—, and —O(CR₂CR₂O)_{y}—, wherein y is 1-10 and each R is independently selected from the group consisting of H, halogen, alkyl, or is taken together with another R to form an optionally substituted cycloalkyl. In some embodiments, each R is independently selected from H, alkyl, or is taken together with another R to form an optionally substituted cycloalkyl. In some embodiments, the one or more R is taken together with one or more other R to form a bridged cycloalkyl (e.g., a bridged cycloalkylene).

In some embodiments, the linker is hydrolyzed in a buffered solution. In some embodiments, the linker is hydrolytically labile. In some embodiments, the linker is hydrolyzed by water. In some embodiments, the linker is hydrolyzed by an enzyme. In some embodiments, the enzyme is a hydrolase (e.g., a protease or an esterase). In some embodiments, the enzyme is an esterase.

In some instances, longer linkers (e.g., at least 3-4 (e.g., carbon) atoms between an —O—) are preferred because, e.g., in some instances, shorter linkers result in increased melting point, increased T_{g}, increased crystallinity, decreased processability, or any combination thereof. In some instances, compounds comprising linkers having 7 or more carbon atoms (e.g., between an —O—) are not processable (e.g., because the compounds lack enough rigidity to form a sufficient crystal lattice).

In some embodiments, —(CH₂)ₓ-cycloalkyl-(CH₂)_{y}—, wherein x and y are each independently 0-3. In some embodiments, x and y are each independently 0-2. In some embodiments, x and y are each independently 0 or 1. In some embodiments, x and y are each 0.

In some embodiments, x and y are each 1.

In some embodiments, the linker is hydrolyzed in a buffered solution. In some embodiments, the linker is hydrolyzed by an enzyme. In some embodiments, the enzyme is a hydrolase (e.g., a protease or an esterase).

In some embodiments, the linker is a bond.

In some embodiments, a compound provided herein has a structure represented by Formula (I-A):

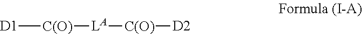

or a pharmaceutically acceptable salt or solvate thereof,
wherein,
   D1 and D2 are each independently a steroid radical (e.g., a dexamethasone radical); and
   L^{A} is substituted or unsubstituted alkyl or substituted or unsubstituted heteroalkyl.

In some embodiments, D1 and D2 are described hereinabove.

In some embodiments, D1 and D2 are each represented by the same steroid radical structure.

In some embodiments, D1 is a radical of dexamethasone, prednisolone, triamcinolone, hydrocortisone, loteprednol, or fluocinolone.

In some embodiments, D2 is a radical of dexamethasone, prednisolone, triamcinolone, hydrocortisone, loteprednol, or fluocinolone.

In some embodiments, D1 and D2 are each a dexamethasone radical. In some embodiments, D1 and D2 are each independently connected to the linker (e.g., L or L^{A}) by an oxygen radical, thereby forming a C—O bond (e.g., with the linker).

In some embodiments, the linker (e.g., L or L^{A}) is unsubstituted alkyl. In some embodiments, L^{A} is methylene, ethylene, propylene, butylene, pentylene, or hexylene.

In some embodiments, the linker (e.g., L or L^{A}) is unsubstituted heteroalkyl. In some embodiments, the linker (e.g., L or L^{A}) is comprises one or more ethyleneglycol unit (e.g., —O(CH₂CH₂O)—). In some embodiments, the linker (e.g., L or L^{A}) is comprises one to six ethyleneglycol units.

In some embodiments, the linker (e.g., L or L^{A}) is —O(CH₂CH₂O)ₙ, where n is an integer of 1-10. In some embodiments, n is an integer of 3-5. In some embodiments. n is 3.

In some embodiments, the linker (e.g., L or L^{A}) is triethylene glycol (TEG).

In some embodiments, either or both of D1 or D2 are attached to the linker (e.g., L or L^{A}) through an oxygen radical of the D1 or D2 (e.g., thereby forming a C—O bond). In some embodiments, an oxygen radical of the D1 and an oxygen radical of the D2 are each independently attached to a C(O). In some embodiments, the C(O) of Formula (I-A) is attached to D1 and D2, respectively.

In some embodiments, the linker (e.g., L or L^{A}) is attached to any hydroxyl group of any steroid described herein, such as a hydroxyl (e.g., at the C11-, C17-, C21-position), a carboxylate, a phosphate, or an enolizable ketone (e.g., at the C1-position) of any steroid described herein.

In some embodiments, the linker (e.g., L or L^{A}) is attached to a hydroxyl group at the C21-position of any steroid described herein.

In some embodiments, the linker (e.g., L or L^{A}) is attached to a hydroxyl group at the C21-position of dexamethasone.

In some embodiments, either or both of D1 or D2 are attached to L or L^{A} through an oxygen radical of D1 or D2.

In some embodiments, D1 and D2 are attached to L or L^{A} through an oxygen radical of D1 or D2.

In some embodiments, D1 and D2 are attached to L through an oxygen radical of D1 or D2.

In some embodiments, D1 and D2 are attached to L through an oxygen radical of D1 and D2.

In some embodiments, D1 and D2 are attached to L^{A} through an oxygen radical of D1 or D2.

In some embodiments, D1 and D2 are attached to L^{A} through an oxygen radical of D1 and D2.

In some embodiments, a compound described herein is Compound 1, which is represented by the structure:

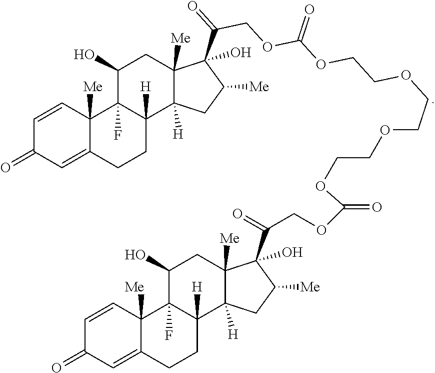

Provided in some embodiments herein is a composition comprising a compound described herein, or a pharmaceutically-acceptable salt thereof. In some embodiments, the composition comprises the compound in an amount of about 250 micrograms (μg) or less. In some embodiments, the composition comprises at least 90% w/w of the compound.

Provided in some embodiments herein is a pharmaceutical implant or article comprising a compound described herein, or a pharmaceutically-acceptable salt thereof. In some embodiments, the pharmaceutical implant or article comprises the compound in an amount of about 250 micrograms (μg) or less. In some embodiments, the pharmaceutical implant or article comprises at least 90% w/w of the compound.

Provided in some embodiments herein is an implant comprising a compound described herein, or a pharmaceutically-acceptable salt thereof. In some embodiments, the implant comprises the compound in an amount of about 250 micrograms (μg) or less. In some embodiments, the implant comprises at least 90% w/w of the compound.

Provided in some embodiments herein is a dosage form comprising a compound described herein, or a pharmaceutically-acceptable salt thereof. In some embodiments, the dosage form comprises the compound in an amount of about 250 micrograms (μg) or less. In some embodiments, the dosage form comprises at least 90% w/w of the compound.

In some instances, the composition, dosage form, or pharmaceutical implant or article comprises at most about 500 micrograms (μg) of the compound described herein (e.g., Compound 1). In some instances, the composition, dosage form, or pharmaceutical implant or article comprises no more than about 500 μg of the compound described herein (e.g., Compound 1). In some instances, the composition, dosage form, or pharmaceutical implant or article comprises 500 μg or less, 400 μg or less, 300 μg or less, 200 μg or less, 100 μg or less, or 50 μg or less of the compound described herein (e.g., Compound 1). In some instances, the composition, dosage form, or pharmaceutical implant or article comprises 250 μg or less, 150 μg or less, or 50 μg or less of the compound described herein (e.g., Compound 1). In some instances, the composition, dosage form, or pharmaceutical implant or article comprises about 250 μg or less of the compound described herein (e.g., Compound 1). In some instances, the composition, dosage form, or pharmaceutical implant or article comprises no more than about 175 μg of the compound described herein (e.g., Compound 1). In some instances, the pharmaceutical implant or article comprises about 156 μg or less of the compound described herein (e.g., Compound 1). In some instances, the composition, dosage form, or pharmaceutical implant or article comprises about 150 μg or less of the compound described herein (e.g., Compound 1).

In some instances, the composition, dosage form, or pharmaceutical implant or article comprises at least about 10 micrograms (μg) of the compound described herein (e.g., Compound 1). In some instances, the pharmaceutical implant or article comprises 10 μg or more, 100 μg or more, 200 μg or more, 300 μg or more, 400 μg or more, or 500 μg or more of the compound described herein (e.g., Compound 1). In some instances, the composition, dosage form, or pharmaceutical implant or article comprises 10 μg or more, 50 μg or more, or 100 μg or more of the compound described herein (e.g., Compound 1). In some instances, the composition, dosage form, or pharmaceutical implant or article comprises about 50 μg or more of the compound described herein (e.g., Compound 1). In some instances, the composition, dosage form, or pharmaceutical implant or article comprises about 75 μg or more of the compound described herein (e.g., Compound 1). In some instances, the composition, dosage form, or pharmaceutical implant or article comprises about 100 μg or more of the compound described herein (e.g., Compound 1).

In some instances, the composition, dosage form, or pharmaceutical implant or article comprises about 10 μg to about 500 μg of the compound described herein (e.g., Compound 1). In some instances, the composition, dosage form, or pharmaceutical implant or article comprises about 50 μg to about 250 μg of the compound described herein (e.g., Compound 1). In some instances, the composition, dosage form, or pharmaceutical implant or article comprises about 50 μg to about 150 μg of the compound described herein (e.g., Compound 1). In some instances, the composition, dosage form, or pharmaceutical implant or article comprises about 50 μg to about 110 μg of the compound described herein (e.g., Compound 1). In some instances, the composition, dosage form, or pharmaceutical implant or article comprises about 100 μg to about 150 μg of the compound described herein (e.g., Compound 1).

In some instances, one implant described herein, such as a Compound 1 IVT implant, comprises about 70 μg of an active agent described herein (e.g., dexamethasone). In some instances, two implants described herein, such as a Compound 1 IVT implant, comprises about 140 μg of an active agent described herein (e.g., dexamethasone).

In some instances, the composition, dosage form, or pharmaceutical implant or article comprises about 75 μg to about 175 μg of the compound described herein (e.g., Compound 1). In some instances, the composition, dosage form, or pharmaceutical implant or article comprises about 78 to about 116 μg of the compound described herein (e.g., Compound 1). In some instances, the composition, dosage form, or pharmaceutical implant or article comprises about 78 to about 106 μg of the compound described herein (e.g., Compound 1). In some instances, the composition, dosage form, or pharmaceutical implant or article comprises about 75 μg to about 175 μg of Compound 1. In some instances, the pharmaceutical implant or article comprises about 75 to about 110 μg of the compound described herein (e.g., Compound 1). In some instances, the pharmaceutical implant or article comprises about 75 to about 110 μg of Compound 1. In some instances, the composition, dosage form, or pharmaceutical implant or article comprises about 78 to about 106 μg of Compound 1. In some instances, the pharmaceutical implant or article comprises about 92 μg of Compound 1.

In some instances, the composition, dosage form, or pharmaceutical implant or article comprises at most about 500 micrograms (μg) of dexamethasone by free (e.g., free form) weight. In some instances, the composition, dosage form, or pharmaceutical implant or article comprises 500 μg or less, 400 μg or less, 300 μg or less, 200 μg or less, 100 μg or less, or 50 μg or less of dexamethasone by free (e.g., free form) weight. In some instances, the composition, dosage form, or pharmaceutical implant or article comprises 200 μg or less, 100 μg or less, or 50 μg or less of dexamethasone by free (e.g., free form) weight. In some instances, the composition, dosage form, or pharmaceutical implant or article comprises about 200 μg or less of dexamethasone by free (e.g., free form) weight. In some instances, the composition, dosage form, or pharmaceutical implant or article comprises about 100 μg or less of dexamethasone by free (e.g., free form) weight.

In some instances, the composition, dosage form, or pharmaceutical implant or article comprises at least about 10 micrograms (μg) of dexamethasone by free (e.g., free form) weight. In some instances, the composition, dosage form, or pharmaceutical implant or article comprises 10 μg or more, 100 μg or more, 200 μg or more, 300 μg or more, 400 μg or more, or 500 μg or more of dexamethasone by free (e.g., free form) weight. In some instances, the composition, dosage form, or pharmaceutical implant or article comprises 10 μg or more, 50 μg or more, or 100 μg or more of dexamethasone by free (e.g., free form) weight. In some instances, the composition, dosage form, or pharmaceutical implant or article comprises about 50 μg or more of dexamethasone by free (e.g., free form) weight.

In some instances, the composition, dosage form, or pharmaceutical implant or article comprises about 10 μg to about 500 μg of dexamethasone by free (e.g., free form) weight. In some instances, the composition, dosage form, or pharmaceutical implant or article comprises about 50 μg to about 200 μg of dexamethasone by free (e.g., free form) weight. In some instances, the composition, dosage form, or pharmaceutical implant or article comprises about 50 μg to about 100 μg of dexamethasone by free (e.g., free form) weight.

In some instances, the composition, dosage form, or pharmaceutical implant or article comprises about 70 μg of dexamethasone by free (e.g., free form) weight.

In some embodiments, a composition, a dosage form, or an implant or article described herein comprises at least 50 wt. % (at least 60 wt. %, at least 70 wt. %, at least 80 wt. %, at least 90 wt. %, at least 95 wt. %, at least 98 wt. %, or the like) of a compound described herein or pharmaceutically acceptable salt thereof.

In some embodiments, a composition, a dosage form, or an implant or article described herein comprises at least 70 wt. % (e.g., 70 wt. % or more, 80 wt. % or more, 90 wt. % or more, 95 wt. % or more, 98 wt. % or more, or 99 wt. % or more) of a compound described herein or pharmaceutically acceptable salt thereof.

In some embodiments, a composition, a dosage form, or an implant or article described herein comprises at least 90 wt. % (e.g., about 90 wt. % or more, about 95 wt. % or more, or about 99 wt. % or more) of a compound described herein or pharmaceutically acceptable salt thereof.

In some embodiments, a composition, a dosage form, or an implant or article described herein comprises at least 95% w/w (e.g., 95% w/w or more, 96% w/w or more, 97% w/w or more, 98% w/w or more, or 99% w/w or more) of a compound described herein or pharmaceutically acceptable salt thereof.

In some embodiments, a composition, a dosage form, or an implant or article described herein undergoes surface erosion to release the compound and/or a steroid radical (in its free form) described herein. In some embodiments, a steroid radical described herein is released (in its free form) from a pharmaceutical implant or article described herein at near zero-order in a buffered solution or in vivo. In some embodiments, a steroid radical described herein is released (in its free form) from a pharmaceutical implant or article described herein at 37° C. in 100% bovine serum or at 37° C. in phosphate buffered saline (PBS) at a rate such that t₁₀ is greater than or equal to 1/10 of t₅₀. In some embodiments, a steroid radical described herein are released (in its free form) from a pharmaceutical implantor article described herein at 37° C. in 1% fetal bovine serum (FBS) in phosphate buffered saline (PBS) at a rate such that t₁₀ is greater than or equal to 1/10 of t₅₀.

In some embodiments, a composition, a dosage form, or an implant described herein releases a compound described herein or dexamethasone in its free form over a period of at least a day. In some embodiments, an implant described herein releases a compound described herein or dexamethasone in its free form over a period of one day or more, five days or more or more, ten days or more, fifteen days or more, twenty days or more, or twenty-five days or more.

In some embodiments, a composition, a dosage form, or an implant described herein releases a compound described herein or dexamethasone in its free form over a period of at least a week. In some embodiments, an implant described herein releases a compound described herein or dexamethasone in its free form over a period of one week or more, two weeks or more, or three weeks or more.

In some embodiments, a composition, a dosage form, or an implant described herein releases a compound described herein or dexamethasone in its free form over a period of at least a month. In some embodiments, an implant described herein releases a compound described herein or dexamethasone in its free form over a period of one month or more, two months or more, three months or more, four months or more, five months or more, six months or more, seven months or more, or eight months or more.

In some embodiments, a composition, a dosage form, or an implant described herein releases a compound described herein or dexamethasone in its free form over a period of about six months.

In some embodiments, a composition, a dosage form, or an implant described herein releases a compound described herein or dexamethasone in its free form over a period of at least a year.

Provided in some embodiments herein is a pharmaceutical composition comprising any compound provided herein, or a pharmaceutically-acceptable salt thereof, and at least one pharmaceutically-acceptable excipient.

In some embodiments, an implant, article, or composition described herein is in a form suitable for ophthalmic administration. In some embodiments, the ophthalmic administration is intraocular, intracameral, intravitreal, suprachoroidal, punctal, retrobulbar, or sub conjunctival.

In some embodiments, an implant, article, or composition described herein is in a form suitable for intravitreal administration.

In some embodiments, an article or implant described herein is at least partially biodegradable. In some embodiments, an article or implant described herein is non-biodegradable. In some embodiments, removal of an article or implant described herein is not required (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)). In some embodiments, an article or implant described herein is not removed from an individual receiving the article or implant (e.g., because the implant is completely or almost completely (e.g., bio- or physiologically) degraded or degradable (e.g., at least 80 wt. %, at least 85 wt. %, at least 90 wt. %, at least 95 wt. %, at least 98 wt. %, at least 99 wt. %, or the like)).

In some embodiments, provided herein is an intravitreal implant comprising Compound 1. In some embodiments, the intravitreal implant comprises Compound 1 in an amount of at least 95% (w/w). In some embodiments, the intravitreal implant comprises Compound 1 in an amount of at least 98% (w/w). In some embodiments, the intravitreal implant consists essentially of Compound 1. In some embodiments, the intravitreal implant consists of Compound 1.

Provided herein is use of an implant described herein, such as an implant described hereinabove, for manufacture of a medicament for use in any method described herein, such as a method described herein below.

Provided herein is a method of treating an inflammatory disease or disorder in an individual in need thereof, the method comprising administering to the individual any compound, pharmaceutically acceptable salt, implant, article, or composition described herein.

In some embodiments, the individual in need thereof has been diagnosed with an inflammatory disease or disorder.

Provided herein is a method of treating an ophthalmic disease or disorder in an individual in need thereof, the method comprising administering to the individual any compound, pharmaceutically acceptable salt, implant, article, or composition described herein.

In some embodiments, the condition, disease, or disorder is intraocular melanoma; acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema (e.g., cystoid macular edema (CME), diabetic macular edema (DME), and macular edema from retinal vein occlusion); multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, retinoblastoma, and glaucoma.

In some embodiments, the condition, disease, or disorder is selected from the group consisting of ocular inflammation, diabetic macular edema (DME), posterior inflammation, anterior inflammation, macular degeneration (e.g., wet age-related macular degeneration (wAMD) or dry AMD), post-cataract surgery, retinal vein occlusion (RVO), or uveitis.

In some embodiments, the individual in need thereof has been diagnosed with an ocular disease or disorder.

In some embodiments, the individual in need thereof has been diagnosed with a posterior ocular disease or disorder.

In some embodiments, the individual in need thereof has been diagnosed with a macular edema.

In some embodiments, the individual in need thereof has been diagnosed with a retinal disorder. In some embodiments, the retinal disorder is a RVO.

In some embodiments, the individual in need thereof has been diagnosed with DME, macular edema secondary to RVO, or uveitis.

In some embodiments, the individual has been diagnosed with DME or RVO.

In some embodiments, the individual has been diagnosed with DME.

In some embodiments, the individual has been diagnosed with RVO. In some embodiments, the individual has been diagnosed with central RVO.

In some embodiments, the individual in need thereof has been diagnosed with uveitis. In some embodiments, the individual in need thereof has been diagnosed with non-infectious uveitis (NIU). In some embodiments, the individual in need thereof has been diagnosed with chronic NIU.

Provided herein is a method of treating an ocular disorder or condition in an individual in need thereof, the method comprising administering to the individual any compound, pharmaceutically acceptable salt, implant, article, or composition described herein.

Provided herein is a method of treating a posterior ocular disorder or condition in an individual in need thereof, the method comprising administering to the individual any compound, pharmaceutically acceptable salt, implant, article, or composition described herein.

In some embodiments, the disorder or condition is a macular edema.

In some embodiments, the disorder or condition is DME.

In some embodiments, the disorder or condition is a retinal disorder.

In some embodiments, the disorder or condition is a RVO. In some embodiments, the RVO is a central RVO.

In some embodiments, the disorder or condition is DME, macular edema secondary to RVO, or uveitis.

In some embodiments, the disorder or condition is DME or RVO.

In some embodiments, the disorder or condition is DME.

In some embodiments, the disorder or condition is macular edema secondary to RVO.

In some embodiments, the disorder or condition is uveitis.

In some embodiments, the disorder or condition is NIU.

Provided herein is a method of improving and/or maintaining vision (e.g., vision acuity) in an individual in need thereof, the method comprising administering to the individual any compound, pharmaceutically acceptable salt, implant, article, or composition described herein.

Provided herein is a method for improving and/or maintaining vision (e.g., visual acuity) in an individual in need thereof. In some embodiments, the method comprises administering to an eye of the individual in need thereof one or more (e.g., one or two) implant(s) described herein.

In some embodiments, the method comprises intravitreally administering to an eye of the individual in need thereof one or more (e.g., one or two) implant(s) described herein. In some embodiments, each implant comprising a compound having a structure represented by Formula (I): D1-L-D2. In some embodiments, D1 and D2 are each independently a steroid radical. In some embodiments, D1 and D2 are each independently a dexamethasone radical. In some embodiments, L is a linker. In some embodiments, L is a hydrolyzable linker. In some embodiments, D1, D2, and L are described elsewhere herein. In some embodiments, the one or more implant described herein collectively comprises an amount of the compound sufficient to improve and/or maintain vision in the eye of the individual in need thereof.

In some embodiments, as described herein, an individual (or a population of individuals) administered one or more implant described herein, such as Compound 1 IVT implant, has an improvement in visual acuity (e.g., as measured by one or more ETDRS letter test), such as compared to a baseline measurement. In some embodiments, as described herein, an individual (or a population of individuals) administered one or more implant described herein, such as Compound 1 IVT implant, has a decrease in CST, such as compared to a baseline measurement. In some embodiments, as described herein, an individual (or a population of individuals) administered one or more implant described herein, such as Compound 1 IVT implant, has an improvement in visual acuity (e.g., as measured by one or more ETDRS letter test), such as compared to a baseline visual acuity measurement, and a decrease in CST, such as compared to a baseline CST measurement. In some instances, an improvement in vision and a decrease in CST are measures of the effectiveness of an implant described herein, such as for treating an ocular disease or disorder (in the individual) described herein (e.g., DME or RVO).

In some embodiments, as described herein, an individual (or a population of individuals) administered one or more implant described herein, such as Compound 1 IVT implant, has a visual acuity (e.g., as measured by one or more ETDRS letter test) that remains the same, such as compared to levels prior to being administered the one or more one or more implant described herein, such as Compound 1 IVT implant. In some embodiments, as described herein, an individual (or a population of individuals) administered one or more implant described herein, such as Compound 1 IVT implant, has a decrease in CST, such as compared to a baseline measurement. In some embodiments, as described herein, an individual (or a population of individuals) administered one or more implant described herein, such as Compound 1 IVT implant, has a visual acuity (e.g., as measured by one or more ETDRS letter test) that remains the same, such as compared to a baseline visual acuity measurement, and a decrease in CST, such as compared to a baseline CST measurement. In some instances, a maintenance in vision and a decrease in CST are measures of the effectiveness of an implant described herein, such as for treating an ocular disease or disorder (in the individual) described herein (e.g., DME or RVO).

In some embodiments, one or more implant described herein, such as Compound 1 IVT implant, is administered to one or more eye of each individual of a population of individuals in need thereof (e.g., described herein). In some embodiments, one or more implant described herein, such as a Compound 1 IVT implant, is intravitreally administered to one or more eye of each individual of a population of individuals in need thereof. In some embodiments, one or more Compound 1 IVT implant is intravitreally administered to one or more eye of each individual of a population of individuals in need thereof.

In some embodiments, an individual of a population of individuals (e.g., described herein) is an individual described herein, such an individual in need thereof (e.g., an individual with DME or RVO).

In some embodiments, the population of individuals (e.g., described herein) have RVO.

In some embodiments, the population of individuals (e.g., described herein) have DME.

In some embodiments, one implant described herein is administered to one or more eye of each individual of the population of individuals (e.g., described herein).

In some embodiments, two implants described herein are administered to one or more eye of each individual of the population of individuals (e.g., described herein). In some embodiments, the two implants are simultaneously administered (into the eye).

In some embodiments, the vision (e.g., the visual acuity) of the individual improves within about two months. In some embodiments, the vision (e.g., the visual acuity) of the individual improves within about two months, within about one month, within about two weeks, or within about one week. In some embodiments, the vision (e.g., the visual acuity) of the individual improves within about two months of intravitreally administering the implant to the eye of the individual in need thereof.

In some embodiments, vision (e.g., visual acuity and/or vision improvement) is maintained for about one month or more. In some embodiments, vision (e.g., visual acuity and/or vision improvement) is maintained for about one month or more, about two months or more, about three months or more, about four months or more, about five months or more, about six months or more, about seven months or more, about eight months or more, about nine months or more, about ten months or more, about eleven months or more, or about twelve months or more. In some embodiments, vision (e.g., visual acuity and/or improvement) is maintained for about one month or more after intravitreally administering the implant to the eye of the individual in need thereof. In some embodiments, vision (e.g., visual acuity and/or improvement) is maintained indefinitely. In some embodiments, vision (e.g., visual acuity and/or improvement) is maintained indefinitely after intravitreally administering the implant to the eye of the individual in need thereof.

In some embodiments, an improvement in vision is measured by an initial (e.g., first) visual acuity measurement and a subsequent (e.g., second) visual acuity measurement. In some embodiments, the subsequent (e.g., second) visual acuity measurement increases compared to the initial (e.g., first) visual acuity measurement.

In some embodiments, the initial (e.g., first) visual acuity measurement is a baseline visual acuity measurement.

In some embodiments, the second visual acuity measurement is subsequent to the first visual acuity measurement. In some embodiments, the second visual acuity measurement is obtained within about nine months of the first visual acuity measurement. In some embodiments, the second visual acuity measurement is obtained within about 12 months of the first visual acuity measurement. In some embodiments, the second visual acuity measurement is obtained within about 9 months, within about 8 months, within about 7 months, within about 6 months, within about 5 months, within about 4 months, within about 3 months, within about 2 months, or within about 1 month of the first visual acuity measurement. In some embodiments, the second visual acuity measurement is obtained within about 12 months, within about 11 months, within about 10 months, within about 9 months, within about 8 months, within about 7 months, within about 6 months, within about 5 months, within about 4 months, within about 3 months, within about 2 months, or within about 1 month of the first visual acuity measurement.

In some instances, a successful response to the implant described herein provides an improvement in vision acuity for at least one month. In some instances, a successful response to the implant described herein provides an improvement in vision acuity for one month or more, two months or more, three months or more, four months or more, five months or more, six months or more, seven months or more, or eight months or more. In some instances, a successful response to the implant described herein provides an improvement in vision acuity for at least four months.

In some embodiments, the first and second visual acuity are measured by (using) an ETDRS chart, a Snellen chart, or a logMAR chart. In some embodiments, the first and second visual acuity are measured by (using) an ETDRS chart. In some embodiments, the first and second visual acuity are measured by (using) a Best Corrected Visual Acuity (BCVA) method.

In some embodiments, the first and second visual acuity are measured by BCVA. In some instances, a BCVA measurement indicates the vision assessment and/or vision acuity of an individual. In some instances, an individual is wearing corrective lens (e.g., glasses or contacts) during the BCVA.

In some instances, one or more implant described herein is administered to the individual and the individual has no substantial change in vision between a first (e.g., baseline) visual acuity measurement (e.g., from an ETDRS letter test) and a second (e.g., subsequent) visual acuity measurement (e.g., from an ETDRS letter test). In some instances, one or more implant described herein is administered to the individual and the individual has a decrease in vision between a first (e.g., baseline) visual acuity measurement (e.g., from an ETDRS letter test) and a second (e.g., subsequent) visual acuity measurement (e.g., from an ETDRS letter test). In some instances, one or more implant described herein is administered to the individual and the individual has a substantial improvement in vision between a first (e.g., baseline) visual acuity measurement (e.g., from an ETDRS letter test) and a subsequent visual acuity measurement. In some instances, the subsequent visual acuity measurement is at least a second visual acuity measurement, such as a third visual acuity measurement, a fourth visual acuity measurement, a fifth visual acuity measurement, or more. In some instances, the subsequent visual acuity measurement is a second visual acuity measurement, a third visual acuity measurement, a fourth visual acuity measurement, or a fifth visual acuity measurement. In some instances, the subsequent visual acuity measurement is measured one or more week after the one or more implant is administered to the individual. In some instances, the subsequent visual acuity measurement is measured one or more month, two or more months, three or more months, four or more months, five or more months, six or more months, seven or more months, eight or more months, nine or more months, ten or more months, eleven or more months, or twelve or more months, after the one or more implant is administered to the individual. In some instances, one or more implant described herein is administered to the individual and the individual has an overall improvement in vision (e.g., visual acuity) during a course of treatment. In some instances, one or more implant described herein is administered to the individual and the individual has an overall improvement in vision (e.g., visual acuity) over a period of about one month or more, three months or more, six months or more, nine months or more, or twelve months or more. In some instances, one or more implant described herein is administered to the individual and the vision (e.g., visual acuity) of the individual remains substantially the same during a course of treatment. In some instances, one or more implant described herein is administered to the individual and the vision (e.g., visual acuity) of the individual remains substantially the same while the compound is released from the implant in the eye of the individual.

In some embodiments, the vision of the individual is improved by at least 5 letters (by BCVA). In some embodiments, the vision of the individual is improved by 5 letters or more, 10 letters or more, 15 letters or more, 20 letters or more, 25 letters or more, or 30 or more letters (by BCVA). In some embodiments, the vision of the individual is improved by 5 or more letters, 6 or more letters, 7 or more letters, 8 or more letters, 9 or more letters, 10 or more letters, or 11 or more letters (by BCVA). In some embodiments, the vision of the individual is improved for any period of time described herein, such as for at least one month or more, two months or more, three months or more, four months or more, five months or more, six months or more, seven months or more, eight months or more, nine months or more, ten months or more, eleven months or more, or twelve months or more. In some embodiments, the vision of the individual is measured by one or more ETDRS letter test.

In some embodiments, the vision of the individual remains improved for at least about 2 months. In some embodiments, the vision of the individual remains improved for at least about 2 months after intravitreally administering the implant to the eye of the individual in need thereof.

In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more after intravitreally administering the implant to the eye of the individual in need thereof.

In some embodiments, a second visual acuity measurement remains higher than a first (e.g., baseline) visual acuity measurement. In some embodiments, a second visual acuity measurement remains higher than a first (e.g., baseline) visual acuity measurement after intravitreally administering the implant to the eye of the individual in need thereof. In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more after intravitreally administering the implant to the eye of the individual in need thereof.

In some embodiments, the improvement in vision of the individual (or the population of individuals) does not substantially decrease during a course of treatment. In some embodiments, the improvement in vision of the individual (or the population of individuals) does not substantially decrease during the first two months or more, three months or more, four months or more, five months or more, or six months or more of treatment. In some embodiments, the improvement in vision of the individual (or the population of individuals) does not substantially decrease over a period of at least two months or more, three months or more, four months or more, five months or more, or six months or more. In some embodiments, the individual (or the population of individuals) is administered one or two implants described herein and has RVO. In some embodiments, the improvement in vision of the individual (or the population of individuals) does not decrease relative to a prior measurement (e.g., a third measurement remains higher than a second measurement) within the first three months of the implant being administered. In some embodiments, the individual (or the population of individuals) is administered two implants described herein and has DME. In some embodiments, the improvement in vision of the individual (or the population of individuals) does not decrease relative to a prior measurement (e.g., a third measurement remains higher than a second measurement) within the first five months of the implant being administered.

In some embodiments, the improvement in vision of the individual (or the population of individuals) continues to increase during a course of treatment. In some embodiments, the improvement in vision of the individual (or the population of individuals) continues to increase during the first 6 months or more, 7 months or more, 8 months or more, 9 months or more, 10 months or more, 11 months or more, or 12 months or more of treatment. In some embodiments, the improvement in vision of the individual (or the population of individuals) continues to increase over a period of about 6 months or more, 7 months or more, 8 months or more, 9 months or more, 10 months or more, 11 months or more, or 12 months or more. In some embodiments, the individual (or the population of individuals) is administered one or two implants described herein and has RVO. In some embodiments, the individual (or the population of individuals) is administered two implants described herein and has DME.

In some embodiments, an individual in need thereof described herein has a deterioration of vision. In some embodiments, the deterioration of vision is perceived by the individual. In some embodiments, the deterioration of vision is measured. In some embodiments, the deterioration of vision is perceived by the individual and measured. In some embodiments, the deterioration of vision is perceived by the individual or measured. In some embodiments, the deterioration of vision is measured by a subjective method. In some embodiments, the deterioration of vision is measured by a vision test, such as a vision test described elsewhere herein.

Provided herein is a method for treating vision deterioration in an individual in need thereof, the method comprising administering to the individual any compound, pharmaceutically acceptable salt, implant, article, or composition described herein.

Provided herein is a method for treating vision deterioration in an individual in need thereof. In some embodiments, the method comprises administering to an eye of the individual in need thereof one or more (e.g., one or two) implant(s) described herein. In some embodiments, the method comprises intravitreally administering to an eye of the individual in need thereof one or more (e.g., one or two) implant(s) described herein. In some embodiments, each implant comprising a compound having a structure represented by Formula (I): D1-L-D2. In some embodiments, D1 and D2 are each independently a steroid radical. In some embodiments, D1 and D2 are each independently a dexamethasone radical. In some embodiments, L is a linker. In some embodiments, L is a hydrolyzable linker. In some embodiments, D1, D2, and L are described elsewhere herein. In some embodiments, the one or more implant described herein collectively comprises an amount of the compound sufficient to treat the vision deterioration in the eye of the individual in need thereof.

In some embodiments, treating vision deterioration comprises reducing a rate of vision deterioration, maintaining a (current) level of vision, and/or improving vision in the individual in need thereof. In some embodiments, treating vision deterioration comprises reducing a rate of vision deterioration. In some embodiments, treating vision deterioration comprises maintaining a (current) level of vision. In some embodiments, treating vision deterioration comprises improving vision in the individual in need thereof.

In some embodiments, treating vision deterioration comprises reducing a rate of vision deterioration, such as by at least 5% (e.g., 5%, 10%, 20%, 30% or more reduction in the rate of vision deterioration). In some embodiments, treating vision deterioration comprises reducing a rate of vision deterioration, such as by 40% or less (e.g., 40%, 30%, 20%, 10% or less reduction in the rate of vision deterioration).

In some embodiments, treating vision deterioration comprises maintaining a level of vision. In some embodiments, treating vision deterioration comprises maintaining a current level of vision. In some embodiments, treating vision deterioration comprises maintaining a baseline level of vision. In some embodiments, treating vision deterioration comprises maintaining an improved level of vision.

In some embodiments, treating vision deterioration comprises improving vision in the individual in need thereof, such as by at least 5% (e.g., 5%, 10%, 20%, 300% or more improvement in the vision of the individual in need thereof). In some embodiments, improving vision in the individual in need thereof, such as by 40% or less (e.g., 40%, 30%, 20%, 10% or less improvement in the vision of the individual in need thereof).

In some embodiments, the vision deterioration of the individual is maintained or improves within about two months. In some embodiments, the vision deterioration of the individual is maintained or improves within about two months, within about one month, within about two weeks, or within about one week. In some embodiments, the vision deterioration of the individual is maintained or improves within about two months of intravitreally administering the implant to the eye of the individual in need thereof.

In some embodiments, maintenance or improvement in vision is measured by a first (e.g., baseline) visual acuity measurement (e.g., from an ETDRS letter test) and a second (e.g., sub sequent) visual acuity measurement (e.g., from an ETDRS letter test) (e.g., wherein the second visual acuity measurement is maintained or increases compared to the first (e.g., baseline) visual acuity measurement). In some embodiments, the first and second visual acuity measurements are described elsewhere herein, such as hereinabove.

In some embodiments, the vision of the individual is maintained or improved by at least 5 letters (by BCVA). In some embodiments, the vision of the individual is maintained or improved by 5 letters or more, 10 letters or more, 15 letters or more, 20 letters or more, or 25 letters or more (by BCVA). In some embodiments, the vision of the individual is maintained or improved for any period of time described herein, such as for at least one month or more, two months or more, three months or more, four months or more, five months or more, six months or more, seven months or more, eight months or more, nine months or more, ten months or more, eleven months or more, or twelve months or more. In some embodiments, the vision of the individual is measured by one or more ETDRS letter test.

In some embodiments, the vision (e.g., as measured by one or more ETDRS letter test) of each individual of a population of individuals described herein is measured at a timepoint before (e.g., a baseline measurement) and at various timepoints after an implant described herein is administered to an individual of the population of individuals. In some embodiments, a change in vision (improvement) in an individual is determined by measuring the difference between a baseline vision measurement and a vision (improvement) measurement measured after an implant described herein is administered to the individual. In some embodiments, the vision (improvement) measurement is measured at least one week after an implant described herein is administered to the individual. In some embodiments, the vision (improvement) measurement is measured at month one (M1), month two (M2), month three (M3), month four (M4), month five (M5), month six (M6), month seven (M7), month eight (M8), month nine (M9), month ten (M10), month eleven (M11), or month twelve (M12) of treatment. In some embodiments, a mean change in vision (improvement) of the population of individuals is determined by averaging each change in vision (improvement) of each individual at the respective timepoint. For example, a mean change in vision (improvement) at month 1 can be determined by averaging each change in vision (improvement) of each individual at M1, and so on until a mean change in vision (improvement) at each month of treatment is determined.

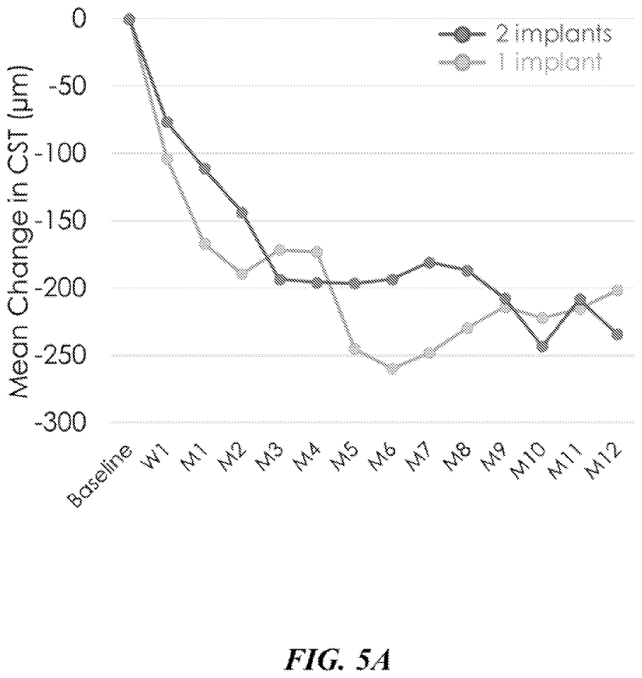

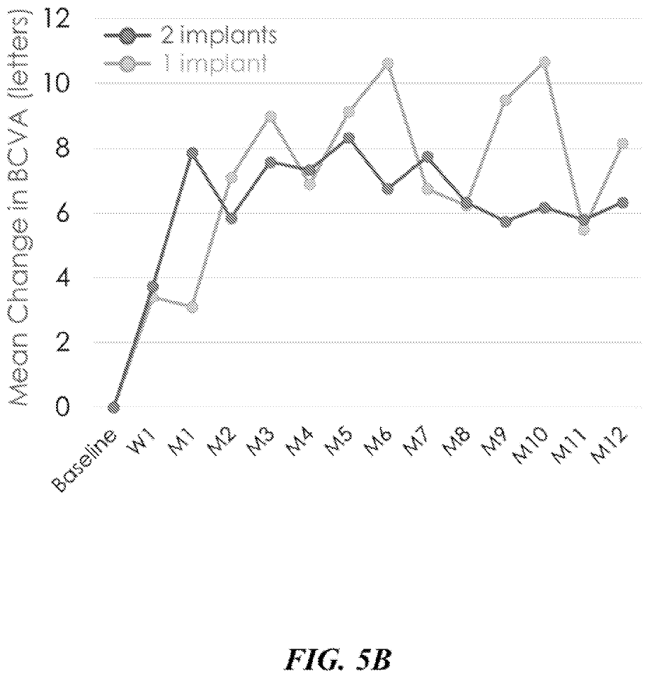

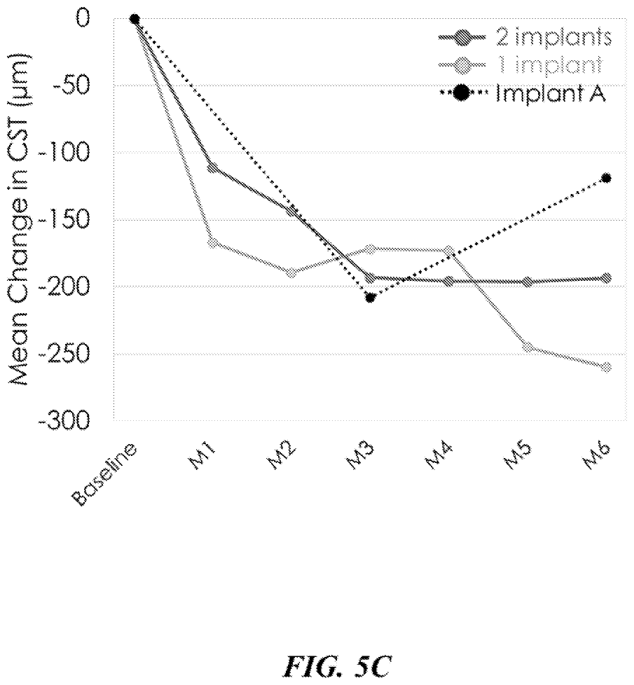

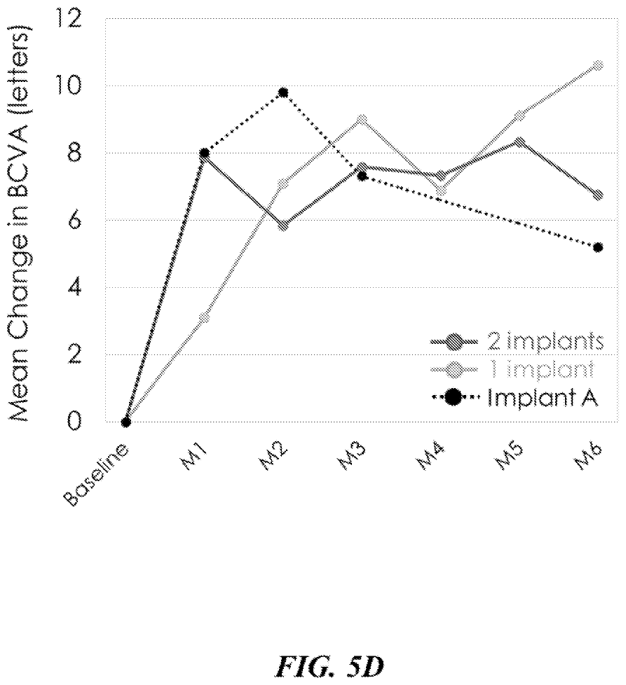

In some instances, a mean change, such as a mean change in best corrected visual acuity (BCVA), central subfield thickness (CST), or intraocular pressure (IOP), refers to change in a measurement at a specific timepoint (treatment week one (W1), treatment month 1 (M1), etc.) for an entire population of individuals, such a population of individuals described herein. For example, the mean change in BCVA from baseline to treatment week 1 (W1) is about 4 letters when one or two implants are injected into an eye of a population of individuals with retinal vein occlusion (RVO) (see FIG. 5B)

In some embodiments, the population of individuals have a mean change in vision (improvement) that increases. In some embodiments, the population of individuals have a mean change in vision (improvement) that increases over a period of at least about one week. In some embodiments, the population of individuals have a mean change in vision (improvement) that increases over a period of about 1 month or more, ab out 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, the population of individuals have a second mean change in vision (improvement) measurement that increases compared to a first mean change in vision (improvement) measurement. In some embodiments, the first mean change in vision (improvement) measurement for the population is compared to a vision (improvement) measurement at first timepoint (e.g., baseline, at one-month of treatment, at two-months of treatment, etc.). In some embodiments, the first mean change in vision (improvement) measurement for the population is at a first timepoint (e.g., at one-month of treatment, at two-months of treatment, etc.). In some embodiments, the second mean change in vision (improvement) measurement for the population is at a second timepoint (at one-month of treatment, at two-months of treatment, etc.). In some embodiments, the first timepoint is before the second timepoint.

In some embodiments, the vision of the population of individuals (e.g., as measured by one or more ETDRS letter test) improves by at least 5 letters, as measured by BCVA. In some embodiments, the vision of the population of individuals (e.g., as measured by one or more ETDRS letter test) improves by 5 or more letter, 6 or more letters, 7 or more letters, 8 or more letters, 9 or more letters, as measured by BCVA.

In some embodiments, the vision deterioration remains treated for at least about 2 months. In some embodiments, the vision deterioration remains treated for at least about 2 months after intravitreally administering the implant to the eye of the individual in need thereof. In some embodiments, a second visual acuity measurement (e.g., slightly) lower than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, a second visual acuity measurement remains (e.g., slightly) lower than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more after intravitreally administering the implant to the eye of the individual in need thereof.

In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more after intravitreally administering the implant to the eye of the individual in need thereof.

In some embodiments, the vision deterioration of the individual is improved for at least about 2 months. In some embodiments, the vision deterioration of the individual is improved for at least about 2 months after intravitreally administering the implant to the eye of the individual in need thereof. In some embodiments, a second visual acuity measurement (e.g., slightly) lower than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, a second visual acuity measurement remains (e.g., slightly) lower than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more after intravitreally administering the implant to the eye of the individual in need thereof. In some embodiments, a second visual acuity measurement remains higher than a first (e.g., baseline) visual acuity measurement for about 2 months or more, about 3 months or more, about 4 months or more, about 5 months or more, about 6 months or more, about 7 months or more, about 8 months or more, about 9 months or more, about 10 months or more, about 11 months or more, or about 12 months or more after intravitreally administering the implant to the eye of the individual in need thereof.

Provided in some embodiments herein is a method for treating an ocular disorder or condition in an individual in need thereof. In some embodiments, the method comprises (e.g., intravitreally) administering to an eye of the individual in need thereof a first (e.g., initial) implant and a second (e.g., subsequent) implant. In some embodiments, the second implant is administered to the eye of the individual in need thereof after a certain period of time described herein and/or after a reversion of treatment (e.g., as measured by CST or visual acuity). In some embodiments, the second implant is administered to the eye of the individual in need thereof no less than one month after the first implant is administered to the eye of the individual. In some embodiments, the second implant is administered to the eye of the individual in need thereof one month or more, two months or more, three months or more, four months or more, five months or more, or six months or more after the first implant is administered to the eye of the individual. In some embodiments, the second implant is administered to the eye of the individual in need thereof one month or more, two months or more, three months or more, four months or more, five months or more, or six months or more, seven months or more, eight months or more, nine months or more, ten months or more, eleven months or more, or twelve months or more after the first implant is administered to the eye of the individual.

In some embodiments, each implant comprises a compound having a structure represented by Formula (I): D1-L-D2. In some embodiments, D1 and D2 are each independently a steroid radical. In some embodiments, D1 and D2 are each independently a dexamethasone radical. In some embodiments, L is a linker. In some embodiments, L is a hydrolyzable linker. In some embodiments, D1, D2, and L are described elsewhere herein. In some embodiments, the one or more implant described herein collectively comprises an amount of the compound sufficient to treat the ocular disorder or condition in the eye of the individual in need thereof.

In some embodiments, a third implant is administered to the eye of the individual in need thereof.

In some embodiments, the third implant is administered to the eye of the individual in need thereof about six months or more after the first implant is administered to the eye of the individual in need thereof.

In some embodiments, the third implant is administered to the eye of the individual in need thereof about three months or more after the second implant is administered to the eye of the individual in need thereof.

In some embodiments, the third implant is administered to the eye of the individual in need thereof about six months or more after the second implant is administered to the eye of the individual in need thereof.

In some embodiments, the second implant is administered to the eye of the individual within at least about a month of the first implant. In some embodiments, the second implant is administered to the eye of the individual one month or more, about two months or more, about three months or more, about four months or more, about five months or more, about six months or more, about seven months or more, about eight months or more, about nine months or more, about ten months or more, about eleven months or more, or about twelve months or more of the first implant. In some embodiments, the second implant is administered to the eye of the individual within at least about three months of the first implant.

In some embodiments, the second implant is administered to the eye of the individual in need thereof at least six months after the first implant is administered to the eye of the individual in need thereof.

In some embodiments, the second implant is administered to the eye of the individual in need thereof no more than six months after the first implant is administered to the eye of the individual in need thereof. In some embodiments, the individual is lacking a response to the first implant, such as, lacking a decrease in CST of 5% or more compared to baseline, such as at or before month 3.

In some embodiments, the individual receives a second implant and a third implant within six months after the first implant is administered to the eye of the individual in need thereof.

In some embodiments, the third implant is administered subsequent to the first implant. In some embodiments, the third implant is administered subsequent to the second implant. In some embodiments, the third implant is administered subsequent to the first implant and the second implant.

In some embodiments, a subsequent implant (second implant, third implant, etc.) is (e.g., intravitreally) administered to the eye of the individual in need thereof before the one or more implant is completed degraded. In some embodiments, the (second implant, third implant, etc.) is (e.g., intravitreally) administered to the eye of the individual in need thereof when about 50% or less (by mass) of the one or more implant (e.g., the first implant, the second implant, or the like) remains in the eye of the individual. In some embodiments, the (second implant, third implant, etc.) is (e.g., intravitreally) administered to the eye of the individual in need thereof when about 40% or less (by mass) of the one or more implant (e.g., the first implant, the second implant, or the like) remains in the eye of the individual. In some embodiments, the (second implant, third implant, etc.) is (e.g., intravitreally) administered to the eye of the individual in need thereof when about 35% or less (by mass) of the one or more implant (e.g., the first implant, the second implant, or the like) remains in the eye of the individual. In some embodiments, the (second implant, third implant, etc.) is (e.g., intravitreally) administered to the eye of the individual in need thereof when about 0% to 50% (by mass) of the one or more implant (e.g., the first implant, the second implant, or the like) remains in the eye of the individual. In some embodiments, the (second implant, third implant, etc.) is (e.g., intravitreally) administered to the eye of the individual in need thereof when about 0% to 35% (by mass) of the one or more implant (e.g., the first implant, the second implant, or the like) remains in the eye of the individual. In some embodiments, the (second implant, third implant, etc.) is (e.g., intravitreally) administered to the eye of the individual in need thereof when about 35% (by mass) of the one or more implant (e.g., the first implant, the second implant, or the like) remains in the eye of the individual.

In some embodiments, a subsequent implant (second implant, third implant, etc.) is (e.g., intravitreally) administered to the eye of the individual in need thereof after the one or more implant (e.g., the first implant, the second implant, or the like) almost completely degrades (e.g., less than 5% (by mass) of the one or more implant remains) in the eye of the individual.

In some embodiments, a subsequent implant (second implant, third implant, etc.) is (e.g., intravitreally) administered to the eye of the individual in need thereof after the one or more implant (e.g., the first implant, the second implant, or the like) completely degrades in the eye of the individual.

In some embodiments, a subsequent implant (second implant, third implant, etc.) is (e.g., intravitreally) administered to the eye of the individual in need thereof after a period of time described herein and/or after a reversion of treatment (e.g., as measured by CST or visual acuity), such as of at least about 25% or more or 50% or more. In some embodiments, a subsequent implant (second implant, third implant, etc.) is (e.g., intravitreally) administered to the eye of the individual in need thereof after CST (measurement) of the individual (substantially) increases, such as an increase of about 25% or more or 50% or more from a prior CST (measurement). In some embodiments, a subsequent implant (second implant, third implant, etc.) is (e.g., intravitreally) administered to the eye of the individual in need thereof after visual acuity (measurement) of the individual (substantially) decreases, such as a decrease of about 25% or more or 50% or more from a prior visual acuity (measurement).

In some instances, an individual (or a population of individuals) described herein receive an angiogenesis inhibitor, such as a compound that prevents proliferation and/or formation of blood vessels (e.g., an anti-VEGF compound described herein, such as Anti-VEGF A or Anti-VEGF B). In some instances, an individual (or a population of individuals) described herein receive a steroid (e.g., a dexamethasone IVT implant described herein, such as Implant A).

In some instances, an individual (or a population of individuals) described herein receive a vascular endothelial growth factor (VEGF) treatment. In some instances, an individual (or a population of individuals) described herein receive an anti-VEGF treatment. In some instances, an individual (or a population of individuals) described herein receive an anti-VEGF injection described herein, before an implant described herein is administered to the individual (or the population of individuals). In some instances, the anti-VEGF injection comprises a VEGF inhibitor. In some instances, the anti-VEGF injection comprises an anti-vascular endothelial growth factor-A (anti-VEGF-A). In certain instances, one or more implants administered to the eye of the individual in need thereof after the anti-VEGF injection comprise a compound described herein. In certain instances, the anti-VEGF injection comprising an anti-VEGF-A compound is administered once per month to the eye of the individual in need thereof. In certain instances, a second implant, such as comprising a compound described herein (e.g., Compound 1), is administered to the eye of the individual in need thereof substantially less frequently than the anti-VEGF injection. In certain instances, an implant comprising a compound described herein (e.g., Compound 1) is administered to the eye of the individual in need thereof once every three months or less, once every 4 months or less, once every 5 months or less, or once every 6 months or less. In certain instances, the one or more implants comprising a compound described herein are administered to the eye of the individual in need thereof once every 6 months or less. In some embodiments, the individual is administered a second implant after the VEGF injection is administered to the individual. In some embodiments, the individual is administered a second implant one or more month after the anti-VEGF injection or a first implant is administered to the individual. In some embodiments, the individual is administered a second implant one or more year after the anti-VEGF injection or the first implant is administered to the individual. In some embodiments, the individual is administered the anti-VEGF injection and one or more implant concurrently. In some embodiments, the individual is administered two implants concurrently. In some embodiments, the individual is only administered one implant (e.g., every 6 months or more).

Provided in some embodiments herein is a method for treating an ocular disorder or condition in an individual in need thereof. In some embodiments, the method comprises administering to an eye of the individual in need thereof a first agent and one or more implant comprising a compound described herein, such as a compound having a structure represented by Formula (I). In some embodiments, the method comprises intravitreally administering to an eye of the individual in need thereof a pharmaceutical composition comprising a first agent (e.g., a steroid and/or an angiogenesis inhibitor, such as an anti-growth factor and/or an anti-angiogenic factor) and one or more implant comprising a compound described herein, such as a compound having a structure represented by Formula (I). In some embodiments, the method comprises intravitreally administering to an eye of the individual in need thereof an injection comprising a first agent (e.g., a steroid and/or an angiogenesis inhibitor, such as an anti-growth factor and/or an anti-angiogenic factor) and one or more implant comprising a compound described herein, such as a compound having a structure represented by Formula (I). In some embodiments, the method comprises intravitreally administering to an eye of the individual in need thereof a first agent and one or more implant comprising a compound described herein, such as a compound having a structure represented by Formula (I).

In some embodiments, the first agent is used to treat and/or improve the symptoms of macular edema. In some embodiments, the first agent is used to treat and/or improve the symptoms of diabetic macular edema (DME). In some embodiments, the first agent is used to treat and/or improve the symptoms of retinal vein occlusion (RVO).

In some embodiments, a method described herein further comprises injecting an injection comprising the first agent into the eye of the individual in need thereof. In some embodiments, a method described herein further comprises injecting an intravitreal (IVT) injection comprising the first agent into the eye of the individual in need thereof.

In some embodiments, the first agent and the one or more implant are administered to the eye of the individual sequentially. In some embodiments, the one or more implant is administered to the eye of the individual after the first agent is administered to the eye of the individual. In some embodiments, the one or more implant is administered to the eye of the individual at least one week after (e.g., after a washout period of) the first agent is administered to the eye of the individual. In some embodiments, the one or more implant is administered to the eye of the individual after a washout period of the first agent, such as after a period of about one week, one month, two months, or longer. In some instances, the washout period is a length of time that it takes for the first agent to no longer be efficacious and/or the ocular disease described herein is no longer being treated (e.g., signs and symptoms of the disease have retuned).

In some embodiments, the first agent is formulated as an injection. In some embodiments, the first agent is formulated as an intravitreal injection. In some embodiments, the first agent is formulated as an intravitreal anti-inflammatory agent injection or an intravitreal angiogenesis inhibitor injection. In some embodiments, the first agent is formulated as an intravitreal anti-inflammatory agent injection. In some embodiments, the first agent is formulated as an intravitreal angiogenesis inhibitor injection. In some embodiments, the first agent is formulated as an intravitreal steroid injection or an intravitreal anti-VEGF injection. In some embodiments, the first agent is formulated as an intravitreal steroid injection, such as an intravitreal dexamethasone implant described herein (e.g., Implant A). In some embodiments, the first agent is formulated as an intravitreal anti-VEGF injection (e.g., Anti-VEGF A or Anti-VEGF B). In some embodiments, the first agent is formulated as an eye drop.

In some embodiments, the first agent is an anti-inflammatory agent and/or an angiogenesis inhibitor.

In some embodiments, the first agent is an angiogenesis inhibitor. In some embodiments, the first agent is a compound that prevents proliferation and/or formation of blood vessels, such as an anti-VEGF compound described herein, such as Anti-VEGF A or Anti-VEGF B. In some embodiments, the first agent is an anti-growth factor and/or an anti-angiogenic factor. In some embodiments, the first agent is an anti-growth factor. In some embodiments, the first agent is an anti-angiogenic factor. In some embodiments, the first agent is an anti-growth factor and an anti-angiogenic factor. In some embodiments, the first agent is a vascular endothelial growth factor (VEGF) treatment. In some embodiments, the first agent is an anti-vascular endothelial growth factor (anti-VEGF) treatment. In some embodiments, the first agent is an anti-VEGF injection. In some embodiments, the first agent is a bispecific monoclonal antibody comprising vascular endothelial growth factor-A (VEGF-A) and angiopoietin-2 (Ang-2). In some embodiments, the anti-VEGF injection is ranibizumab, aflibercept, faricimab, or bevacizumab. In some embodiments, the anti-VEGF injection is ranibizumab or aflibercept. In some embodiments, the first agent is an VEGF antagonist. In some embodiments, the first agent is an VEGF inhibitor. In some embodiments, the first agent is an anti-vascular endothelial growth factor-A (anti-VEGF-A). In some embodiments, the first agent is ranibizumab or aflibercept. In some embodiments, the first agent is ranibizumab. In some embodiments, the first agent is aflibercept. In some embodiments, the first agent is faricimab. In some embodiments, the first agent is bevacizumab.

In some embodiments, the first agent is an anti-inflammatory agent. In some embodiments, the first agent is a steroid. In some embodiments, the first agent is dexamethasone. In some embodiments, the first agent is formulated as a steroid implant. In some embodiments, the first agent is a formulated as asteroid intravitreal (IVT) implant, such as a dexamethasone IVT implant described herein (e.g., Implant A). In some embodiments, the steroid implant described herein (e.g., Implant A) is administered to the eye of the individual.

In some embodiments, the one or more implant is administered after the first agent to extend treatment in the eye of the individual. In some embodiments, the one or more implant is administered after the first agent to extend treatment in the eye of the individual for about one month or more. In some embodiments, the one or more implant is administered after the first agent to extend treatment in the eye of the individual for about 3 months or more. In some embodiments, the one or more implant is administered after the first agent to extend treatment in the eye of the individual for about 6 months or more. In some embodiments, the one or more implant is administered after the first agent to extend treatment in the eye of the individual for about 9 months or more. In some embodiments, the one or more implant is administered after the first agent to extend treatment in the eye of the individual for about 12 months or more. In some embodiments, the one or more implant is administered after the first agent to maintain or extend a reduction of symptoms, improvement in visual acuity (e.g., as measured by BCVA), and/or reduction in CST in the one or more eye of the individual. In some embodiments, the one or more implant is administered after the first agent to maintain or extend a reduction of symptoms in the one or more eye of the individual. In some embodiments, the one or more implant is administered after the first agent to maintain or extend an improvement in visual acuity (e.g., as measured by BCVA) in the one or more eye of the individual. In some embodiments, the one or more implant is administered after the first agent to maintain or extend a reduction in CST in the one or more eye of the individual.

Provided in some embodiments herein is a method for treating an ocular disorder or condition in an individual in need thereof, the method comprising (e.g., intravitreally) administering to an eye of the individual in need thereof one or more implant no more frequently than once every three months. In some embodiments, each implant comprising a compound having a structure represented by Formula (I): D1-L-D2. In some embodiments, D1 and D2 are each independently a steroid radical. In some embodiments, D1 and D2 are each independently a dexamethasone radical. In some embodiments, L is a linker. In some embodiments, L is a hydrolyzable linker. In some embodiments, D1, D2, and L are described elsewhere herein. In some embodiments, the one or more implant described herein collectively comprises an amount of the compound sufficient to treat the ocular disorder or condition in the eye of the individual in need thereof.

In some embodiments, an implant described herein is administered to the individual in need thereof once every month or less, once every two months or less, three months or less, once every 4 months or less, once every 5 months or less, or once every 6 months or less. In some embodiments, an implant described herein is administered to the individual in need thereof once every three months. In some embodiments, an implant described herein is administered to the individual in need thereof once every six months.

In some embodiments, an implant described herein is administered to the individual in need thereof once every one to twelve months. In some embodiments, an implant described herein is administered to the individual in need thereof once every three to twelve months. In some embodiments, an implant described herein is administered to the individual in need thereof once every three to six months. In some embodiments, an implant described herein is administered to the individual in need thereof once every three months. In some embodiments, an implant described herein is administered to the individual in need thereof once every six months.

Provided in some embodiments herein is a method for decreasing central subfield thickness (CST) in an individual in need thereof. In some embodiments, the method comprises (e.g., intravitreally) administering to an eye of the individual in need thereof one or more (e.g., one or two) implant(s). In some embodiments, each implant comprising a compound having a structure represented by Formula (I): D1-L-D2. In some embodiments, D1 and D2 are each independently a steroid radical. In some embodiments, D1 and D2 are each independently a dexamethasone radical. In some embodiments, L is a linker. In some embodiments, L is a hydrolyzable linker. In some embodiments, D1, D2, and L are described elsewhere herein. In some embodiments, the one or more implant described herein collectively comprises an amount of the compound sufficient to treat the ocular disorder or condition in the eye of the individual in need thereof.

In some embodiments, the CST of each individual of a population of individuals described herein is measured at a timepoint before (e.g., a baseline measurement) and at various timepoints after an implant described herein is administered to an individual of the population of individuals. In some embodiments, a change in CST in an individual is determined by measuring the difference between a baseline CST measurement and a CST measurement measured after an implant described herein is administered to the individual. In some embodiments, the CST measurement is measured at least one week after an implant described herein is administered to the individual. In some embodiments, the CST measurement is measured at month one (M1), month two (M2), month three (M3), month four (M4), month five (M5), month six (M6), month seven (M7), month eight (M8), month nine (M9), month ten (M10), month eleven (M11), or month twelve (M12) of treatment. In some embodiments, a mean change in CST of the population of individuals is determined by averaging each change in CST of each individual at the respective timepoint. For example, a mean change in CST at month 1 can be determined by averaging each change in CST of each individual at M1, and so on until a mean change in CST at each month of treatment is determined.

In some embodiments, the population of individuals have a mean change in CST that decreases. In some embodiments, the population of individuals have a mean change in CST that decreases over a period of at least about one week. In some embodiments, the population of individuals have a mean change in CST that decreases over a period of about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, the population of individuals have a second mean change in CST measurement that decreases compared to a first CST measurement or a first mean change in CST measurement. In some embodiments, the first mean change in CST measurement for the population is compared to a CST measurement at first timepoint (e.g., baseline, at one-month of treatment, at two-months of treatment, etc.). In some embodiments, the first mean change in CST measurement for the population is at a first timepoint (e.g., at one-month of treatment, at two-months of treatment, etc.). In some embodiments, the second mean change in CST measurement for the population is at a second timepoint (at one-month of treatment, at two-months of treatment, etc.). In some embodiments, the first timepoint is before the second timepoint.

In some embodiments, CST in the eye of the individual in need thereof decreases by at least 50 micrometers (μm). In some embodiments, CST in the eye of the individual in need thereof decreases by 50 μm or more, 100 μm or more, or 150 μm or more. In some embodiments, CST in the eye of the individual in need thereof decreases by 50 μm or more, 100 μm or more, 150 μm or more, or 200 μm or more. In some embodiments, CST in the eye of the individual in need thereof decreases by 50 μm or more within about one month. In some embodiments, CST in the eye of the individual in need thereof decreases by 50 μm or more about one week after intravitreally administering the implant to the eye of the individual in need thereof. In some embodiments, CST in the eye of the individual in need thereof decreases by 50 μm or more within about one month of intravitreally administering the implant to the eye of the individual in need thereof.

In some embodiments, CST in the eye of the individual in need thereof decreases by at least 50 μm over an extended period of time. In some embodiments, CST in the eye of the individual in need thereof decreases by at least 200 μm over an extended period of time. In some embodiments, CST in the eye of the individual in need thereof decreases by about 50 μm to about 200 μm over an extended period of time. In some embodiments, CST in the eye of the individual in need thereof decreases by at least 50 μm over a period of a month or more. In some embodiments, CST in the eye of the individual in need thereof decreases by 50 μm or more, 100 μm or more, or 150 μm or more over a period of 6 months or more. In some embodiments, CST in the eye of the individual in need thereof decreases after intravitreally administering the implant to the eye of the individual in need thereof. In some embodiments, CST in the eye of the individual fluctuates from month to month.

In some embodiments, the mean change in CST decreases by about 50 μm or more after intravitreally administering the implant to the eye of each individual of the population of individuals. In some embodiments, the mean change in CST decreases by about 50 μm or more over an extended period of time, such as a month or more (e.g., 6 months or more) after intravitreally administering the implant to the eye of each individual of the population of individuals. In some embodiments, the mean change in CST decreases by about 50 μm or more, about 100 μm or more, about 150 μm or more, about 200 μm or more, about 250 μm or more.

In some embodiments, CST in the eye of the individual in need thereof decreases by at least 10%. In some embodiments, CST in the eye of the individual in need thereof decreases by 10% or more, 15% or more, 20% or more, 25% or more, 30% or more, 35% or more, or 40% or more of the baseline CST. In some embodiments, CST in the eye of the individual in need thereof decreases by 10% or more, 15% or more, 20% or more, 25% or more, 30% or more, 35% or more, 40% or more, 45% or more, 50% or more, 55% or more, 60% or more, 65% or more, 70% or more, or 75% or more, such as compared to a baseline CST (measurement). In some embodiments, CST in the eye of the individual in need thereof decreases by at least 10% over an extended period of time. In some embodiments, CST in the eye of the individual in need thereof decreases by at least 10% over a period of a month or more. In some embodiments, CST in the eye of the individual in need thereof decreases by at least 10% over a period of 6 months or more. In some embodiments, CST in the eye of the individual in need thereof decreases after intravitreally administering the implant to the eye of the individual in need thereof. In some embodiments, CST in the eye of the individual fluctuates from month to month.

In some embodiments, a mean change in CST for a population of individuals described herein decreases by at least about 10%. In some embodiments, a mean change in CST for a population of individuals described herein decreases by at least about 10% compared to a baseline CST measurement. In some embodiments, a mean change in CST for a population of individuals described herein decreases by about 10% or more, about 15% or more, about 20% or more, about 25% or more, about 30% or more, about 35% or more, about 40% or more, about 50% or more, about 60% or more, about 70% or more, or about 80% or more, such as compared to a baseline CST measurement. In some embodiments, a mean change in CST for a population of individuals described herein decreases by at least about 10% over an extended period of time. In some embodiments, a mean change in CST for a population of individuals described herein decreases by at least about 10% over an extended period of time after intravitreally administering the implant to the eye of each individual of the population of individuals. In some embodiments, the extended period of time is over a period of about 12 months.

In some embodiments, a decrease in CST is measured by a first CST measurement and a second CST measurement. In some embodiments, the second CST measurement decreases compared to the first CST measurement.

In some embodiments, the first CST measurement is a baseline CST measurement.

In some embodiments, the second CST measurement is subsequent to the first CST measurement. In some embodiments, the second CST measurement is obtained within about nine months of the first CST measurement. In some embodiments, the second CST measurement is obtained within about 9 months, within about 8 months, within about 7 months, within about 6 months, within about 5 months, within about 4 months, within about 3 months, within about 2 months, or within about 1 month of the first CST measurement.

In some embodiments, wherein CST in the eye of the individual decreases within about three months. In some embodiments, wherein CST in the eye of the individual decreases within about two months, within about one month, within about two weeks, or within about one week. In some embodiments, wherein CST in the eye of the individual decreases within about three months of intravitreally administering the implant to the eye of the individual in need thereof.

In some instances, a successful response to the implant described herein provides a decrease in CST for at least one month. In some instances, a successful response to the implant described herein provides a decrease in CST for one month or more, two months or more, three months or more, four months or more, five months or more, six months or more, seven months or more, or eight months or more. In some instances, a successful response to the implant described herein provides a decrease in CST for at least four months.

In some embodiments, CST of the individual remains decreased (e.g., a second CST measurement remains lower than a first (e.g., baseline) CST measurement) for at least about 2 months. In some embodiments, CST of the individual remains decreased (e.g., a second CST measurement remains lower than a first (e.g., baseline) CST measurement) for about 2 months or more, about 3 months or more, about 4 months or more, about 5 months or more, about 6 months or more, about 7 months or more, about 8 months or more, about 9 months or more, about 10 months or more, about 11 months or more, or about 12 months or more.

In some embodiments, the CST of the individual (or the population of individuals) does not substantially increase during a course of treatment. In some embodiments, the CST of the individual (or the population of individuals) does not substantially increase during the first three months or more, four months or more, five months or more, or six months or more of treatment. In some embodiments, the CST of the individual (or the population of individuals) does not substantially increase over a period of three months or more, four months or more, five months or more, or six months or more. In some embodiments, the individual (or the population of individuals) is administered one or two implants described herein and has RVO. In some embodiments, the CST of the individual (or the population of individuals) does not increase relative to a prior measurement (e.g., a third measurement remains higher than a second measurement) within the first three months of the implant being administered. In some embodiments, the individual (or the population of individuals) is administered one or two implants described herein and has DME. In some embodiments, the CST of the individual (or the population of individuals) does not increase relative to a prior measurement (e.g., an initial measurement (a baseline measurement, a second measurement, etc.) remains higher than a subsequent measurement (e.g., a third measurement)) within the first five months of the implant being administered.

In some embodiments, the CST of the individual (or the population of individuals) continues to decrease during a course of treatment. In some embodiments, the CST of the individual (or the population of individuals) continues to decrease during the first 6 months or more, 7 months or more, 8 months or more, 9 months or more, 10 months or more, 11 months or more, or 12 months or more of treatment. In some embodiments, the CST of the individual (or the population of individuals) continues to decrease over a period of about 6 months or more, 7 months or more, 8 months or more, 9 months or more, 10 months or more, 11 months or more, or 12 months or more. In some embodiments, the individual (or the population of individuals) is administered one or two implants described herein and has RVO. In some embodiments, the individual (or the population of individuals) is administered one or two implants described herein and has DME.

In some embodiments, an implant described herein releases a compound described herein or dexamethasone in its free form in an amount sufficient to improve vision, treat vision deterioration, treat an ocular disorder or condition, and/or decrease CST in the eye of the individual in need thereof.

In some embodiments, intraocular pressure (IOP) of the eye of the individual who has received one or more implant described herein does not significantly increase. In some embodiments, intraocular pressure (IOP) of the eye of the individual who has received one or more implant described herein does not significantly increase while the compound is released from the implant in the eye of the individual.

In some embodiments, intraocular pressure (IOP) of the eye of the individual who has received one or more implant described herein does not increase above a clinically acceptable level. In some embodiments, intraocular pressure (IOP) of the eye of the individual who has received one or more implant described herein does not increase above a clinically acceptable level while the compound is released from the implant in the eye of the individual.

In some embodiments, intraocular pressure (IOP) of the eye of the individual who has received one or more implant described herein remains at a clinically acceptable level. In some embodiments, intraocular pressure (IOP) of the eye of the individual who has received one or more implant described herein remains at a clinically acceptable level while the compound is released from the implant in the eye of the individual.

In some embodiments, intraocular pressure (IOP) of the eye of the individual who has received one or more implant described herein remains below a clinically acceptable level. In some embodiments, intraocular pressure (IOP) of the eye of the individual who has received one or more implant described herein remains below a clinically acceptable level while the compound is released from the implant in the eye of the individual.

In some embodiments, intraocular pressure (IOP) of the eye of the individual who has received one or more implant described herein does not increase above a level of about 35 millimeters of mercury (mmHg). In some embodiments, intraocular pressure (IOP) of the eye of the individual who has received one or more implant described herein does not increase above a level of about 35 mmHg while the compound is released from the implant in the eye of the individual.

In some embodiments, intraocular pressure (IOP) of the eye of the individual who has received one or more implant described herein does not increase above a level of about 25 millimeters of mercury (mmHg). In some embodiments, intraocular pressure (IOP) of the eye of the individual who has received one or more implant described herein does not increase above a level of about 25 mmHg while the compound is released from the implant in the eye of the individual.

In some embodiments, intraocular pressure (IOP) of the eye of the individual who has received one or more implant described herein remains below a level of about 35 mmHg. In some embodiments, intraocular pressure (IOP) of the eye of the individual who has received one or more implant described herein remains below a level of about 35 mmHg while the compound is released from the implant in the eye of the individual.

In some embodiments, intraocular pressure (IOP) of the eye of the individual who has received one or more implant described herein remains below a level of about 25 mmHg. In some embodiments, intraocular pressure (IOP) of the eye of the individual who has received one or more implant described herein remains below a level of about 25 mmHg while the compound is released from the implant in the eye of the individual.

In some embodiments, intraocular pressure (IOP) of the eye of the individual who has received one or more implant described herein does not increase by about 5 mmHg or more (e.g., from an initial (e.g., first) IOP measurement to a second IOP measurement, where the second IOP measurement is obtained at a time after the initial (e.g., first) IOP measurement). In some embodiments, intraocular pressure (IOP) of the eye of the individual who has received one or more implant described herein does not increase by about 10 mmHg or more (e.g., from a first IOP measurement to a second IOP measurement, where the second IOP measurement is obtained at a time after the first IOP measurement). In some embodiments, IOP of the eye of the individual who has received one or more implant described herein does not increase by more than 20 mmHg.

In some embodiments, the IOP of each individual of a population of individuals described herein is measured at a timepoint before (e.g., a baseline measurement) and at various timepoints after an implant described herein is administered to an individual of the population of individuals. In some embodiments, a change in IOP in an individual is determined by measuring the difference between a baseline IOP measurement and a IOP measurement measured after an implant described herein is administered to the individual. In some embodiments, the IOP measurement is measured at least one week after an implant described herein is administered to the individual. In some embodiments, the IOP measurement is measured at month one (M1), month two (M2), month three (M3), month four (M4), month five (M5), month six (M6), month seven (M7), month eight (M8), month nine (M9), month ten (M10), month eleven (M11), or month twelve (M12) of treatment. In some embodiments, a mean change in IOP of the population of individuals is determined by averaging each change in IOP of each individual at the respective timepoint. For example, a mean change in IOP at month 1 can be determined by averaging each change in IOP of each individual at M1, and so on until a mean change in IOP at each month of treatment is determined.

In some embodiments, the IOP of an individual of the population of individuals (or the entire population of individuals) remains substantially the same (e.g., the change in IOP is does not change (i.e., increase or decrease) by more than about 15 millimeters of mercury (mmHg)) as a baseline measurement. In some embodiments, the IOP of an individual of the population of individuals (or the entire population of individuals) remains substantially the same as a baseline measurement while the compound is released from the one or more implant (in the eye(s) of the individual(s)). In some embodiments, the IOP of an individual of the population of individuals (or the entire population of individuals) remains substantially the same as a baseline measurement during a course of treatment. In some embodiments, the IOP of an individual of the population of individuals (or the entire population of individuals) remains substantially the same as a baseline measurement over a period of at least about one week. In some embodiments, the IOP of an individual of the population of individuals (or the entire population of individuals) remains substantially the same as a baseline measurement over a period of about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more. In some embodiments, the population of individuals have a second mean change in IOP measurement that remains substantially the same as a first mean change in IOP measurement. In some embodiments, the first mean change in IOP measurement for the population is compared to an IOP measurement at first timepoint (e.g., baseline, at one-month of treatment, at two-months of treatment, etc.). In some embodiments, the first mean change in IOP measurement for the population is at a first timepoint (e.g., at one-month of treatment, at two-months of treatment, etc.). In some embodiments, the second mean change in IOP measurement for the population is at a second timepoint (at one-month of treatment, at two-months of treatment, etc.). In some embodiments, the first timepoint is before the second timepoint. In some embodiments, the IOP of an individual of the population of individuals (or the entire population of individuals) remains below a level of 35 mmHg during a course of treatment. In some embodiments, the IOP of an individual of the population of individuals (or the entire population of individuals) remains below a level of 25 mmHg during a course of treatment.

In some embodiments, the amount of the compound administered to the individual in need thereof is sufficient to improve vision, treat vision deterioration, treat an ocular disorder or condition, and/or decrease CST in the eye of the individual in need thereof while avoiding an increase in intraocular pressure (IOP) in the individual in need thereof. In some embodiments, the amount of the compound administered to the individual in need thereof is sufficient to improve vision, treat vision deterioration, treat an ocular disorder or condition, and/or decrease CST in the eye of the individual in need thereof while avoiding a clinically significant increase in intraocular pressure (IOP) in the individual in need thereof. In some embodiments, the amount of the compound administered to the individual in need thereof is sufficient to improve vision, treat vision deterioration, treat an ocular disorder or condition, and/or decrease CST in the eye of the individual in need thereof while avoiding an increase in intraocular pressure (IOP) above a level of 25 millimeters of mercury (mmHg) in the individual in need thereof. In some embodiments, the IOP of an eye of an individual (e.g., described herein) remains within acceptable levels (e.g., below a level of 25 mmHg), such as after administration of one or more implant provided herein into an eye of an individual (e.g., described herein). In some embodiments, the IOP of the individual does not increase (e.g., above a level of 25 mmHg), such as after administration of one or more implant provided herein into an eye of an individual (e.g., described herein). In some embodiments, the IOP of the individual decreases, such as after administration of one or more implant provided herein into an eye of an individual (e.g., described herein).

In some embodiments, a population of individuals administered one or more implant described herein, such as a Compound 1 IVT implant, have a mean change in CST that decreases over a period of at least about one week. In some embodiments, the population of individuals administered one or more implant described herein, such as a Compound 1 IVT implant, have a mean change in CST that decreases over a period of at least about one week, about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more.

In some embodiments, the mean change in CST in the population of individuals decreases by about 50 μm or more over an extended period of time, such as for a month or more after intravitreally administering the implant to the eye of each individual in the population of individuals in need thereof. In some embodiments, the mean change in CST in the population of individuals decreases by about 50 μm or more, by about 100 μm or more, about 150 μm or more, about 200 μm or more, or about 250 μm or more. In some embodiments, the mean change in CST decreases by about 50 μm or more over an extended period of time, such as for a month or more, 2 months or more, 3 months or more, 4 months or more, 5 months or more, or 6 months or more after intravitreally administering the implant to the eye of each individual in the population of individuals in need thereof.

In some embodiments, the mean change in CST decreases by at least about 10% compared to the baseline CST over an extended period of time after administering the implant to the eye of each individual of the population of individuals in need thereof. In some embodiments, the mean change in CST decreases by at least about 10% compared to the baseline CST over an extended period of time after intravitreally administering the implant to the eye of each individual of the population of individuals in need thereof. In some embodiments, the mean change in CST decreases by at least about 10% or more, about 15% or more, about 20% or more, about 25% or more, about 30% or more, about 35% or more, about 40% or more, about 50% or more, about 60% or more, about 70% or more, or about 80% or more compared to baseline CST over an extended period of time after intravitreally administering the implant to the eye of each individual of the population of individuals in need thereof. In some embodiments, the mean change in CST decreases by at least about 10% compared to the baseline CST over an extended period of time after intravitreally administering the implant to the eye of each individual of the population of individuals in need thereof compared to the baseline CST for a month or more after administering the implant to the eye of each individual of the population of individuals in need thereof. In some embodiments, the mean change in CST decreases by at least about 10% compared to the baseline CST over an extended period of time after intravitreally administering the implant to the eye of each individual of the population of individuals in need thereof compared to the baseline CST for a month or more, 2 months or more, 3 months or more, 4 months or more, 5 months or more, or 6 months or more after administering the implant to the eye of each individual of the population of individuals in need thereof.

In some embodiments, a population of individuals with diabetic macular edema (DME) administered one or more implant described herein, such as a Compound 1 IVT implant, have a mean change in CST that decreases over a period of at least about 1 month or more. In some embodiments, the population of individuals with DME is administered one implant described herein, such as a Compound 1 IVT implant. In some embodiments, the population of individuals with DME is administered two implants described herein, such as a Compound 1 IVT implant.

In some embodiments, a population of individuals with DME is administer two implants and the population has a mean change in CST that decreases by about 60 μm or more over an extended period of time, such as over a period of at least about 1 month or more. In some embodiments, the mean change in CST in the population of individuals with DME administered two implants decreases by about 60 μm or more, about 80 μm or more, about 100 μm or more, or about 120 μm or more over an extended period of time, such as over a period of at least about 1 month or more. In some embodiments, the mean change in CST in the population of individuals with DME administered two implants decreases by about 60 μm or more over an extended period of time, such as over a period of at least about 1 month or more, about 2 months or more, about 3 months or more, about 4 months or more, or about 5 months or more.

In some embodiments, a population of individuals with DME is administered one implant and the population has a mean change in CST that decreases by about 40 μm or more over an extended period of time, such as over a period of at least about 1 month or more. In some embodiments, the mean change in CST in the population of individuals with DME administered one implant decreases by about 40 μm or more, 60 μm or more, about 80 μm or more, or about 100 μm or more over an extended period of time, such as over a period of at least about 1 month or more. In some embodiments, the mean change in CST in the population of individuals with DME administered one implant decreases by about 40 μm or more over an extended period of time, such as over a period of at least about 1 month or more, about 2 months or more, about 3 months or more, about 4 months or more, or about 5 months or more.

In some embodiments, a population of individuals with retinal vein occlusion (RVO) administered one or more implant described herein, such as a Compound 1 IVT implant, have a mean change in CST that decreases over a period of at least about 1 month or more. In some embodiments, the population of individuals with RVO is administered one implant described herein, such as a Compound 1 IVT implant. In some embodiments, the population of individuals with RVO is administered two implants described herein, such as a Compound 1 IVT implant.

In some embodiments, a population of individuals with RVO is administered two implants and the population has a mean change in CST that decreases by about 100 μm or more over an extended period of time, such as over a period of at least about 1 month or more. In some embodiments, the mean change in CST in the population of individuals with RVO administered two implants decreases by about 100 μm or more, about 150 μm or more, or about 200 μm or more over an extended period of time, such as over a period of at least about 1 month or more. In some embodiments, the mean change in CST in the population of individuals with RVO administered two implants decreases by about 100 μm or more over an extended period of time, such as over a period of at least about 1 month or more, about 2 months or more, about 3 months or more, about 4 months or more, about 5 months or more, or about 6 months or more.

In some embodiments, a population of individuals with RVO is administered one implant and the population has a mean change in CST that decreases by about 150 μm or more over an extended period of time, such as over a period of at least about 1 month or more. In some embodiments, the mean change in CST in the population of individuals with RVO administered one implant decreases by about 150 μm or more, about 200 μm or more, or about 250 μm or more over an extended period of time, such as over a period of at least about 1 month or more. In some embodiments, the mean change in CST in the population of individuals with RVO administered one implant decreases by about 150 μm or more over an extended period of time, such as over a period of at least about 1 month or more, about 2 months or more, about 3 months or more, about 4 months or more, about 5 months or more, or about 6 months or more.

In some embodiments, a population of individuals administered one or more implant described herein, such as a Compound 1 IVT implant, have a mean change in vision (improvement) that increases, such as over a period of about one week. In some embodiments, a population of individuals administered one or more implant described herein, such as a Compound 1 IVT implant, have a mean change in vision (improvement) as measured by one or more ETDRS letter test that increases, such as over a period of about one week.

In some embodiments, a population of individuals administered one or more implant described herein, such as a Compound 1 IVT implant, have a mean change in vision (improvement) that increases over a period of at least about one week. In some embodiments, the population of individuals administered one or more implant described herein, such as a Compound 1 IVT implant, have a mean change in vision (improvement) that increases over a period of at least about one week, about 1 month or more, about 2 months or more, about 3 months or more, about 4 months or more, about 5 months or more, or about 6 months or more.

In some embodiments, vision of a population of individuals administered one or more implant described herein, such as a Compound 1 IVT implant improves. In some embodiments, vision of a population of individuals administered one or more implant described herein, such as a Compound 1 IVT implant, as measured by ETDRS letter test, improves. In some embodiments, the vision of the population of individuals improves, such as by at least 5 letters by BCVA. In some embodiments, the vision of the population of individuals as measured by one or more ETDRS letter test improves, such as by at least 5 letters by BCVA. In some embodiments, the vision of the population of individuals improves, such as by at least 5 letters, 5 or more letters, 6 or more letters, 7 or more letters, or 8 or more letters by BCVA.

In some embodiments, a population of individuals with diabetic macular edema (DME) or retinal vein occlusion (RVO) administered one or more implant described herein, such as a Compound 1 IVT implant, have a mean change in vision (improvement) of at least 5 letters by BCVA, such as over a period of at least about 1 month or more. In some embodiments, a population of individuals with DME or RVO administered one or two implant described herein, such as a Compound 1 IVT implant, have a mean change in vision (improvement) of at least 5 letters by BCVA over a period of at least about 1 month or more. In some embodiments, the population of individuals with DME or RVO is administered one implant described herein, such as a Compound 1 IVT implant. In some embodiments, a population of individuals with DME or RVO administered one or two implants have a mean change in vision (improvement) of at least 5 letters by BCVA over a period of at least about 1 month or more. In some embodiments, a population of individuals with DME or RVO administered one or two implant have a mean change in vision (improvement) of at least 5 letters by BCVA over a period of at least about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more.

In some embodiments, a population of individuals administered one or more implant described herein, such as a Compound 1 IVT implant, have a change in intraocular pressure (IOP) of no more than 15 millimeters of mercury (mmHg). In some embodiments, a population of individuals administered one or more implant described herein, such as an Compound 1 IVT implant, have a change in intraocular pressure (IOP) of no more than 15 millimeters of mercury (mmHg) compared to baseline. In some embodiments, a population of individuals administered one or more implant described herein, such as a Compound 1 IVT implant, have a change in intraocular pressure (IOP) of no more than 15 millimeters of mercury (mmHg) over a period of at least about one week. In some embodiments, a population of individuals administered one or more implant described herein, such as a Compound 1 IVT implant, have a change in intraocular pressure (IOP) of no more than 15 millimeters of mercury (mmHg) over a period of about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more.

In some embodiments, a population of individuals with RVO administered one or more implant described herein, such as a Compound 1 IVT implant, have a change in IOP of no more than about 10 mmHg (compared to baseline), such as over a period of at least about one week. In some embodiments, a population of individuals with RVO administered one or two implants have a change in IOP of no more than about 10 mmHg compared to baseline, such as over a period of at least about one week. In some embodiments, a population of individuals with RVO administered one or two implants have a change in IOP of no more than about 10 mmHg while the compound is released from the implant(s) in the one or more eye of each individual. In some embodiments, a population of individuals with RVO administered one or two implants have a change in IOP of no more than about 10 mmHg (compared to baseline) over a period of about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more.

In some embodiments, a population of individuals with DME administered one or more implant described herein, such as a Compound 1 IVT implant, have a change in IOP of no more than about 5 mmHg (compared to baseline), such as over a period of at least about one week. In some embodiments, a population of individuals with DME administered one or two implants have a change in IOP of no more than about 5 mmHg compared to baseline, such as over a period of at least about one week. In some embodiments, a population of individuals with DME administered one or two implants have a change in IOP of no more than about 5 mmHg while the compound is released from the implant(s) in the one or more eye of each individual. In some embodiments, a population of individuals with DME administered one or two implants have a change in IOP of no more than about 5 mmHg (compared to baseline) over a period of about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more.

In some embodiments, IOP of an eye of an individual (of the population of individuals) does not increase above a level of about 35 mmHg. In some embodiments, IOP of an eye of an individual (of the population of individuals) does not increase above a level of about 35 mmHg while the compound is released from the implant in the eye of the individual. In some instances, IOP of an eye of an individual (of the population of individuals) is maintained below a level of 35 mmHg.

In some embodiments, IOP of an eye of an individual (of the population of individuals) does not increase above a level of about 30 mmHg. In some embodiments, IOP of an eye of an individual (of the population of individuals) does not increase above a level of about 30 mmHg while the compound is released from the implant in the eye of the individual. In some instances, IOP of an eye of an individual (of the population of individuals) is maintained below a level of 30 mmHg.

In some embodiments, IOP of an eye of an individual (of the population of individuals) does not increase above a level of about 25 mmHg. In some embodiments, IOP of an eye of an individual (of the population of individuals) does not increase above a level of about 25 mmHg while the compound is released from the implant in the eye of the individual. In some instances, IOP of an eye of an individual (of the population of individuals) is maintained below a level of 25 mmHg.

In some embodiments, IOP of an eye of an individual (of the population of individuals) does not increase above a level of about 20 mmHg. In some embodiments, IOP of an eye of an individual (of the population of individuals) does not increase above a level of about 20 mmHg while the compound is released from the implant in the eye of the individual. In some instances, IOP of an eye of an individual (of the population of individuals) is maintained below a level of 20 mmHg.

In some embodiments, IOP of an eye of an individual of the population of individuals does not have a change in IOP of greater than or equal to 15 mmHg. In some embodiments, IOP of an eye of an individual of the population of individuals does not increase or decrease by more than about 15 mmHg while the compound is release from the implant in the eye of the individual. In some instances, a change in IOP of an eye of an individual of the population of individuals remains within 15 mmHg of a baseline measurement in IOP.

In some embodiments, IOP of an eye of an individual of the population of individuals does not have a change in IOP of greater than or equal to 10 mmHg. In some embodiments, IOP of an eye of an individual of the population of individuals does not increase or decrease by more than about 10 mmHg while the compound is release from the implant in the eye of the individual. In some instances, a change in IOP of an eye of an individual of the population of individuals remains within 10 mmHg of a baseline measurement in IOP.

In some embodiments, IOP of an eye of an individual of the population of individuals does not have a change in IOP of greater than or equal to 5 mmHg. In some embodiments, IOP of an eye of an individual of the population of individuals does not increase or decrease by more than about 5 mmHg while the compound is release from the implant in the eye of the individual. In some instances, a change in IOP of an eye of an individual of the population of individuals remains within 5 mmHg of a baseline measurement in IOP.

In some embodiments, an individual (of the population of individuals) described herein, such as administered one or more implant described herein (e.g., a Compound 1 IVT implant), does not require an intervention, retreatment, or rescue therapy. In some embodiments, an individual (of the population of individuals) described herein, such as administered one or more implant described herein (e.g., a Compound 1 IVT implant), does not require an IOP-lowering medication. In some embodiments, an individual (of the population of individuals) described herein, such as administered one or more implant described herein (e.g., a Compound 1 IVT implant), does not require a procedural intervention. In some embodiments, an individual (of the population of individuals) described herein, such as administered one or more implant described herein (e.g., a Compound 1 IVT implant), does not require a surgical intervention.

In some instances, an individual (of the population of individuals) described herein (e.g., an individual with RVO), such as administered Implant A, does require IOP-lowering medication (see Haller et al.), whereas when the individual (of the population of individuals) is administered an implant described herein, such as a Compound 1 IVT implant, the individual (or population thereof) does not require IOP-lowering medication. In some instances, an individual (of the population of individuals) described herein (e.g., an individual with RVO), such as administered Implant A, does require a procedural intervention (see Haller et al.), whereas when the individual (of the population of individuals) is administered an implant described herein, such as a Compound 1 IVT implant, the individual (or population thereof) does not require a procedural intervention.

In some instances, an individual (of the population of individuals) described herein (e.g., an individual with DME), such as administered Implant A, does require IOP-lowering medication (see Boyer et al.). In some instances, an individual (of the population of individuals) described herein (e.g., an individual with DME), such as administered Implant A, does require a procedural intervention (see Boyer et al.), whereas when the individual (of the population of individuals) is administered an implant described herein, such as a Compound 1 IVT implant, the individual (or population thereof) does not require a procedural intervention. In some instances, an individual (of the population of individuals) described herein (e.g., an individual with DME), such as administered Implant A, does require a surgical intervention (see Boyer et al.), whereas when the individual (of the population of individuals) is administered an implant described herein, such as a Compound 1 IVT implant, the individual (or population thereof) does not require a surgical intervention.

In some embodiments, the amount of a compound described herein (e.g., Compound 1) sufficient to improve vision, treat vision deterioration, treat an ocular disorder or condition, and/or decrease CST in the eye of the individual in need thereof is less than about 500 micrograms (μg). In some embodiments, the amount of a compound described herein (e.g., Compound 1) sufficient to improve vision, treat vision deterioration, treat an ocular disorder or condition, and/or decrease CST in the eye of the individual in need thereof is 500 μg or less, 400 μg or less, 300 μg or less, 200 μg or less, 100 μg or less, or 50 μg or less. In some embodiments, the amount of a compound described herein (e.g., Compound 1) sufficient to improve vision, treat vision deterioration, treat an ocular disorder or condition, and/or decrease CST in the eye of the individual in need thereof is 250 μg or less, 150 μg or less, or 50 μg or less. In some embodiments, the amount of a compound described herein (e.g., Compound 1) sufficient to improve vision, treat vision deterioration, treat an ocular disorder or condition, and/or decrease CST in the eye of the individual in need thereof is about 250 μg or less. In some embodiments, the amount of a compound described herein (e.g., Compound 1) sufficient to improve vision, treat vision deterioration, treat an ocular disorder or condition, and/or decrease CST in the eye of the individual in need thereof is about 175 μg or less. In some embodiments, the amount of a compound described herein (e.g., Compound 1) sufficient to improve vision, treat vision deterioration, treat an ocular disorder or condition, and/or decrease CST in the eye of the individual in need thereof is about 156 μg or less. In some embodiments, the amount of a compound described herein (e.g., Compound 1) sufficient to improve vision, treat vision deterioration, treat an ocular disorder or condition, and/or decrease CST in the eye of the individual in need thereof is about 150 μg or less. In some embodiments, the amount of a compound described herein (e.g., Compound 1) sufficient to improve vision, treat vision deterioration, treat an ocular disorder or condition, and/or decrease CST in the eye of the individual in need thereof is about 100 μg or less.

In some embodiments, the implant is intravitreally administered to the eye of the individual in need thereof in an amount of about 10 μg to about 500 μg of the compound. In some embodiments, the implant is intravitreally administered to the eye of the individual in need thereof in an amount of about 250 μg or less of the compound. In some embodiments, the implant is intravitreally administered to the eye of the individual in need thereof in an amount of less than about 175 μg of the compound. In some embodiments, the implant is intravitreally administered to the eye of the individual in need thereof in an amount of about 100 μg or less of the compound.

In some embodiments, the implant is intravitreally administered to the eye of the individual in need thereof in an amount of about 75 μg to about 175 μg of the compound. In some embodiments, the implant is intravitreally administered to the eye of the individual in need thereof in an amount of about 50 μg to about 110 μg of the compound. In some embodiments, the implant is intravitreally administered to the eye of the individual in need thereof in an amount of about 78 μg to about 106 μg of the compound.

In some embodiments, the amount of a compound described herein (e.g., Compound 1) sufficient to improve vision, treat vision deterioration, treat an ocular disorder or condition, and/or decrease CST in the eye of the individual in need thereof is at least about 10 micrograms (μg). In some embodiments, the amount of a compound described herein (e.g., Compound 1) sufficient to improve vision, treat vision deterioration, treat an ocular disorder or condition, and/or decrease CST in the eye of the individual in need thereof is 10 μg or more, 100 μg or more, 200 μg or more, 300 μg or more, 400 μg or more, or 500 μg or more. In some embodiments, the amount of a compound described herein (e.g., Compound 1) sufficient to improve vision, treat vision deterioration, treat an ocular disorder or condition, and/or decrease CST in the eye of the individual in need thereof is 10 μg or more, 50 μg or more, or 100 μg or more. In some embodiments, the amount of a compound described herein (e.g., Compound 1) sufficient to improve vision, treat vision deterioration, treat an ocular disorder or condition, and/or decrease CST in the eye of the individual in need thereof is about 100 μg or more.

In some embodiments, the amount of a compound described herein (e.g., Compound 1) sufficient to improve vision, treat vision deterioration, treat an ocular disorder or condition, and/or decrease CST in the eye of the individual in need thereof is about 10 μg to about 500 μg. In some embodiments, the amount of a compound described herein (e.g., Compound 1) sufficient to improve vision, treat vision deterioration, treat an ocular disorder or condition, and/or decrease CST in the eye of the individual in need thereof is about 50 μg to about 250 μg. In some embodiments, the amount of a compound described herein (e.g., Compound 1) sufficient to improve vision, treat vision deterioration, treat an ocular disorder or condition, and/or decrease CST in the eye of the individual in need thereof is about 75 to about 175 μg. In some embodiments, the amount of a compound described herein (e.g., Compound 1) sufficient to improve vision, treat vision deterioration, treat an ocular disorder or condition, and/or decrease CST in the eye of the individual in need thereof is about 50 to about 110 μg. In some embodiments, the amount of a compound described herein (e.g., Compound 1) sufficient to improve vision, treat vision deterioration, treat an ocular disorder or condition, and/or decrease CST in the eye of the individual in need thereof is about 75 to about 110 μg. In some embodiments, the amount of a compound described herein (e.g., Compound 1) sufficient to improve vision, treat vision deterioration, treat an ocular disorder or condition, and/or decrease CST in the eye of the individual in need thereof is about 78 to about 106 μg.

In some embodiments, the amount of a compound described herein (e.g., Compound 1) sufficient to improve vision, treat vision deterioration, treat an ocular disorder or condition, and/or decrease CST in the eye of the individual in need thereof is about 92 μg. In some embodiments, the amount of Compound 1 sufficient to improve vision, treat vision deterioration, treat an ocular disorder or condition, and/or decrease CST in the eye of the individual in need thereof is about 92 μg.

In some embodiments, an amount of dexamethasone by free (e.g., free form) weight sufficient to improve vision, treat vision deterioration, treat an ocular disorder or condition, and/or decrease CST in the eye of the individual in need thereof is at most about 500 micrograms (μg). In some embodiments, an amount of dexamethasone by free (e.g., freeform) weight sufficient to improve vision, treat vision deterioration, treat an ocular disorder or condition, and/or decrease CST in the eye of the individual in need thereof is 500 μg or less, 400 μg or less, 300 μg or less, 200 μg or less, 100 μg or less, or 50 μg or less. In some embodiments, an amount of dexamethasone by free (e.g., free form) weight sufficient to improve vision, treat vision deterioration, treat an ocular disorder or condition, and/or decrease CST in the eye of the individual in need thereof is 200 μg or less, 100 μg or less, or 50 μg or less. In some embodiments, an amount of dexamethasone by free (e.g., free form) weight sufficient to improve vision, treat vision deterioration, treat an ocular disorder or condition, and/or decrease CST in the eye of the individual in need thereof is about 200 μg or less. In some embodiments, an amount of dexamethasone by free (e.g., free form) weight sufficient to improve vision, treat vision deterioration, treat an ocular disorder or condition, and/or decrease CST in the eye of the individual in need thereof is about 100 μg or less.

In some embodiments, an amount of dexamethasone by free (e.g., free form) weight sufficient to improve vision, treat vision deterioration, treat an ocular disorder or condition, and/or decrease CST in the eye of the individual in need thereof is at least about 10 micrograms (μg). In some embodiments, an amount of dexamethasone by free (e.g., free form) weight sufficient to improve vision, treat vision deterioration, treat an ocular disorder or condition, and/or decrease CST in the eye of the individual in need thereof is 10 μg or more, 100 μg or more, 200 μg or more, 300 μg or more, 400 μg or more, or 500 μg or more. In some embodiments, an amount of dexamethasone by free (e.g., free form) weight sufficient to improve vision, treat vision deterioration, treat an ocular disorder or condition, and/or decrease CST in the eye of the individual in need thereof is 10 μg or more, 50 μg or more, or 100 μg or more. In some embodiments, an amount of dexamethasone by free (e.g., free form) weight sufficient to improve vision, treat vision deterioration, treat an ocular disorder or condition, and/or decrease CST in the eye of the individual in need thereof is about 50 μg or more.

In some embodiments, an amount of dexamethasone by free (e.g., free form) weight sufficient to improve vision, treat vision deterioration, treat an ocular disorder or condition, and/or decrease CST in the eye of the individual in need thereof is about 10 μg to about 500 μg. In some embodiments, an amount of dexamethasone by free (e.g., free form) weight sufficient to improve vision, treat vision deterioration, treat an ocular disorder or condition, and/or decrease CST in the eye of the individual in need thereof is about 50 μg to about 200 μg. I In some embodiments, an amount of dexamethasone by free (e.g., free form) weight sufficient to improve vision, treat vision deterioration, treat an ocular disorder or condition, and/or decrease CST in the eye of the individual in need thereof is about 50 μg to about 100 μg.

Provided herein in some embodiments herein is an implant comprising a compound having a structure described herein in an amount described herein. In some instances, the compound has a structure of D1-L-D2. In some instances, D1 and D2 are each independently a steroid (e.g., dexamethasone) radical, such as a steroid described herein. In some instances L is a linker, such as a linker described herein, that connects D1 and D2 (e.g., such that the linker is configured to release D1 and D2 in their free form in an eye of an individual in need thereof). In some instances, the compound is Compound 1.

In some instances, one or more implant described herein, such as a Compound 1 IVT implant, is administered to an individual in need thereof, such as an individual diagnosed with diabetic macular edema (DME). In some instances, the individual in need thereof has received a previous treatment (for DME), such as anti-VEGF therapeutics, prior to receiving the Compound 1 IVT implant. In some instances, one implant described herein, such as a Compound 1 IVT implant, is administered to an individual in need thereof. In some instances, two implants described herein, such as a Compound 1 IVT implant, are (sequentially, such as, in series or one month or more after receiving the first implant dose) administered to an individual in need thereof. In some instances, a first implant dose is administered and then a second (e.g., retreatment) implant dose is administered, such as one month or more after receiving the first implant dose.

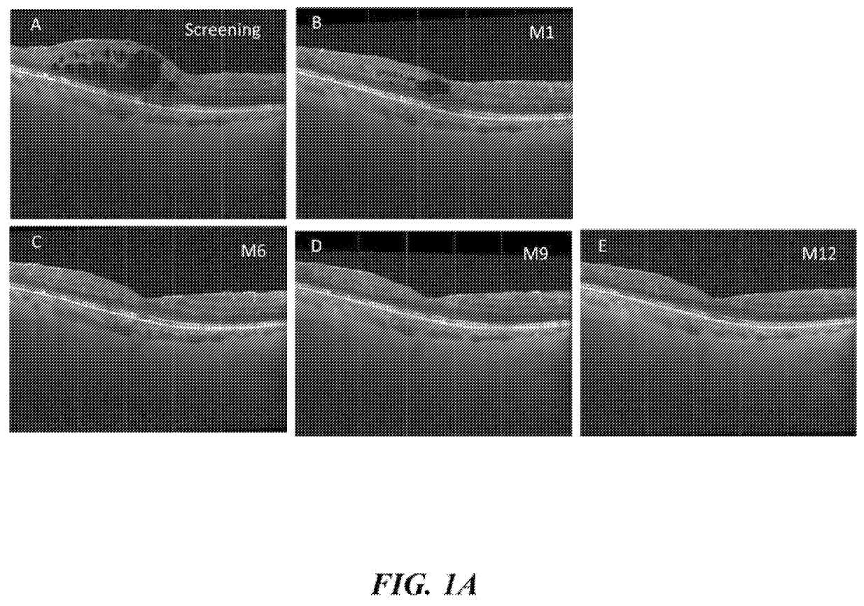

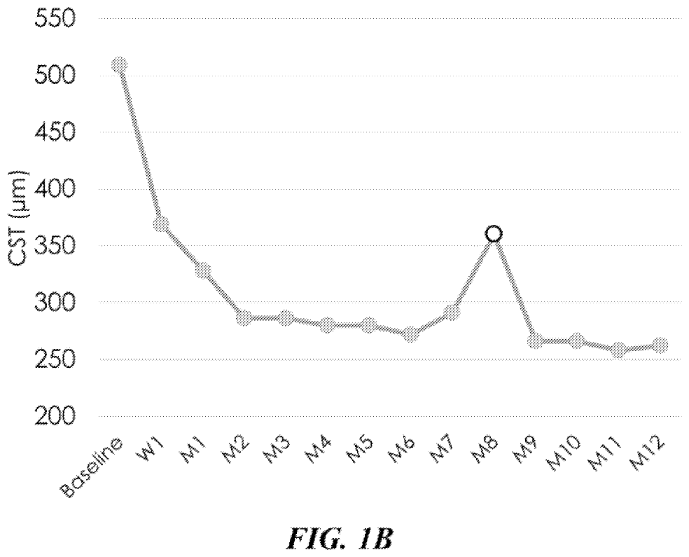

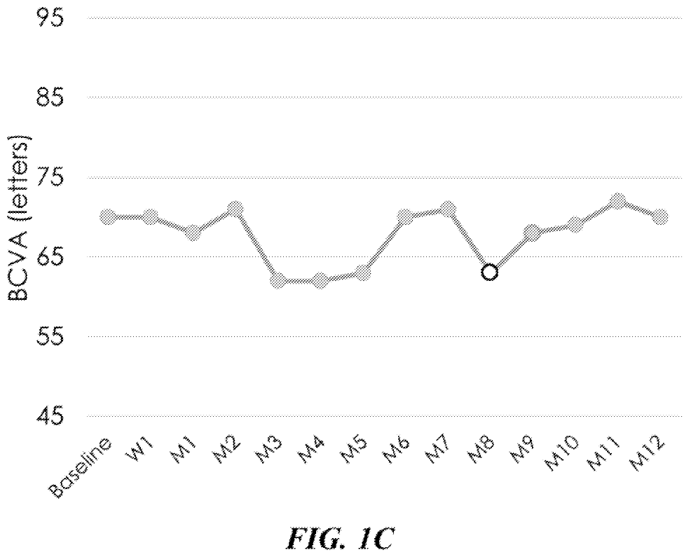

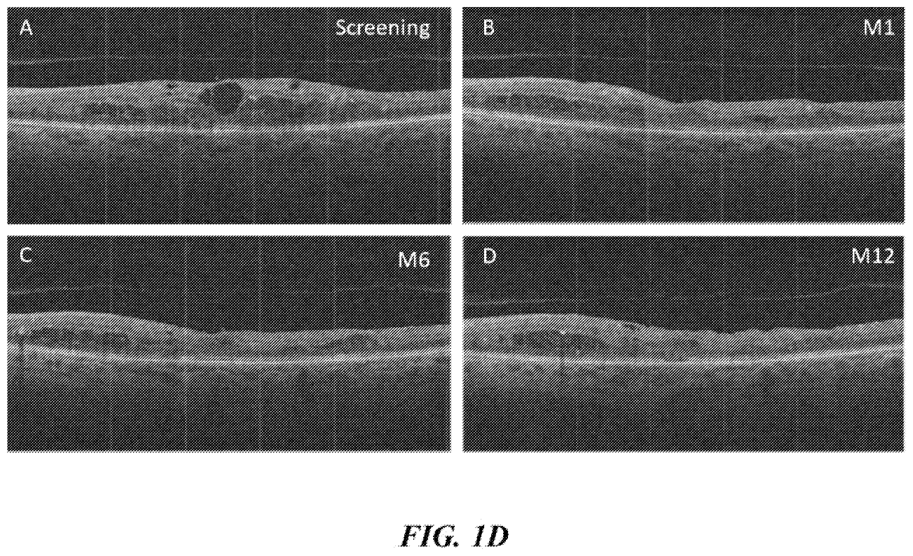

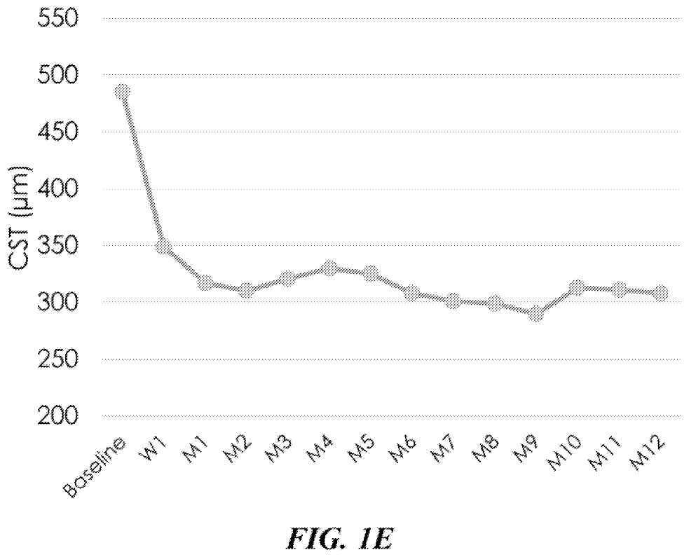

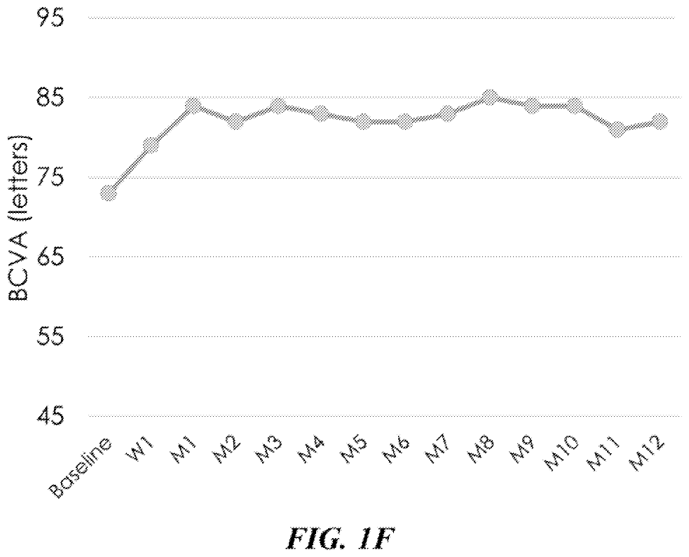

In some instances, a single Compound 1 IVT implant is administered to (e.g., at least 10) individuals diagnosed with DME and a number of those individuals, such as at least 50% (e.g., 50% or more, 75% or more, or 90% or more) of those individuals, respond (favorably) to the Compound 1 IVT implant (e.g., see FIGS. 1A-1F).

FIG. 1A-1C is representative of a responder (profile), such as a responder profile for an individual that has received a previous treatment for DME (e.g., anti-VEGF therapeutics) prior to receiving the Compound 1 IVT implant. Panel A-E of FIG. 1A show the edema of the responder at baseline/screening (BCVA is 64 letters, CST is 509 μm), 1 month (M1) after an initial implantation of the Compound 1 IVT implant (BCVA is 68 letters, CST is 328 μm), 6 Mo after an initial implantation of the Compound 1 IVT implant (BCVA is about 71 letters, CST is 272 μm), 9 Mo after an initial implantation of the Compound 1 IVT implant (BCVA is 68 letters, CST is 266 μm), and 12 Mo after an initial implantation of the Compound 1 IVT implant (BCVA is about 70 letters, CST is about 260 μm). In some instances, such as within one-week of implantation of the Compound 1 IVT implant, the responder has a (rapid) decrease in CST (−140 μm (about a 30% decrease from baseline CST); 509 μm to 369 μm—see FIG. 1A and FIG. 1). In some instances, such as out to month 2 (M2), the responder has a strong decline in CST, such as, where CST reaches 286 μm (about a 45% decrease from baseline CST) by M2 (FIG. 1). In some instances, such as from month 2 (M2) to month 6 (M6), the CST level of the responder is maintained (e.g., at 272 μm—see FIG. 1B and FIG. 1C), such as, demonstrating the durability of the Compound 1 IVT implant. In some instances, such as from months 3-5 (M3-M5), the responder has small fluctuations in visual acuity (e.g., as measured by BCVA—see FIG. 1C). In some instances, the responder has a maintained BCVA of about 70 letters (FIG. 1C). In some instance, such as at month 8 (M8), the responder has a recurrence of edema (e.g., 360 μm) and/or a loss of letters (e.g., 63 letters) occurs (see FIG. 1B and FIG. 1C). In some instances, the responder receives a retreatment dose (open circle of FIG. 1A and FIG. 1B), such as a second Compound 1 IVT implant. In some instances, such as after retreatment, the responder continues to respond well (e.g., out to month 10 (M10)) to Compound 1 IVT implant, such as having a decrease in CST to lowest achieved thickness (e.g., of 266 μm (about a 50% decrease from baseline CST)) and/or vision improvement (e.g., to 69 letters) (see FIGS. 1A-1C). In some instances, such as after retreatment, the responder continues to respond well (e.g., out to month 11 (M11)) to Compound 1 IVT implant, such as having a decrease in CST to lowest achieved thickness (e.g., of 258 μm (about a 50% decrease from screening CST)) and/or vision improvement (e.g., to 72 letters) (see FIGS. 1A-1C).

FIG. 1D-1F is representative of a responder (profile), such as a responder profile for an individual that has received a previous treatment for DME (e.g., anti-VEGF therapeutics) prior to receiving the Compound 1 IVT implant. Panel A-D of FIG. 1D show the edema of the responder at baseline/screening (BCVA is 73 letters, CST is 485 μm), 1 month (M1) after an initial implantation of the Compound 1 IVT implant (BCVA is 84 letters, CST is 317 μm), 6 Mo after an initial implantation of the Compound 1 IVT implant (BCVA is about 80 letters, CST is 310 μm), and 12 Mo after an initial implantation of the Compound 1 IVT implant (BCVA is 82 letters, CST is 308 μm). In some instances, such as within one-week of implantation of the Compound 1 IVT implant, the responder has a (rapid) decrease in CST (−136 μm (about a 25% decrease from baseline CST); 485 μm to 317 μm—see FIG. 1D and FIG. 1E). In some instances, such as out to month 2 (M2), the responder has a strong decline in CST, such as, where CST reaches 310 μm (about a 35% decrease from baseline CST) by M2 (FIG. 1E). In some instances, such as from month 2 (M2) to month 6 (M6), the CST level of the responder is maintained (e.g., at 308 μm—see FIG. 1D and FIG. 1E), such as, demonstrating the durability of the Compound 1 IVT implant. In some instances, the responder has a maintained BCVA of about 80 letters (FIG. 1F).

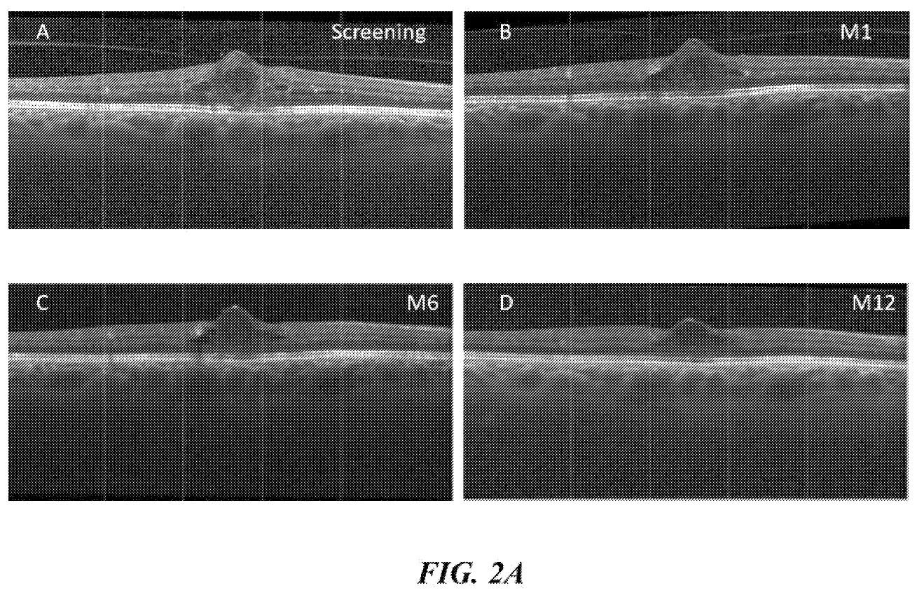

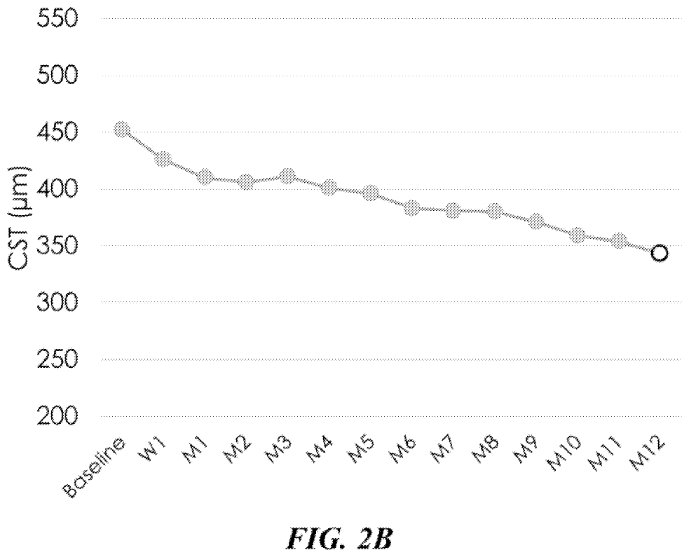

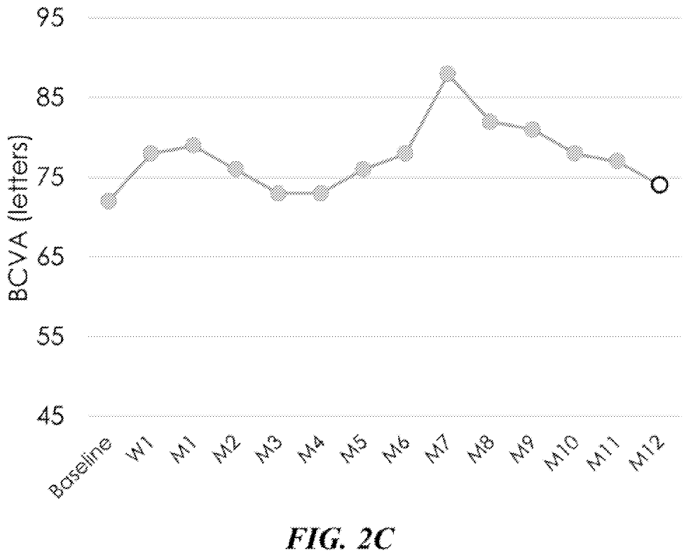

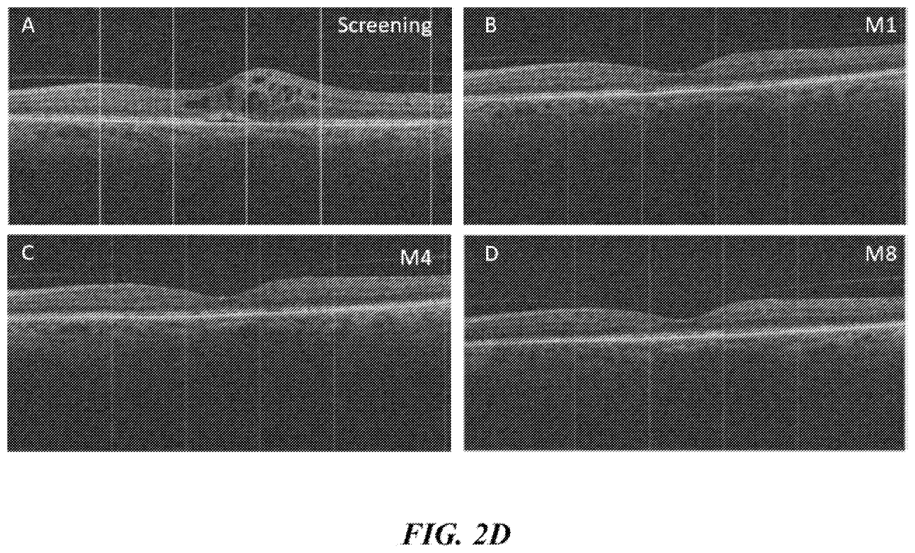

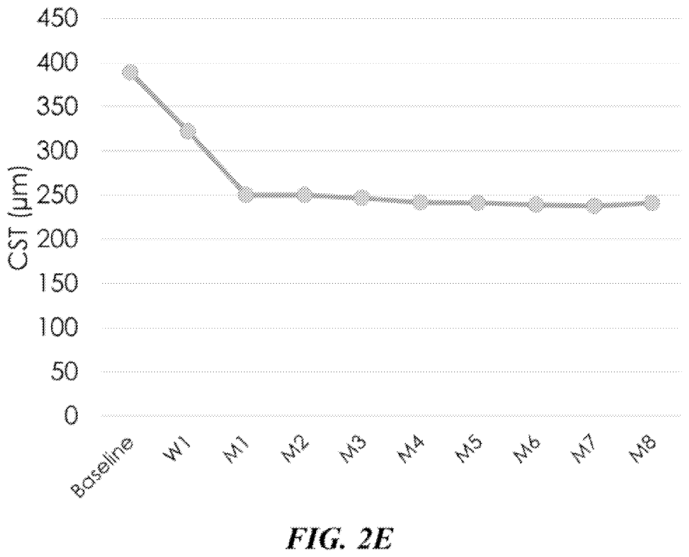

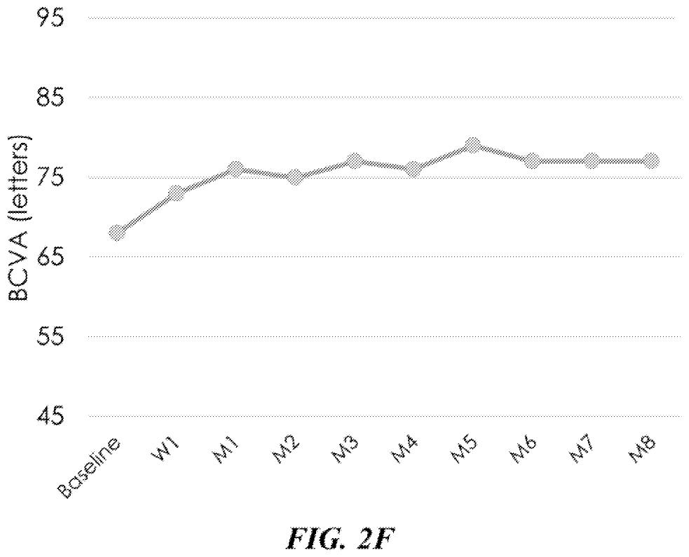

In some instances, two Compound 1 IVT implants are administered to (e.g., at least 5) individuals diagnosed with DME and a number of those individuals, such as at least 50% (e.g., 50% or more, 75% or more, or 90% or more) of those individuals, respond (favorably) to the Compound 1 IVT implants (e.g., see FIGS. 2A-2F).

FIG. 2A-2C is representative of a responder (profile), such as a responder profile for treatment naïve individual receiving two Compound 1 IVT implants. Panel A-D of FIG. 2A show the edema of the responder at baseline/screening (BCVA is 72 letters, CST is 452 μm), 1 month (M1) after an initial implantation of the Compound 1 IVT implant (BCVA is 79 letters, CST is 410 μm), 6 months (M6) after an initial implantation of the Compound 1 IVT implant (BCVA is about 80 letters, CST is about 383 μm), and 12 months (M12) after an initial implantation of the Compound 1 IVT implant (BCVA is about 75 letters, CST is 343 μm). In some instances, the responder has a BCVA of 73 letters and a CST of 411 μm 3 Mo after an initial implantation of the Compound 1 IVT implant. In some instances, the responder has a BCVA of 88 letters and a CST of 381 μm 7 Mo after an initial implantation of the Compound 1 IVT implant. In some instances, such as over the course of several months (e.g., 9 months (M9)) post-implantation of the Compound 1 IVT implants, the responder has a substantial (e.g., 15% or more) decrease in CST (FIG. 2B). In some instances, the responder has a steady decrease in CST over several months (e.g., 9 months (M9) post-implantation of the Compound 1 IVT implants (e.g., about a 20% decrease from baseline CST)—see FIG. 2B), such as, demonstrating the durability of the Compound IVT implant. In some instances, such as from months 1-6 (M1-M6), the visual acuity (e.g., as measured by BCVA) of the responder is maintained (FIG. 2C). In some instances, the responder has a maintained BCVA (e.g., of about 75 letters (FIG. 2C)). In some instances, such as from months 6-8 (M6-M8), the visual acuity (e.g., as measured by BCVA) of the responder increases (e.g., by about 20% from baseline BCVA—see FIG. 2C).

FIG. 2D-2F is representative of a responder (profile), such as a responder profile for a prior treated individual receiving two Compound 1 IVT implants. Panel A-D of FIG. 2D show the edema of the responder at baseline/screening (BCVA is 69 letters, CST is 389 μm), 1 month (M1) after an initial implantation of the Compound 1 IVT implant (BCVA is 73 letters, CST is 250 μm), 4 Mo after an initial implantation of the Compound 1 IVT implant (BCVA is about 75 letters, CST is 242 μm), and 8 Mo after an initial implantation of the Compound 1 IVT implant (BCVA is 76 letters, CST is 241 μm). In some instances, such as over the course of several months (e.g., 8 months (M8)) post-implantation of the Compound 1 IVT implants, the responder has a substantial (e.g., 35% or more) decrease in CST (FIG. 2E). In some instances, the responder has a maintained CST over several months (e.g., 8 months (M8) post-implantation of the Compound 1 IVT implants (e.g., about a 35% decrease from baseline CST)—see FIG. 2BE), such as, demonstrating the durability of the Compound IVT implant. In some instances, such as from months 1-6 (M1-M6), the visual acuity (e.g., as measured by BCVA) of the responder is maintained (FIG. 2F). In some instances, the responder has a maintained BCVA (e.g., of about 75 letters (FIG. 2E)).

In some instances, one or more implant described herein, such as a Compound 1 IVT implant, is administered to an individual in need thereof, such as an individual diagnosed with a retinal vein occlusion (RVO). In some instances, the individual in need thereof has received a previous treatment (for RVO) prior to receiving the Compound 1 IVT implant. In some instances, one implant described herein, such as a Compound 1 IVT implant, is administered to an individual in need thereof. In some instances, two implants described herein, such as a Compound 1 IVT implant, are (sequentially, such as, in series or one month or more after receiving the first implant dose) administered to an individual in need thereof. In some instances, a first implant dose is administered and then a second (e.g., retreatment) implant dose is administered, such as one month or more after receiving the first implant dose.

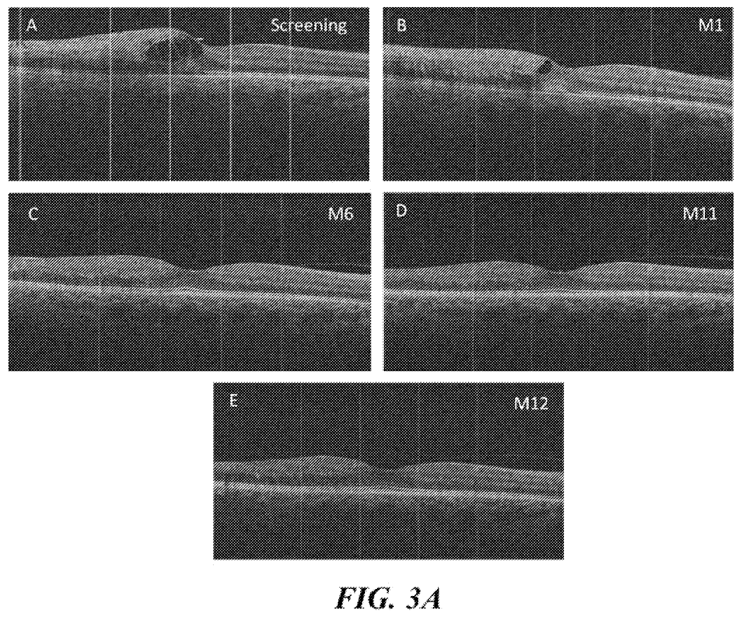

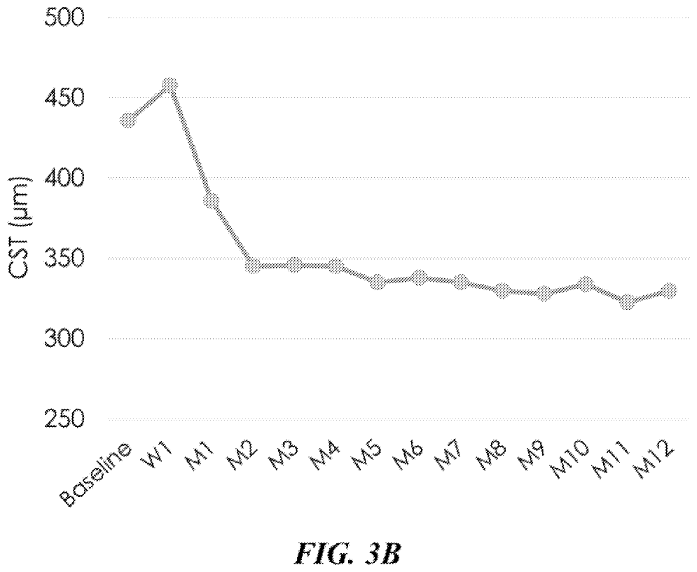

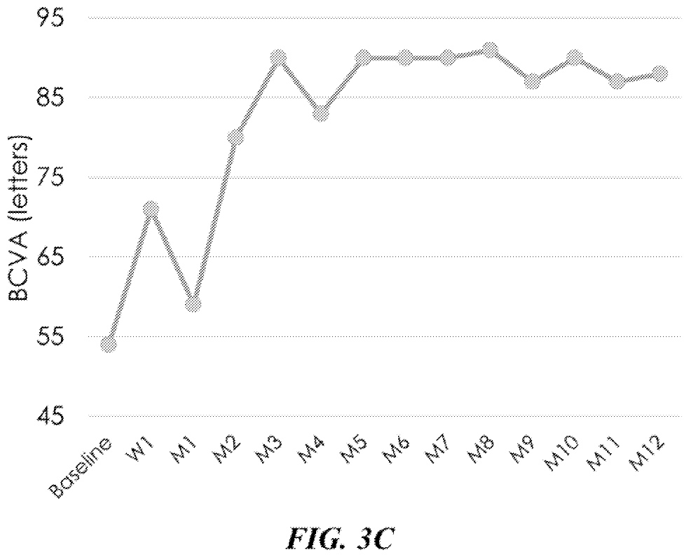

In some instances, a single Compound 1 IVT implant is administered to (e.g., at least 10) individuals diagnosed with RVO (e.g., hemiretinal vein occlusion (HRVO)) and a number of those individuals, such as at least 50% (e.g., 50% or more, 75% or more, or 90% or more) of those individuals, respond (favorably) to the Compound 1 IVT implant. FIG. 3A-3C is representative of a responder (profile), such as a responder profile for a treatment naïve individual receiving the Compound 1 IVT implant. Panel A-E of FIG. 3A show the edema or occlusion of the responder at baseline/screening (BCVA is 62 letters, CST is 436 μm), 1 month (M1) after an initial implantation of the Compound 1 IVT implant (BCVA is 59 letters, CST is 386 μm), 6 Mo after an initial implantation of the Compound 1 IVT implant (BCVA is 90 letters, CST is 338 μm), 11 Mo after an initial implantation of the Compound 1 IVT implant (BCVA is 87 letters, CST is 323 μm), and 12 Mo after an initial implantation of the Compound 1 IVT implant (BCVA is about 85 letters, CST is about 330 μm). In some instances, such as within one-month of implantation of the Compound 1 IVT implant, the responder has a decrease in CST (−50 μm (about a 10% decrease from baseline CST); 438 μm to 386 μm—see FIG. 3B). In some instances, such as out to month 2 (M2), the responder has a strong decline in CST, such as, where CST reaches 345 μm (about a 30% decrease from baseline CST) by M2 (FIG. 3A and FIG. 3B). In some instances, such as at month 3 (M3), the responder has (rapid) letter gain, such as gaining 28 letters (about a 40% increase from baseline BCVA) with best-achieved BCVA being 90 letters (FIG. 3C). In some instances, such as out to month 11 (M11), the individual continues to respond well to Compound 1 IVT implant (e.g., demonstrating the durability of a single Compound 1 IVT implant), such as having a decrease in CST to lowest achieved thickness (e.g., of 323 μm (about a 30% decrease from baseline CST)) and/or vision improvement (e.g., to 87 letters (about a 40% increase from baseline BCVA))—see FIG. 3B and FIG. 3C.

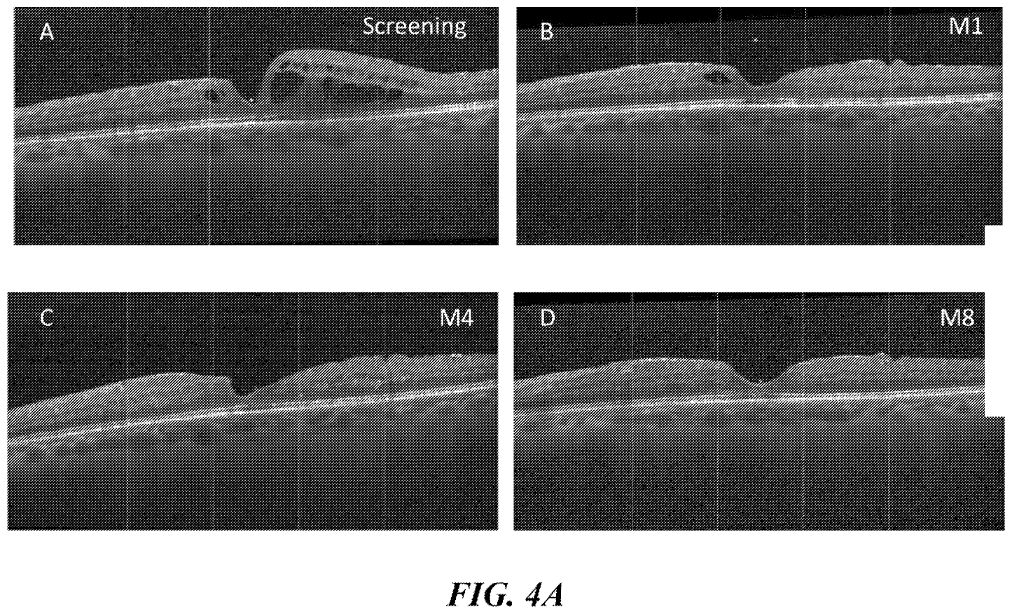

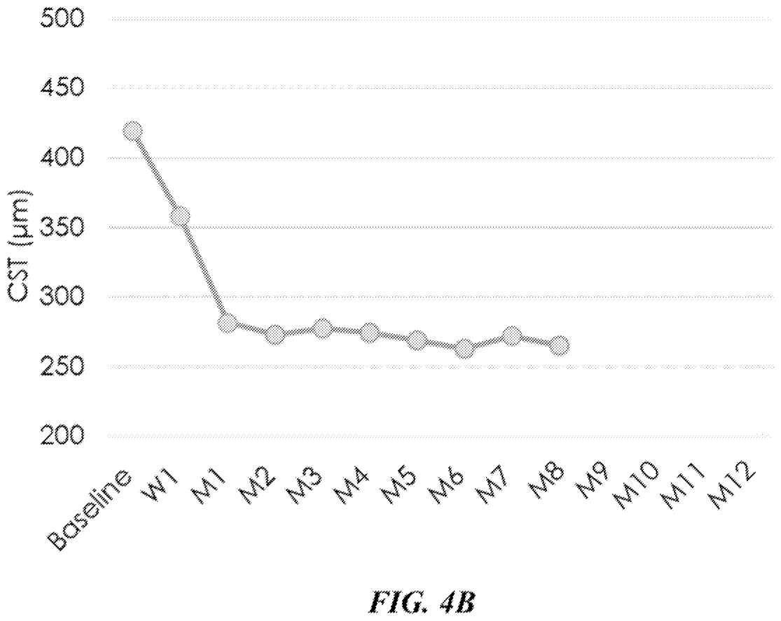

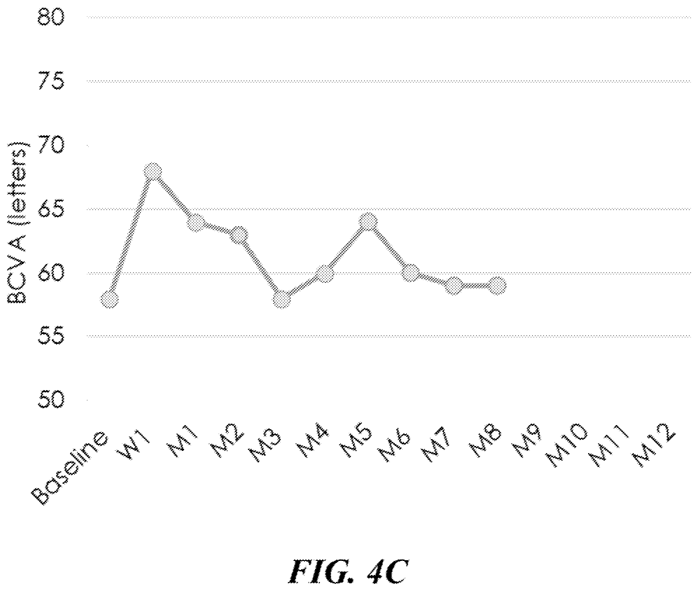

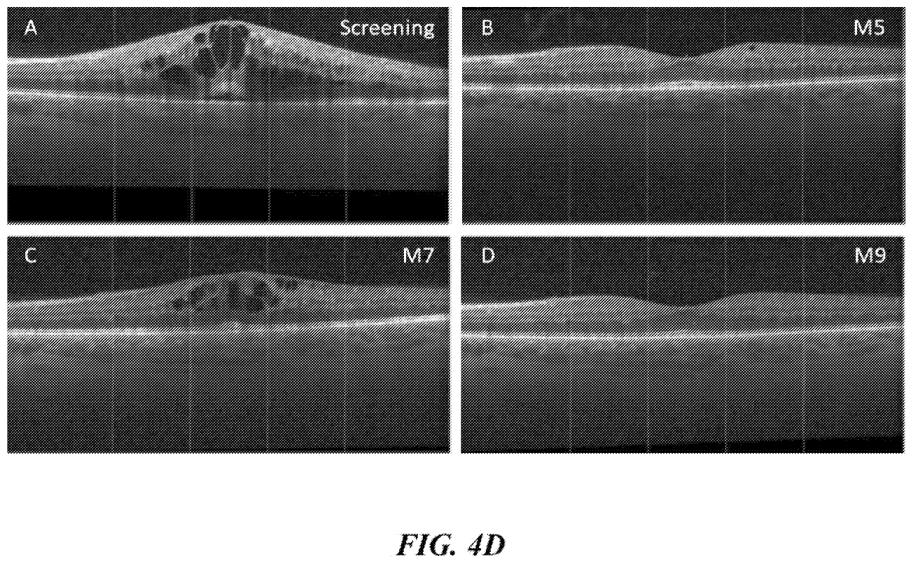

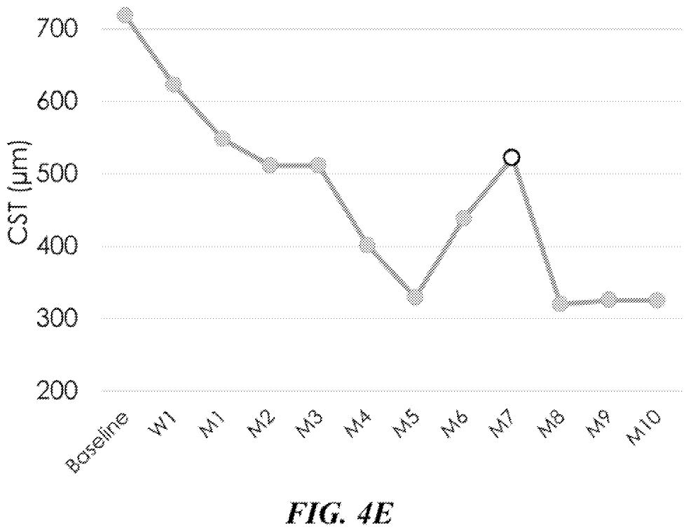

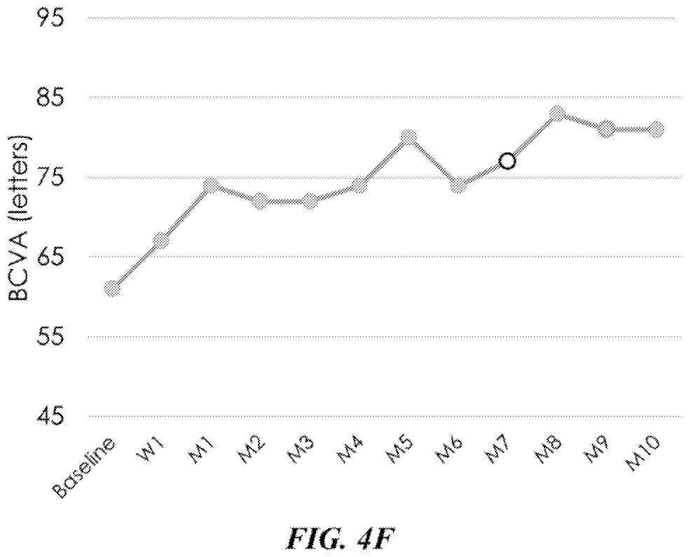

In some instances, two Compound 1 IVT implants are administered to (e.g., at least 5) individuals diagnosed with RVO and a number of those individuals, such as at least 50% (e.g., 50% or more, 75% or more, or 90% or more) of those individuals, respond (favorably) to the Compound 1 IVT implants (e.g., FIGS. 4A-4F).

FIG. 4A-4C is representative of a responder (profile), such as a responder profile for an individual that has received a previous treatment for RVO (e.g., anti-VEGF therapeutics) prior to receiving the Compound 1 IVT implants (e.g., two Compound 1 IVT implants). Panel A-D of FIG. 4A show the occlusion or edema of the responder at baseline/screening (BCVA is 65 letters, CST is 419 μm), 1 month (M1) after an initial implantation of the Compound 1 IVT implant (BCVA is 64 letters, CST is 282 μm), 4 Mo after an initial implantation of the Compound 1 IVT implant (BCVA is 60 letters, CST is 274 μm), and 8 months (M8) after an initial implantation of the Compound 1 IVT implant (BCVA is 59 letters, CST is 265 μm). In some instances, such as within one-month of implantation of the Compound 1 IVT implants, the responder has a (strong and rapid) decrease in CST (−137 μm (about a 30% decrease from baseline CST); 419 μm to 282 μm—see FIG. 4A and FIG. 4B). In some instances, the decrease in CST in the individual is maintained over several months (e.g., 8 months (M8) post-implantation of the Compound 1 IVT implants—see FIG. 4B), such as, demonstrating the durability of the Compound 1 IVT implants. In some instances, such as from months 1-2 (M1-M2), the visual acuity (e.g., as measured by BCVA) of the responder increases, such as by about 20% from baseline BCVA (FIG. 4C). In some instances, the responder has a maintained BCVA (e.g., of about 60 letters (FIG. 4C)).

FIG. 4D-4F is representative of a responder (profile), such as a responder profile for a treatment naïve individual receiving the Compound 1 IVT implants. Panel A-D of FIG. 4A show the edema or occlusion of the responder at baseline/screening (BCVA is 59 letters, CST is 718 μm), 5 months (M5) after an initial implantation of the Compound 1 IVT implant (BCVA is 80 letters, CST is 329 μm), 7 Mo after an initial implantation of the Compound 1 IVT implant (BCVA is 77 letters, CST is 522 μm), and 9 Mo after an initial implantation of the Compound 1 IVT implant (BCVA is 81 letters, CST is 325 μm). In some instances, such as within one-month of implantation of the Compound 1 IVT implants, the responder has a (strong and rapid) decrease in CST (about 200 μm (about a 25% decrease from baseline CST); about 710 μm to 548 μm—see FIG. 4D and FIG. 4E). In some instances, the decrease in CST in the individual is maintained over several months (e.g., 4 months (M4) post-implantation of the Compound 1 IVT implants—see FIG. 4E), such as, demonstrating the durability of the Compound 1 IVT implants. In some instances, such as from months 1-4 (M1-M5), the visual acuity (e.g., as measured by BCVA) of the responder increases, such as by about 20% from baseline BCVA (FIG. 4F). In some instances, the responder receives a retreatment dose (open circle of FIG. 4E and FIG. 4F), such as two Compound 1 IVT implants. In some instances, such as after retreatment, the responder continues to respond well (e.g., out to month 10 (M10)) to Compound 1 IVT implant, such as having a decrease in CST (e.g., to 325 μm (about a 50% decrease from baseline CST)) and/or vision improvement (e.g., to about 80 letters) (see FIGS. 4D-4F).

FIGS. 5A-B show the mean change in central subfield thickness (CST) and mean change in BVCA, respectively, for Group 1 (grey) and Group 2 (black) responders with RVO. The initial number of individuals receiving Compound 1 intravitreal (IVT) implant in RVO Group 1 at baseline is 10. The initial number of individuals receiving Compound 1 IVT implant in RVO Group 2 at baseline is 15. In some instances, the data is an average aggregate for the Group 1 (1 implant of Compound 1 (containing 92 μg+/−15% of Compound 1)) and Group 2 (2 implants of Compound 1 (each implant containing 92 μg+/−15% of Compound 1)) RVO cohorts. In some instances, the mean change in CST is presented relative to the CST recorded at the baseline visit prior to treatment administration. In some instances, the mean change in BCVA is presented relative to the BCVA recorded at the baseline visit prior to treatment administration. In some instances, the data presented in FIGS. 5A-B includes individuals up to time of rescue, such as, where the number of individuals at each timepoint may decrease if an individual receives rescue therapy before or at a respective timepoint the study. In some instances, both Group 1 and Group 2 cohorts show a rapid response to the Compound 1 IVT implant therapy. In some instances, both Group 1 and Group 2 cohorts have an average CST decrease of at least about 50 μm (e.g., about 100 μm or more), such as, in the first week, such as, indicating a strong, fast-onset biological effect. In some instances, such as out to month 2 (M2) (e.g., RVO Group 1) or month 3 (M3) (e.g., RVO Group 2), CST continues to decrease (e.g., for a prolonged period of time, such as several months). In some instance, such as at month 2 (M2) (e.g., RVO Group 1) or month 3 (M3) (e.g., RVO Group 2), the CST reaches a plateau. In some instance s, such as out to month 11 (M11) (e.g., RVO Group 1) or month 7 (M7) (e.g., RVO Group 2), the plateau is maintained (e.g., for a prolonged period of time, such as several months). In some instances, both Group 1 and Group 2 cohorts have an average BCVA increase of at least about 3 letters or more (e.g., about 4 letters or more, about 5 letters or more), such as, in the first week of treatment, indicating a strong and rapid onset of vision improvement after an individual receives an implant described herein. In some instances, such as out to month 3 (M3), mean change in BCVA continues to increase (e.g., for an extended period of time, such as for several months).

FIG. 5C shows the mean change in CST for Group 1 (grey) and Group 2 (black) responders with RVO as well as for individuals with RVO who received Implant A (Group 3, dotted line). Implant A is an implant that comprises dexamethasone in a polymer matrix (e.g., such as described in Haller et al.; Randomized, sham-controlled trial of dexamethasone intravitreal implant in patients with macular edema due to retinal vein occlusion, Ophthalmology. 2010 June; 117(6)). In some instances, the data is an average aggregate for the Group 1 (1 implant of Compound 1 (containing 92 μg+/−15% of Compound 1)), Group 2 (2 implants of Compound 1 (each implant containing 92 μg+/−15% of Compound 1)) RVO cohorts, and Group 3 (Implant A). In some instances, CST continues to decrease in Groups 1 and 2 for an extended period of time, such as for more than 3 months longer than the CST of the individuals in Group 3). In some instances, a decreased CST relative to baseline is maintained and/or decreases for a longer period of time in Groups 1 and 2 compared to Group 3 (e.g., see M3 vs M6 of FIG. 5C).

FIG. 5D shows the mean change in BCVA for Group 1 (grey) and Group 2 (black) responders with RVO as well as for individuals with RVO who received Implant A (Group 3, dotted line). Implant A is described elsewhere herein. In some instances, the data is an average aggregate for the Group 1 (1 implant of Compound 1 (containing 92 μg+/−15% of Compound 1)), Group 2 (2 implants of Compound 1 (each implant containing 92 μg+/−15% of Compound 1)) RVO cohorts, and Group 3 (Implant A). In some instances, vision improvement (e.g., as measured by BCVA) continues to increase in Groups 1 and 2 for an extended period of time, such as for more than 4 months longer than the vision improves in the individuals in Group 3). In some instances, increased BCVA relative to baseline is maintained and/or increases for a longer period of time in Groups 1 and 2 compared to Group 3 (e.g., see M2 vs M6 of FIG. 5D).

In some instances, the results described (e.g., see FIGS. 5C and 5D) herein demonstrate that an implant described herein (e.g., a Compound 1 IVT implant) has as good or better efficacy than a clinically approved IVT implant (e.g., Implant A, Anti-VEGF A, Anti-VEGF B, or Anti-VEGF C). In some instances, the results described herein demonstrate that an implant described herein (e.g., a Compound 1 IVT implant) has as good or better efficacy over a longer period of time when compared to a clinically approved IVT implant (e.g., Implant A). In some instances, the results described herein demonstrate that an implant described herein (e.g., a Compound 1 IVT implant) has as good or better efficacy for at least three more months than a clinically approved IVT implant (e.g., Implant A).

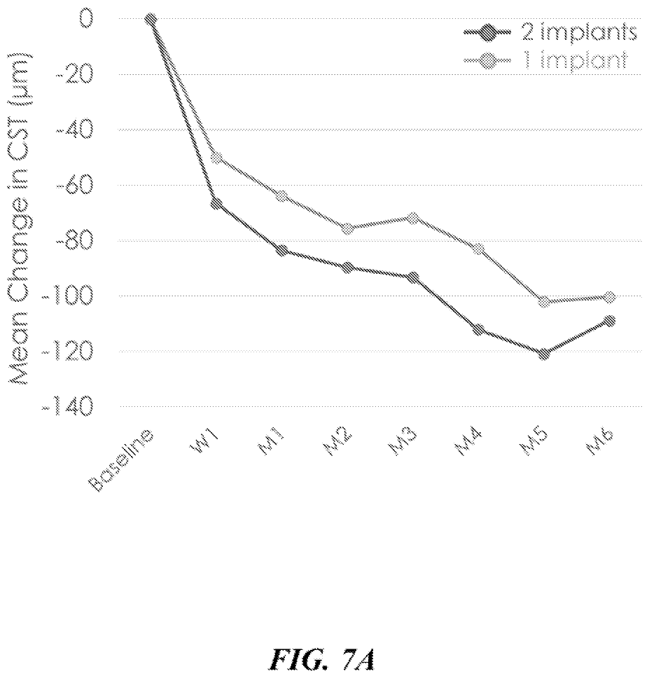

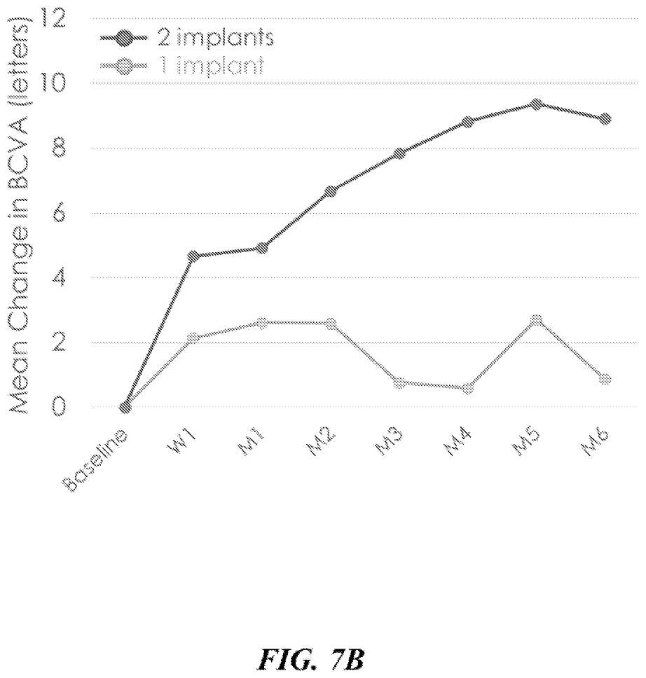

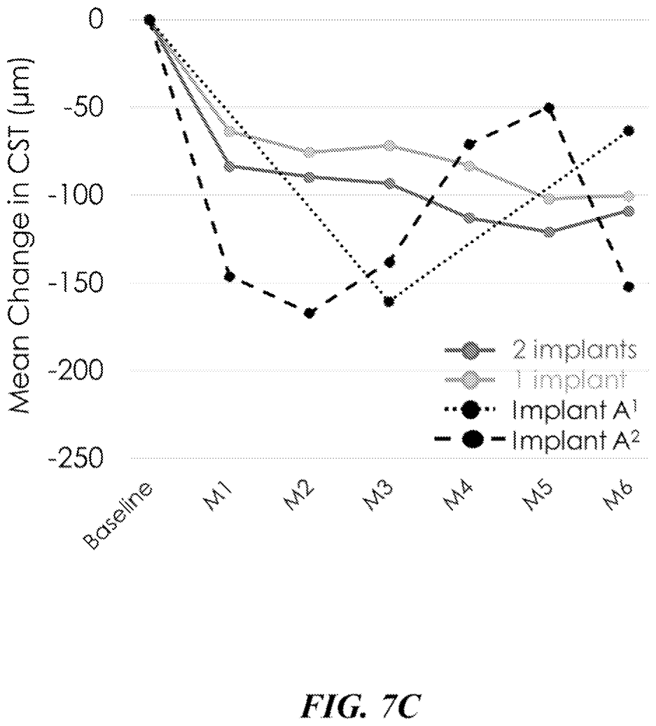

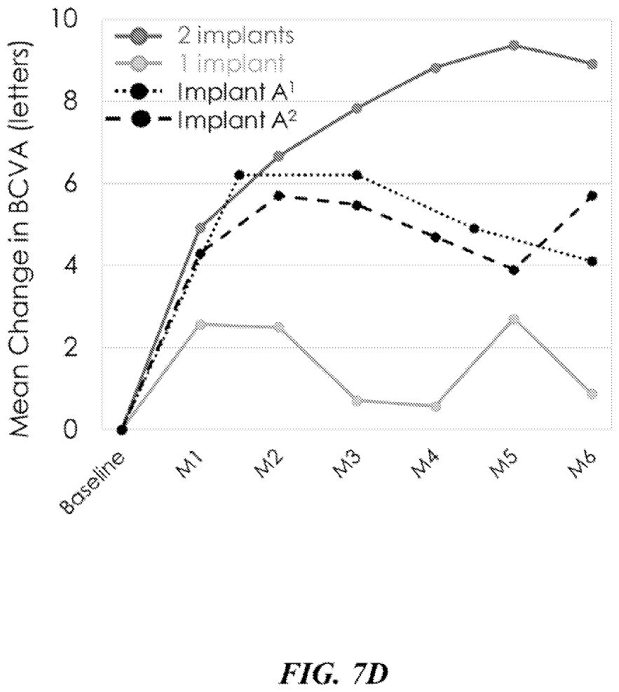

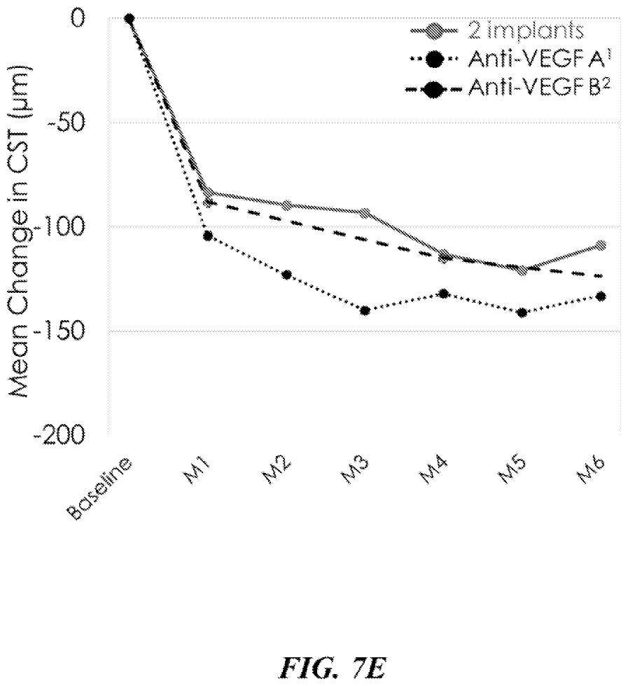

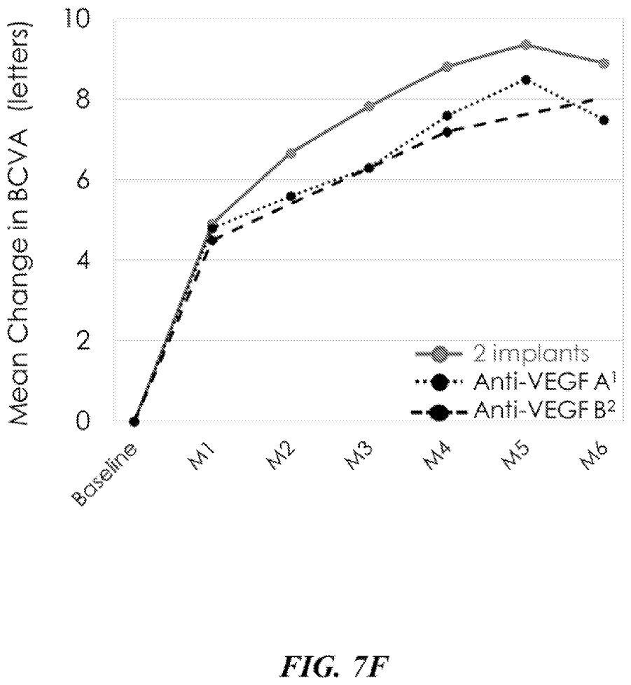

FIGS. 7A-B show the mean change in central subfield thickness (CST) and mean change in BVCA, respectively, for Group 1 (grey) and Group 2 (black) responders with DME. The initial number of individuals receiving Compound 1 intravitreal (IVT) implant in DME Group 1 at baseline is 23. The initial number of individuals receiving Compound 1 IVT implant in DME Group 2 at baseline is 12. In some instances, the data is an average aggregate for the Group 1 (1 implant of Compound 1 (containing 92 μg+/−15% of Compound 1)) and Group 2 (2 implants of Compound 1 (each implant containing 92 μg+/−15% of Compound 1)) DME cohorts. In some instances, the mean change in CST is presented relative to the CST recorded at the baseline visit prior to treatment administration. In some instances, the mean change in BCVA is presented relative to the BCVA recorded at the baseline visit prior to treatment administration. In some instances, the data presented in FIGS. 7A-B includes individuals up to time of rescue, such as, where the number of individuals at each timepoint may decrease if an individual receives rescue therapy before or at a respective timepoint in the study. In some instances, both Group 1 and Group 2 cohorts show a rapid response to the Compound 1 IVT implant therapy. In some instances, both Group 1 and Group 2 cohorts have an average CST decrease of at least about 50 μm (e.g., about 100 μm or more), such as, in the first week, such as, indicating a strong, fast-onset biological effect. In some instances, such as out to month 2 (M2) (e.g., DME Group 1) or month 5 (M3) (e.g., DMIE Group 2), CST continues to decrease (e.g., for a prolonged period of time, such as several months). In some instance, the CST continues to decrease during the course of treatment. In some instances, such as out to month 6 (M6) (e.g., DME Groups 1 and 2), a decrease in CST is maintained. In some instances, both Group 1 and Group 2 cohorts have an average BCVA increase of at least about 3 letters or more. In some instances, Group 2 has an average BCVA increase of about 4 letters or more, about 5 letters or more, about 6 letters or more, about 7 letters or more, about 8 letters or more, such as, over the course of treatment, indicating a strong and rapid onset as well as a maintenance of vision improvement after an individual receives an implant described herein. In some instances, such as out to month 5 (M5), mean change in BCVA continues to increase (e.g., for an extended period of time, such as for several months).

FIG. 7C shows the mean change in CST for Group 1 (grey) and Group 2 (black) responders with DME as well as for individuals with DME who received Implant A (Group 4 (dotted line) and Group 5 (dashed line)). Implant A¹ and Implant A² are each Implant A (as described elsewhere herein) and represent results measured in studies described in Boyer et al. (Ophthalmology. 2014; 121: 1904-1914) and Callanan et al. (Graefes Arch. Clin. Exp. Ophthalmol. 2017; 255:463-473), respectively. In some instances, the data is an average aggregate for the Group 1 (1 implant of Compound 1 (containing 92 μg+/−15% of Compound 1)) and Group 2 (2 implants of Compound 1 (each implant containing 92 μg+/−15% of Compound 1)) DME cohorts as well as Groups 4 (Implant A¹) and 5 (Implant A²). In some instances, CST continues to decrease in Groups 1 and 2 for an extended period of time, such as for more than 3 months longer than the CST of the individuals in Groups 4 and 5). In some instances, a decreased CST relative to baseline is maintained and/or decreases for a longer period of time in Groups 1 and 2 compared to Groups 4 and 5 (e.g., see M2 and M3 vs M6 of FIG. 7C). In some instances, such as described in Callanan et al., Implant A is administered (again) at M5, such as providing a sharp decline in CST from month 5 (M5) to month 6 (M6) of treatment. In some instances, such as described in Boyer et al. and Callanan et al., CST begins increasing substantially (e.g., CST increases by about 100 μm or more) at about month 2 (M2) or month 3 (M3) in individuals administered Implant A. In some instances, CST begins continues decreasing at month 2 (M2), month 3 (M3), month 4 (M4), and month 5 (M5) in individuals administered a Compound 1 IVT implant.

FIG. 7D shows the mean change in BCVA for Group 1 (grey) and Group 2 (black) responders with DME as well as for individuals with MDE who received Implant A (Group 4 (dotted line) and Group 5 (dashed line)). Implant A¹ and Implant A² are described elsewhere herein. In some instances, the data is an average aggregate for the Group 1 (1 implant of Compound 1 (containing 92 μg+/−15% of Compound 1)) and Group 2 (2 implants of Compound 1 (each implant containing 92 μg+/−15% of Compound 1)) DME cohorts as well as Groups 4 (Implant A¹) and 5 (Implant A²). In some instances, vision improvement (e.g., as measured by BCVA) continues to increase in Group 2 for an extended period of time, such as for more than 3 months longer than the vision improves in the individuals in Groups 4 and 5). In some instances, increased BCVA relative to baseline is maintained and/or increases for a longer period of time in Group 2 compared to Groups 4 and 5 (e.g., see M2 vs M6 of FIG. 7D).

FIG. 7E shows the mean change in CST for Group 2 (black) responders with DME as well as for individuals with DME who received Anti-VEGF A (Group 6 (dotted line)) or Anti-VEGF B (Group 6 (dashed line)). Anti-VEGF A is a ranibizumab injection and Anti-VEGF B is a aflibercept injection. Anti-VEGF A¹ represents results measured in studies described in Callanan et al. (Graefes Arch. Clin. Exp. Ophthalmol. 2017; 255:463-473). Anti-VEGF B² represents results measured in studies described in Korobelnik et al. (Ophthalmology 2014; 121:2247-2254). In some instances, the data is an average aggregate for the Group 2 (2 implants of Compound 1 (each implant containing 92 μg+/−15% of Compound 1)) DME cohorts as well as Groups 5 (Anti-VEGF A¹) and 6 (Anti-VEGF B²). In some instances, Anti-VEGF A and Anti-VEGF B are the standard clinically approved implants for treating DME and/or reducing CST levels in individuals with DME. In some instances, Anti-VEGF A and Anti-VEGF B are injected into an eye of an individual in need thereof at least once monthly for the entire treatment period, such chronic and frequent implantation putting a significant burden on the individual. In some instances, an implant described herein, such as a Compound 1 IVT implant, is administered to an individual in need thereof substantially less frequently (e.g., only once or twice yearly) than Anti-VEGF A or Anti-VEGF B, providing a less burden some treatment option for the individual. In some instances, CST continues to decrease in Groups 2, 5, and 6 for an extended period of time, such as for 6 months or more. In some instances, a decreased CST relative to baseline is maintained and/or further decreases for Groups 2, 5, and 6. In some instances, an implant described herein, such as a Compound 1 IVT implant, has as good or better efficacy for at least as long as a clinically approved IVT implant (e.g., Anti-VEGF A and Anti-VEGF B) without requiring monthly implantation, such as providing a treatment option that puts less burden on the individual.

FIG. 7F shows the mean change in BVCA for Group 2 (black) responders with DME as well as for individuals with DME who received Anti-VEGF A (Group 6 (dotted line)) or Anti-VEGF B (Group 6 (dashed line)). Anti-VEGF A and Anti-VEGF B are described elsewhere herein. Anti-VEGF A¹ represents results measured in studies described in Callanan et al. (Graefes Arch. Clin. Exp. Ophthalmol. 2017; 255:463-473). Anti-VEGF B² represents results measured in studies described in Korobelnik et al. (Ophthalmology 2014; 121:2247-2254). In some instances, the data is an average aggregate for the Group 2 (2 implants of Compound 1 (each implant containing 92 μg+/−15% of Compound 1)) DME cohorts as well as Groups 5 (Anti-VEGF A¹) and 6 (Anti-VEGF B²). In some instances, Anti-VEGF A and Anti-VEGF B are the standard clinically approved implants for treating DME and/or improving vision in individuals with DME. In some instances, Anti-VEGF A and Anti-VEGF B are injected into an eye of an individual in need thereof at least once monthly for the entire treatment period, such chronic and frequent injection putting a significant burden on the individual. In some instances, an implant described herein, such as a Compound 1 IVT implant, is administered to an individual in need thereof substantially less frequently (e.g., only once or twice yearly) than Anti-VEGF A or Anti-VEGF B, providing a less burdensome treatment option for the individual. In some instances, BCVA continues to increase in Groups 2, 5, and 6 for an extended period of time, such as for 5 months or more. In some instances, an increased BCVA relative to baseline is maintained and/or increases for Groups 2, 5, and 6. In some instances, an implant described herein, such as a Compound 1 IVT implant, has as good or better efficacy for at least as long as a clinically approved IVT injection (e.g., Anti-VEGF A and Anti-VEGF B) without requiring monthly implantation, such as providing a treatment option that puts less burden on the individual.

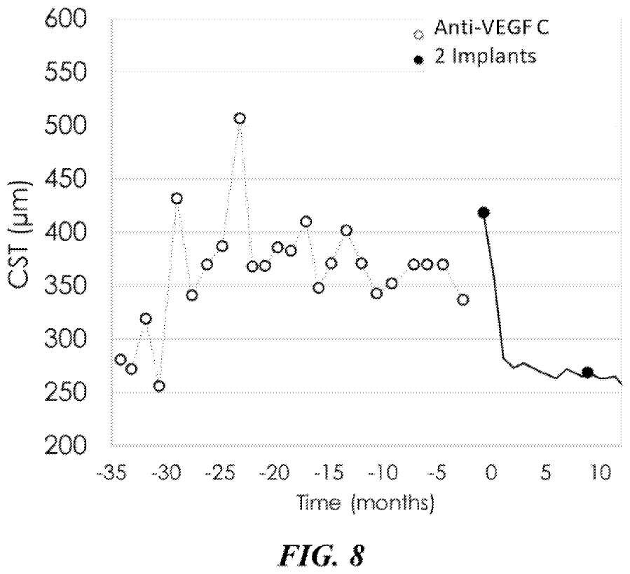

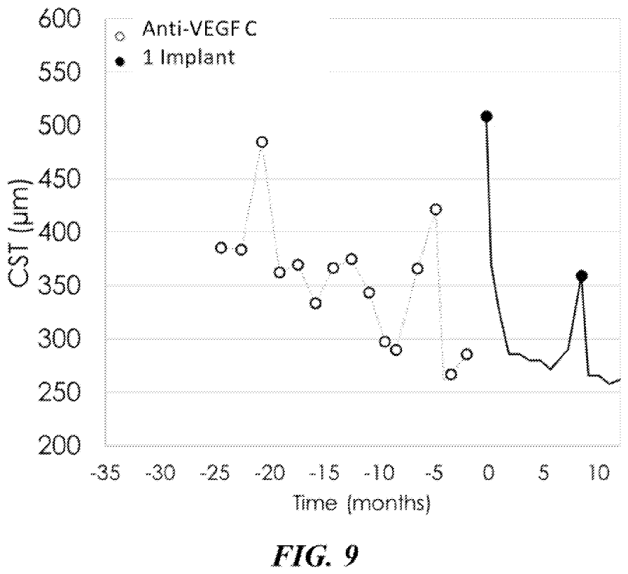

In FIGS. 8 and 9, Anti-VEGF C is an aflibercept injection and the results for Anti-VEGF C shown in FIGS. 8 and 9 are repurposed from studies described in NCT04576689. In some instances, FIGS. 8 and 9 further demonstrate the robustness of an implant described herein, such as a Compound 1 IVT implant, e.g., as compared to clinically approved IVT injections (e.g., Anti-VEGF C). For example, each circle (i.e., closed or open) shown in FIGS. 8 and 9 represents an injection event of the implant, illustrating how much more frequently (e.g., once monthly) Anti-VEGF C is administered compared to an implant described herein, such as a Compound 1 IVT implant (e.g., once every 6 months). Additionally, FIGS. 8 and 9 demonstrate that even when one (FIG. 8) or two (FIG. 9) implant described herein, such as a Compound 1 IVT implant, is implanted into an eye of an individual once every 6 months, the CST unexpectedly decreases more or to similar levels compared to the CST decrease measured for once-monthly Anti-VEGF C injection. Furthermore, FIGS. 8 and 9 demonstrate that an individual having an implant described herein administered after (e.g., after a washout period, such as one or more week after) an Anti-VEGF injection described herein can unexpectedly have an immediate and robust improvement (decrease) in CST levels. In some instances, FIGS. 8 and 9 demonstrate that administering one or more implant described herein to an eye of an individual extends treatment of an anti-VEGF therapy (e.g., injection) described herein, such as by about one month or more, six months or more, or twelve months or more (e.g., with only one or two implantation events being required for the one or more implant).

In some instances, Table 1 summarizes a comparison in CST at baseline, 3 months (M3), and 6 months (M6) for (RVO) Group 1, Group 2, and Implant A—a clinically-approved dexamethasone intravitreal implant. In some instances, such as 3 months after implantation of Compound 1 IVT implant, the mean change in CST in Group 2 improves more than the mean change in CST of Implant A, such as 3 months after implantation of Implant A. In some instances, such as 6 months after implantation of Compound 1 IVT implant, the mean change in CST in Group 1 and Group 2 improves more than the mean change in CST of Implant A, such as 6 months after implantation of Implant A. In some instances, such as 6 months after implantation of Compound 1 IVT implant, the mean change in CST in Group 1 and Group 2 improves more than the mean change in CST of Implant A, such as 6 months after implantation of Implant A.

In some instances, Table 1 shows that one or more Compound 1 IVT implant provides responders with a bigger improvement in CST than Implant A. In some instances, Table 1 shows that one or more Compound 1 IVT implant provides responders with a more robust improvement in CST than Implant A. In some instances, Table 1 shows that one or more Compound 1 IVT implant provides responders with a more durable improvement in CST than Implant A.

TABLE 1

| | | | | | Implant A | |
|---|---|---|---|---|---|---|
| | Group 1 | | Group 2 | | (0.7 mg) | |
| Baseline CST | 585 | μm | 563 | μm | 562 | μm |
| Mean Change in | −172 | μm | −193 | μm | −208 | μm |
| CST at M3 | | | | | | |
| Mean Change in | −260 | μm | −193 | μm | −119 | μm |
| CST at M6 | | | | | | |

In some instances, Table 2 summarizes a comparison in BVCA at baseline, 3 months (3M), and 6 months (M6) for (RVO) Group 1, Group 2, and Implant A—a clinically-approved dexamethasone intravitreal implant. In some instances, such as 3 months after implantation of Compound 1 IVT implant, the mean change in BCVA in Group 1 and Group 2 improves more than the mean change in BCVA of Implant A, such as 3 months after implantation of Implant A. In some instances, such as 6 months after implantation of Compound 1 IVT implant, the mean change in BCVA in Group 1 improves more than the mean change in BCVA of Implant A, such as 6 months after implantation of Implant A. In some instances, such as 3 months after implantation of Compound 1 IVT implant, the mean change in BCVA in Group 1 and Group 2 improves more than the mean change in BCVA of Implant A, such as 3 months after implantation of Implant A. In some instances, such as 6 months after implantation of Compound 1 IVT implant, the mean change in BCVA in Group 1 and Group 2 improves more than the mean change in BCVA of Implant A, such as 6 months after implantation of Implant A.

In some instances, Table 2 shows that one or more Compound 1 IVT implant provides responders with a higher improvement in BCVA than Implant A. In some instances, Table 2 shows that one or more Compound 1 IVT implant provides responders with a more robust improvement in BCVA than Implant A. In some instances, Table 2 shows that one or more Compound 1 IVT implant provides responders with a more durable improvement in BCVA than Implant A.

TABLE 2

| | | | | | Implant A | |
|---|---|---|---|---|---|---|
| | Group 1 | | Group 2 | | (0.7 mg) | |
| Baseline BCVA | 62 | letters | 59 | letters | 54.3 | letters |
| Mean Change in | +9 | letters | +8 | letters | 7.2 | letters |
| BCVA at M3 | | | | | | |
| Mean Change in | +11 | letters | +7 | letters | 5 | letters |
| BCVA at M6 | | | | | | |

In some instances, Table 3 summarizes a comparison in CST at baseline, 3 months (M3), and 6 months (M6) for (DME) Group 1, Group 2, Implant A (a clinically-approved dexamethasone intravitreal implant), and Anti-VEGF A (a clinically approved anti-VEGF intravitreal injection). In some instances, such as 6 months after implantation of Compound 1 IVT implant, the mean change in CST in Group 1 and Group 2 improves more than the mean change in CST of Implant A, such as 6 months after implantation of Implant A. In some instances, such as 6 months after implantation of Compound 1 IVT implant, the improvement in CST in Group 1 and Group 2 is comparable the mean change in CST of Anti-VEGF A, such as 6 months after injection of Anti-VEGF A. In some instances, an implant described herein (e.g., Compound 1 IVT implant) is injected into individuals of Group 1 and Group 2 substantially less frequently (e.g., twice yearly) than an Anti-VEGF injection is injected into the individuals of the Anti-VEGF A study described herein (e.g., once monthly).

In some instances, Table 3 shows that one or more Compound 1 IVT implant provides responders with a bigger improvement in CST than Implant A. In some instances, Table 3 shows that one or more Compound 1 IVT implant provides responders with a more robust improvement in CST than Implant A. In some instances, Table 3 shows that one or more Compound 1 IVT implant provides responders with a more durable improvement in CST than Implant A. In some instances, Table 3 shows that one or more Compound 1 IVT implant is injected much less frequently than Anti-VEGF A injections and one or more Compound 1 IVT implant provides responders with a similar improvement in CST compared to Anti-VEGF A injections.

TABLE 3

| | Group 1 | | Group 2 | | Implant A¹ | | Implant A² | | Anti-VEGF A | |
|---|---|---|---|---|---|---|---|---|---|---|
| Baseline CST | 495 | μm | 506 | μm | 463 | μm | 465 | μm | 471 | μm |
| Mean Change in | −72 | μm | −93 | μm | −160 | μm | −138 | μm | −140 | μm |
| CST at M3 | | | | | | | | | | |
| Mean Change in | −100 | μm | −109 | μm | −63 | μm | −152 | μm | −133 | μm |
| CST at M6 | | | | | | | | | | |

In some instances, Table 4 summarizes a comparison in BVCA at baseline, 3 months (3M), and 6 months (M6) for (RVO) Group 1, Group 2, Implant A (a clinically-approved dexamethasone intravitreal implant), and Anti-VEGF A (a clinically approved anti-VEGF intravitreal injection). In some instances, such as 3 months after implantation of Compound 1 IVT implant, the mean change in BCVA in Group 2 improves more than the mean change in BCVA of Implant A, such as 3 months after implantation of Implant A. In some instances, such as 6 months after implantation of Compound 1 IVT implant, the mean change in BCVA in Group 2 improves more than the mean change in BCVA of Implant A, such as 6 months after implantation of Implant A. In some instances, such as 3 and months after implantation of Compound 1 IVT implant, the improvement in visual acuity in Group 2 is comparable the improvement in visual acuity of Anti-VEGF A, such as 3 and 6 months after implantation of Anti-VEGF A. In some instances, an implant described herein (e.g., Compound 1 IVT implant) is injected into individuals of Group 1 and Group 2 substantially less frequently (e.g., twice yearly) than an Anti-VEGF injection is injected into the individuals of the Anti-VEGF A study described herein (e.g., once monthly).

In some instances, Table 4 shows that one or more Compound 1 IVT implant provides responders with a higher improvement in BCVA than Implant A. In some instances, Table 4 shows that one or more Compound 1 IVT implant provides responders with a more robust improvement in BCVA than Implant A. In some instances, Table 4 shows that one or more Compound 1 IVT implant provides responders with a more durable improvement in BCVA than Implant A. In some instances, Table 4 shows that one or more Compound 1 IVT implant is injected much less frequently than Anti-VEGF A injections and one or more Compound 1 IVT implant provides responders with a similar improvement in visual acuity compared to Anti-VEGF A injections.

TABLE 4

| | Group 1 | | Group 2 | | Implant A¹ | | Implant A² | | Anti-VEGF A | |
|---|---|---|---|---|---|---|---|---|---|---|
| Baseline BCVA | 67 | letters | 64 | letters | 56 | letters | 60 | letters | 60 | letters |
| Mean Change in | +1 | letters | +8 | letters | +6 | letters | +6 | letters | +6 | letters |
| BCVA at M3 | | | | | | | | | | |
| Mean Change in | +1 | letters | +9 | letters | +4 | letters | +6 | letters | +8 | letters |
| BCVA at M6 | | | | | | | | | | |

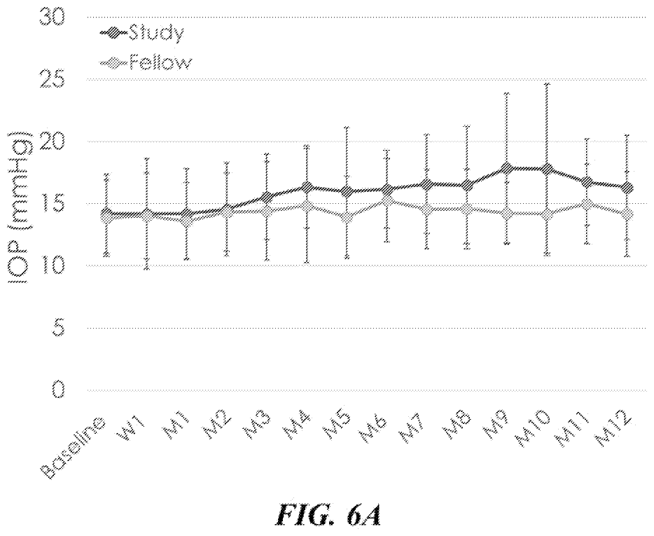

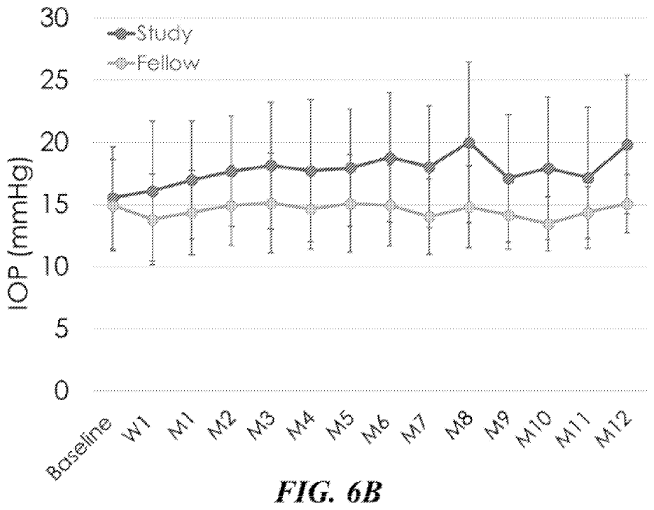

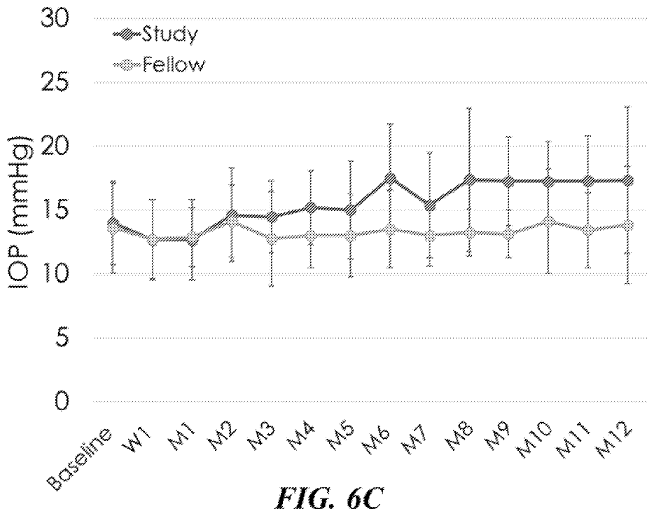

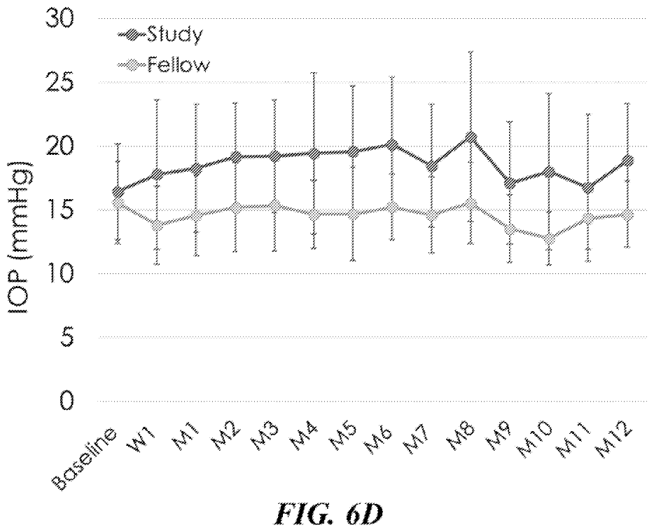

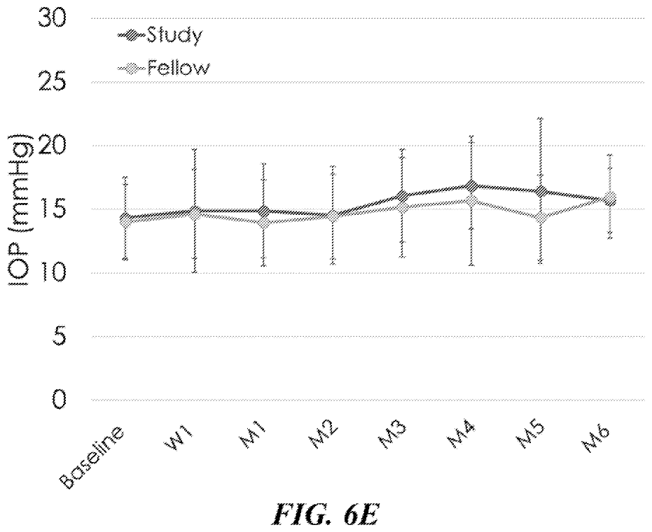

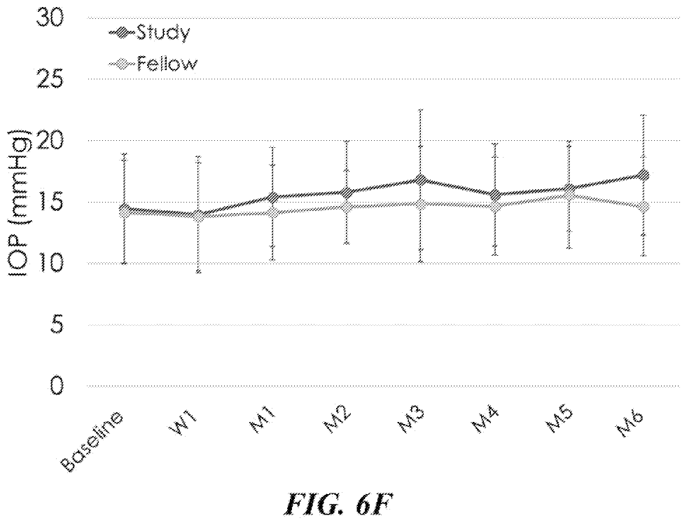

FIGS. 6A-6F show comparison of the change in intraocular pressure (IOP) levels in an eye of an individual (or a population of individuals) receiving one or more implant described herein (e.g., one or more Compound 1 IVT implant) (black) versus fellow eye (grey). In some instances, the individual receiving one or more implant is in Group 1 (FIGS. 6A, 6C, 6E) or Group 2 (FIGS. 6B, 6D, 6F). In some instances, IOP data is presented as aggregate data including RVO and DME patients as well as those on IOP-lowering medication in their respective Group (FIGS. 6A and 6B). In some instances, IOP data is presented as aggregate data of RVO patients (FIGS. 6C, 6D). In some instances, IOP data is presented as aggregate data of DME patients (FIGS. 6E, 6F). In some instances, the IOP measurements taken from the study eye and the fellow eye in the Group 1 cohort are similar at all timepoints out to Month 12 (e.g., FIGS. 6A, 6E, 6F). In some instances, there is no steroid-related treatment response in the Group 1 cohort. In some instances, such as in the Group 2 cohort, the IOP of eyes receiving the Compound 1 IVT implant is slightly higher on average than control eyes (e.g., those not receiving the implant) (e.g., FIG. 6B and FIG. 6D). In some instances, such as by Month 8, there is a statistically significant difference in the average IOP of the study eye (e.g., 23 mmHg) compared to the fellow eye (e.g., 16 mmHg) (FIG. 6B). In some instances, the average IOP of the study eye stabilizes compared to the fellow eye. In some instances, such as shown in FIG. 6A and/or FIG. 6B, a minimum effective dose to elicit a therapeutic benefit (e.g., 70 μg of dexamethasone), such as in the absence of the typical steroid-induced IOP elevation, is provided to an individual in need thereof. In some instances, the IOP measurements taken from the study eye and the fellow eye in the Group 1 cohort are within about 5 mmHg for all timepoints out to Month 12 (FIG. 6C). In some instances, the IOP measurements taken from the study eye and the fellow eye in the Group 1 cohort are similar all timepoints out to Month 6 (FIG. 6E). In some instances, the IOP measurements taken from the study eye and the fellow eye in the Group 2 cohort are similar at all timepoints out to Month 6 (FIG. 6F).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Examples

Evaluation of Safety and Efficacy of Compound 1 Intravitreal (IVT) Implant—A Sustained Dose Dexamethasone Therapy for the Treatment of Patients with Diabetic Macular Edema (DME) and Macular Edema due to Retinal Vein Occlusion (RVO)

The study described herein is a Phase II, multi-center, single-masked (assessors) dose-ranging study designed to evaluate the comparative safety and preliminary efficacy of 2 dosage regimens of the Compound 1 IVT Dexamethasone Implant in patients with DME and RVO.

Up to 60 individuals (up to 60 eyes) with DME or RVO across 2 treatment groups (e.g., Group 1 and Group 2) are enrolled in the study. Individuals are identified and recruited through the clinic population of ophthalmology centers specializing in the diagnosis and treatment of retinal diseases. All individuals are followed for a planned evaluation period of 18 months.

### Group 1

The Group 1 treatment group consists of at least 10 and up to about 33 individuals, each allocated to receive a single Compound 1 IVT Implant (70 microgram (g) of dexamethasone in a 0.074 microliter (L) implant) at baseline.

### Group 2

The Group 2 treatment group consists of about 27 individuals, each allocated to receive two Compound 1 IVT Implants (140 g of dexamethasone in two 0.074 μL implants) at baseline.

### Data Collection

In some instances, the data is calculated as an average aggregate for the Group 1 (1 implant of Compound 1 (containing 92 μg+/−15% of Compound 1)) and Group 2 (2 implants of Compound 1 (each implant containing 92 μg+/−15% of Compound 1) RVO and/or DME cohorts. In some instances, a mean change in ETDRS letters is provided relative to the BCVA recorded at the baseline visit prior to treatment administration. In some instances, average aggregate data includes individuals up to time of rescue, such as, where the number of individuals at each timepoint may decrease if an individual receives rescue therapy before or at a respective timepoint the study. In some instances, a significant efficacy difference between the Group 1 and Group 2 cohorts is not clear (e.g., the different number of individuals in each cohort at each timepoint can be considered when comparing the data). In some instances, fluctuations in data are attributed to high-performing patients (gain of >20 letters) and/or rescued (e.g., letter loss) individual.

### Protocol

For both groups, diagnosis is assessed for both eyes at screening as part of each individual's eligibility assessment. Where both eyes are eligible, the eye with the shorter duration of macular edema is selected as the study eye.

Eligibility and all site-related treatment decisions is made based on Investigator assessment of best-corrected visual acuity (BCVA) and reading center assessment of Optical Coherence Tomography (OCT). All OCT, fluorescein angiography images and fundus photographs are reviewed by a qualified Central Reading Center (CRC). The efficacy outcomes pertaining to central subfield thickness (CST) are determined by the OCT assessments from the CRC. Lens opacities are assessed by the Investigator using the Lens Opacity Classification System III (LOCS III) grading system.

Study individuals who are lost to follow-up prior to the 1-week study visit are replaced at the Sponsor's discretion.

All individuals are followed for a planned evaluation period of 18 months. Follow-up visits are performed at weeks 1 and 4 and monthly thereafter through 12 months followed by assessment visits at 15- and 18-months post-baseline. All visits include BCVA and Optical Coherence Tomography for Central Subfield Thickness (OCT-CST).

What is claimed is:

1. A method for improving vision (e.g., visual acuity) in an individual in need thereof, the method comprising (e.g., intravitreally) administering to an eye of the individual in need thereof one or more implant, each implant comprising a compound having a structure represented by Formula (I): 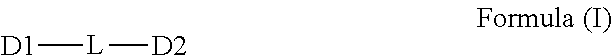
or a pharmaceutically acceptable salt or solvate thereof,
wherein,
D1 and D2 are each independently a steroid radical (e.g., a dexamethasone radical); and
L is a (e.g., hydrolyzable) linker, and
wherein the one or more implant collectively comprises an amount of the compound sufficient to improve vision in the eye of the individual in need thereof.

2. The method of claim 1, wherein the vision (e.g., the visual acuity) of the individual improves within about two months (e.g., within about one month, within about two weeks, or within about one week) of intravitreally administering the implant to the eye of the individual in need thereof.

3. The method of claim 1 or 2, wherein an improvement in vision is measured by a first (e.g., baseline) visual acuity measurement (e.g., from an ETDRS letter test) and a second (e.g., subsequent) visual acuity measurement (e.g., from an ETDRS letter test) (e.g., wherein the second visual acuity measurement increases compared to the first (e.g., baseline) visual acuity measurement).

4. The method of claim 3, wherein the second visual acuity measurement is obtained within about 12 months (e.g., within about 11 months, within about 10 months, within about 9 months, within about 8 months, within about 7 months, within about 6 months, within about 5 months, within about 4 months, within about 3 months, within about 2 months, within about 1 month) of the first visual acuity measurement.

5. The method of claim 3 or claim 4, wherein the first visual acuity measurement and the second visual acuity measurement are measured by Best Corrected Visual Acuity (BCVA).

6. The method of any one of claims 3 to 5, wherein the vision of the individual (e.g., as measured by one or more ETDRS letter test) is improved by at least 5 letters (e.g., 5 or more letters, 6 or more letters, 7 or more letters, 8 or more letters, 9 or more letters, 10 or more letters, 11 or more letters, 15 or more letters, 20 or more letters, 25 or more letters, or 30 or more letters) by BCVA.

7. The method of any one of the preceding claims, wherein the vision of the individual remains improved (e.g., a second visual acuity measurement remains higher than a first (e.g., baseline) visual acuity measurement) for at least ab out 2 months (e.g., about 2 months or more, about 3 months or more, about 4 months or more, about 5 months or more, about 6 months or more, about 7 months or more, about 8 months or more, about 9 months or more, about 10 months or more, about 11 months or more, or about 12 months or more) after intravitreally administering the implant to the eye of the individual in need thereof.

8. A method for treating vision deterioration in an individual in need thereof, the method comprising (e.g., intravitreally) administering to an eye of the individual in need thereof one or more implant, each implant comprising a compound having a structure represented by Formula (I): 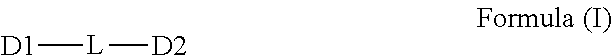
or a pharmaceutically acceptable salt or solvate thereof,
wherein,
D1 and D2 are each independently a steroid radical (e.g., a dexamethasone radical); and
L is a (e.g., hydrolyzable) linker, and
wherein the one or more implant collectively comprises an amount of the compound sufficient to treat the vision deterioration in the eye of the individual in need thereof.

9. The method of claim 8, wherein treating vision deterioration comprises reducing a rate of vision deterioration, maintaining a (current) level of vision, and/or improving vision in the individual in need thereof.

10. A method for treating an ocular disorder or condition in an individual in need thereof, the method comprising (e.g., intravitreally) administering to an eye of the individual in need thereof a first implant and a second implant, the second implant being administered no less than one month (e.g., one month or more, two months or more, three months or more, four months or more, five months or more, six months or more, seven months or more, eight months or more, nine months or more, ten months or more, 11 months or more, or 12 months or more) after the first implant, each implant comprising a compound having a structure represented by Formula (I): 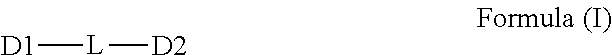
or a pharmaceutically acceptable salt or solvate thereof,
wherein,
D1 and D2 are each independently a steroid radical (e.g., a dexamethasone radical); and
L is a (e.g., hydrolyzable) linker, and
wherein the one or more implant collectively comprises an amount of the compound sufficient to treat the ocular disorder or condition in the eye of the individual in need thereof.

11. The method of any one of the preceding claims, wherein a third implant is administered to the eye of the individual in need thereof.

12. The method of any one of the preceding claims, wherein the second implant is administered within about three months of the first implant being administered to the eye of the individual in need thereof.

13. The method of any one of the preceding claims, wherein the third implant is administered to the eye of the individual in need thereof about six months or more after the first implant is administered to the eye of the individual in need thereof.

14. The method of any one of the preceding claims, wherein the third implant is administered to the eye of the individual in need thereof about three months or more after the second implant is administered to the eye of the individual in need thereof.

15. The method of any one of the preceding claims, wherein the second implant is administered to the eye of the individual in need thereof at least six months after the first implant is administered to the eye of the individual in need thereof.

16. The method of claims 1-15, wherein the second implant is administered to the eye of the individual in need thereof no more than six months after the first implant is administered to the eye of the individual in need thereof (e.g., wherein the individual is lacking a response to the first implant, such as, lacking a decrease in CST of 5% or more compared to baseline, such as at or before month 3).

17. A method for treating an ocular disorder or condition in an individual in need thereof, the method comprising (e.g., intravitreally) administering to an eye of the individual in need thereof one or more implant no more frequently than once every three months, each implant comprising a compound having a structure represented by Formula (I): 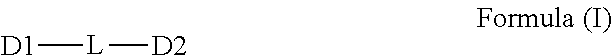
or a pharmaceutically acceptable salt or solvate thereof,
wherein,
D1 and D2 are each independently a steroid radical (e.g., a dexamethasone radical); and
L is a (e.g., hydrolyzable) linker, and
wherein the one or more implant collectively comprises an amount of the compound sufficient to treat the ocular disorder or condition in the eye of the individual in need thereof.

18. A method for treating an ocular disorder or condition in an individual in need thereof, the method comprising:
(i) administering (e.g., intravitreally) to an eye of the individual in need thereof a (pharmaceutical composition comprising a) first agent (e.g., an anti-inflammatory agent and/or an angiogenesis inhibitor, such as an anti-growth factor and/or an anti-angiogenic factor); and
(ii) administering (e.g., intravitreally) to the eye of the individual in need thereof one or more implant comprising a compound having a structure represented by Formula (I): 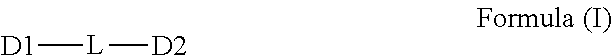
or a pharmaceutically acceptable salt or solvate thereof,
wherein,
D1 and D2 are each independently a steroid radical (e.g., a dexamethasone radical); and
L is a (e.g., hydrolyzable) linker.

19. The method of claim 18, wherein the first agent and the one or more implant are administered to the eye of the individual sequentially.

20. The method according to claim 18 or 19, wherein the one or more implant is administered to the eye of the individual (e.g., at least one week) after the first agent is administered to the eye of the individual.

21. The method of any one of claims 18-20, wherein the first agent is a vascular endothelial growth factor (VEGF) treatment (e.g., an anti-VEGF treatment), such as an anti-VEGF injection.

22. The method of any one of claims 18-21, wherein the first agent is a VEGF inhibitor.

23. The method of any one of claims 18-22, wherein the first agent is an anti-vascular endothelial growth factor-A (anti-VEGF-A).

24. The method of any one of claims 18-23, further comprising injecting an (e.g., intravitreal (IVT)) injection comprising the first agent (e.g., a steroid or an angiogenesis inhibitor) into the eye of the individual in need thereof.

25. The method of any one of claims 18-24, wherein the one or more implant is administered after the first agent to extend treatment (e.g., (to maintain a) reduction of symptoms, improvement in visual acuity (e.g., as measured by BCVA), and/or reduction in CST) in the eye of the individual, such as for about one month or more (e.g., 6 months or more or 12 months or more).

26. The method of any one of claims 17-25, wherein the ocular disorder or condition is a posterior ocular disease or disorder.

27. The method of any one of claims 17-26, wherein the ocular disorder or condition is a macular edema.

28. The method of any one of claims 17-27, wherein the ocular disorder or condition is a retinal disorder.

29. The method of any one of claims 17-28, wherein the ocular disorder or condition is diabetic macular edema (DME), macular edema secondary to retinal vein occlusion (RVO), or uveitis.

30. The method of any one of claims 17-29, wherein the ocular disorder or condition is DME or RVO.

31. The method of any one of claims 17-30, wherein the ocular disorder or condition is DME.

32. The method of any one of claims 17-30, wherein the ocular disorder or condition is RVO (e.g., central RVO).

33. The method of any one of the preceding claims, wherein the implant is administered to the individual in need thereof once every three months or less, once every 4 months or less, once every 5 months or less, or once every 6 months or less.

34. The method of any one of the preceding claims, wherein the implant is administered to the individual in need thereof once every three to twelve months (e.g., once every six months).

35. A method for decreasing central subfield thickness (CST) in an individual in need thereof, the method comprising (e.g., intravitreally) administering to an eye of the individual in need thereof one or more implant, each implant comprising a compound having a structure represented by Formula (I): 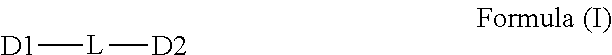
or a pharmaceutically acceptable salt or solvate thereof,
wherein,
D1 and D2 are each independently a steroid radical (e.g., a dexamethasone radical); and
L is a (e.g., hydrolyzable) linker, and
wherein the one or more implant collectively comprises an amount of the compound sufficient to decrease CST in the eye of the individual in need thereof.

36. The method of any one of the preceding claims, wherein CST in the eye of the individual in need thereof decreases by at least 50 micrometers (μm) (e.g., 50 μm or more, 100 μm or more, 150 μm or more, or 200 μm or more), such as within about one month (e.g., within about one week) of intravitreally administering the implant to the eye of the individual in need thereof.

37. The method of any one of the preceding claims, wherein CST in the eye of the individual in need thereof decreases by at least 200 μm over an extended period of time, such as for a month or more (e.g., 6 months or more) after intravitreally administering the implant to the eye of the individual in need thereof.

38. The method of any one of the preceding claims, wherein CST in the eye of the individual in need thereof decreases by at least 10% (e.g., 10% or more, 15% or more, 20% or more, 25% or more, 30% or more, 35% or more, or 40% or more) of the baseline CST, such as over an extended period of time, such as for a month or more (e.g., 6 months or more) after intravitreally administering the implant to the eye of the individual in need thereof.

39. The method of any one of the preceding claims, wherein a decrease in CST is measured by a first (e.g., baseline) CST measurement and a second (e.g., subsequent) CST measurement (e.g., wherein the second CST measurement decreases compared to the first (e.g., baseline) CST measurement).

40. The method of any one of the preceding claims, wherein CST in the eye of the individual decreases within about three months (e.g., within about two months, within about one month, within about two weeks, or within about one week) of intravitreally administering the implant to the eye of the individual in need thereof.

41. The method of any one of the preceding claims, wherein CST of the individual remains decreased (e.g., a second CST measurement remains lower than a first (e.g., baseline) CST measurement) for at least about 2 months (e.g., about 2 months or more, about 3 months or more, about 4 months or more, about 5 months or more, about 6 months or more, about 7 months or more, about 8 months or more, about 9 months or more, about 10 months or more, about 11 months or more, or about 12 months or more) after intravitreally administering the implant to the eye of the individual in need thereof.

42. The method of any one of the preceding claims, wherein one or more implant is (e.g., intravitreally) administered to one or more eye of each individual of a population of individuals.

43. The method of any one of the preceding claims, wherein the population of individuals have a mean change in CST that decreases (e.g., a second mean change in CST measurement for the population at a certain timepoint (e.g., at one-month of treatment) decreases compared to a first (mean change) in CST measurement for the population at another timepoint (e.g., baseline)), such as over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

44. The method of any one of the preceding claims, wherein the mean change in CST decreases by about 50 μm or more (e.g., by about 100 μm or more, about 150 μm or more, about 200 μm or more, about 250 μm or more), such as over an extended period of time, such as a month or more (e.g., 6 months or more) after intravitreally administering the implant to the eye of each individual of the population of individuals.

45. The method of any one of the preceding claims, wherein the mean change in CST decreases by at least about 10% (e.g., about 10% or more, about 15% or more, about 20% or more, about 25% or more, about 30% or more, about 35% or more, about 40% or more, about 50% or more, about 60% or more, about 70% or more, or about 80% or more), such as compared to a baseline CST measurement.

46. The method of any one of the preceding claims, wherein a population of individuals with diabetic macular edema (DME) is administered two implants and the population has a mean change in CST that decreases, such as by at least about 60 μm or more (e.g., about 80 μm or more, about 100 μm or more, about 120 μm or more), over an extended period of time, such as over a period of at least about 1 month or more (e.g., about 2 months or more, about 3 months or more, about 4 months or more, about 5 months or more).

47. The method of any one of claims 1-45, wherein a population of individuals with diabetic macular edema (DME) is administered one implant and the population has a mean change in CST that decreases, such as by at least about 40 μm or more (e.g., about 60 μm or more, about 80 μm or more, about 100 μm or more), over an extended period of time, such as over a period of at least about 1 month or more (e.g., about 2 months or more, about 3 months or more, about 4 months or more, about 5 months or more).

48. The method of any one of claims 1-45, wherein a population of individuals with retinal vein occlusion (RVO) is administered two implants and the population has a mean change in CST that decreases, such as by at least about 100 μm or more (e.g., about 150 μm or more, about 200 μm or more), over an extended period of time, such as over a period of at least about 1 month or more (e.g., about 2 months or more, about 3 months or more, about 4 months or more, about 5 months or more, about 6 months or more).

49. The method of any one of claims 1-45, wherein a population of individuals with retinal vein occlusion (RVO) is administered one implant and the population has a mean change in CST that decreases, such as by at least about 150 μm or more (e.g., about 200 μm or more, about 250 μm or more), over an extended period of time, such as over a period of at least about 1 month or more (e.g., about 2 months or more, about 3 months or more, about 4 months or more, about 5 months or more, about 6 months or more).

50. The method of any one of the preceding claims, wherein the population of individuals have a mean change in vision (improvement) (e.g., as measured by one or more ETDRS letter test) that increases (e.g., a second mean change in vision (improvement) measurement for the population at a certain timepoint (e.g., at one month of treatment) increases compared to a first mean change in vision (improvement) measurement for the population at another timepoint (e.g., baseline)), such as over a period of about one week (e.g., about 1 month or more, about 2 months or more, about 3 months or more, about 4 months or more, about 5 months or more, about 6 months or more).

51. The method of any one of the preceding claims, wherein the vision of the population of individuals (e.g., as measured by one or more ETDRS letter test) improves, such as by at least 5 letters (e.g., 5 or more letter, 6 or more letters, 7 or more letters, 8 or more letters, 9 or more letters) by BCVA.

52. The method of any one of the preceding claims, wherein a population of individuals with RVO or DME is administered one or two implants and the population has a mean change in vision (improvement) of at least 5 letters by BCVA, such as over a period of at least about 1 month or more (e.g., about 2 months or more, about 3 months or more, about 4 months or more, about 5 months or more, about 6 months or more, about 7 months or more, about 8 months or more, about 9 months or more, about 10 months or more, about 11 months or more, about 12 months or more).

53. The method of any one of the preceding claims, wherein either or both of D1 or D2 are attached to L through an oxygen radical of the D1 or D2 (e.g., thereby forming a C—O bond).

54. The method of any one of the preceding claims, wherein the compound has a structure represented by Formula (I-A): 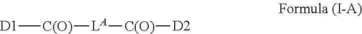
or a pharmaceutically acceptable salt or solvate thereof,
wherein,
D1 and D2 are each independently a steroid radical (e.g., a dexamethasone radical); and
L^{A} is substituted or unsubstituted alkyl or substituted or unsubstituted heteroalkyl (e.g., comprising one or more ethyleneglycol unit).

55. The method of any one of the preceding claims, wherein an oxygen radical of the D1 and an oxygen radical of the D2 are each independently attached to a C(O) (e.g., the C(O) of Formula (I-A) is attached to D1 and D2, respectively).

56. The method of any one of the preceding claims, wherein L^{A} is unsubstituted heteroalkyl.

57. The method of any one of the preceding claims, wherein L^{A} is —O(CH₂CH₂O)ₙ, where n is an integer of 1-10.

58. The method of any one of the preceding claims, wherein n is an integer of 3-5.

59. The method of any one of the preceding claims, wherein n is 3.

60. The method of any one of the preceding claims, wherein D1 and D2 are each represented by the same steroid radical structure.

61. The method of any one of the preceding claims, wherein D1 and D2 are each a dexamethasone radical (e.g., each independently being connected to the linker (e.g., L or L^{A}) by an oxygen radical, thereby forming a C—O bond).

62. The method of any one of the preceding claims, wherein the compound has a structure represented by:

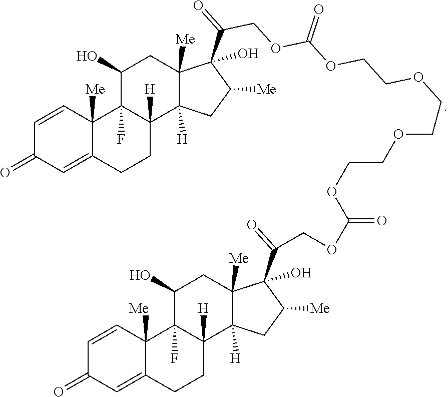

63. The method of any one of the preceding claims, wherein the implant comprises no more than about 500 micrograms (μg) of the compound.

64. The method of any one of the preceding claims, wherein the implant comprises about 10 μg or more of the compound.

65. The method of any one of the preceding claims, wherein the implant comprises about 10 μg to about 500 μg of the compound.

66. The method of any one of the preceding claims, wherein the implant comprises about 250 μg or less of the compound.

67. The method of any one of the preceding claims, wherein the implant comprises about 156 μg or less of the compound.

68. The method of any one of the preceding claims, wherein the implant comprises about 50 μg or more of the compound.

69. The method of any one of the preceding claims, wherein the implant comprises about 75 μg or more of the compound.

70. The method of any one of the preceding claims, wherein the implant comprises about 50 μg to about 250 μg of the compound.

71. The method of any one of the preceding claims, wherein the implant comprises about 75 μg to about 175 μg of the compound.

72. The method of any one of the preceding claims, wherein the implant comprises about 78 μg to about 106 μg of the compound.

73. The method of any one of the preceding claims, wherein intraocular pressure (IOP) of the eye of the individual in need thereof does not increase above a clinically acceptable level (e.g., while the compound is released from the implant in the eye of the individual).

74. The method of any one of the preceding claims, wherein intraocular pressure (IOP) of the eye of the individual in need thereof remains at a clinically acceptable level (e.g., while the compound is released from the implant in the eye of the individual).

75. The method of any one of the preceding claims, wherein one or more implant is (e.g., intravitreally) administered to one or more eye of each individual of a population of individuals, wherein the population of individuals have a change in IOP of no more than about 15 millimeters of mercury (mmHg) (e.g., while the compound is released from the one or more implant in the one or more eye), such as compared to a baseline measurement and/or over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

76. The method of any one of the preceding claims, wherein a population of individuals with RVO is administered one or two implants and the population of individuals have a change in IOP of no more than about 10 mmHg (e.g., while the compounds released from the one or more implant in the one or more eye), such as compared to a baseline measurement and/or over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

77. The method of any one of the preceding claims, wherein a population of individuals with DME is administered one or two implants and the population of individuals have a change in IOP of no more than about 5 mmHg (e.g., while the compound is released from the one or more implant in the one or more eye), such as compared to a baseline measurement and/or over a period of at least about one week (e.g., about 1 month or more, about 2 months or more, about 3 months or more, about 4 months or more, about 5 months or more, about 6 months or more, about 7 months or more, about 8 months or more, about 9 months or more, about 10 months or more, about 11 months or more, or about 12 months or more).

78. The method of any one of the preceding claims, wherein IOP of an eye of an individual of the population of individuals does not increase above a level of about 35 mmHg (e.g., while the compound is released from the implant in the eye of the individual).

79. The method of any one of the preceding claims, wherein IOP of the eye of the individual in need thereof does not increase above a level of about 25 millimeters of mercury (mmHg) (e.g., while the compound is released from the implant in the eye of the individual).

80. The method of any one of the preceding claims, wherein IOP of the eye of the individual in need thereof remains below a level of about 25 mmHg (e.g., while the compound is released from the implant in the eye of the individual).

81. The method of any one of the preceding claims, wherein the amount of the compound administered to the individual in need thereof is sufficient to improve vision, treat vision deterioration, treat an ocular disorder or condition, and/or decrease CST in the eye of the individual in need thereof while avoiding an increase (e.g., a clinically significant increase) in intraocular pressure (IOP) in the individual in need thereof.

82. The method of any one of the preceding claims, wherein the amount of the compound sufficient to improve vision, treat vision deterioration, treat an ocular disorder or condition, and/or decrease CST in the eye of the individual in need thereof is less than about 500 micrograms (μg) (e.g., 500 μg or less, 400 μg or less, 300 μg or less, 200 μg or less, 100 μg or less, or 50 μg or less).

83. The method of any one of the preceding claims, wherein the amount of the compound sufficient to improve vision, treat vision deterioration, treat an ocular disorder or condition, and/or decrease CST in the eye of the individual in need thereof is less than about 250 μg.

84. The method of any one of the preceding claims, wherein the amount of the compound sufficient to improve vision, treat vision deterioration, treat an ocular disorder or condition, and/or decrease CST in the eye of the individual in need thereof is more than about 10 μg.

85. The method of any one of the preceding claims, wherein the amount of the compound sufficient to improve vision, treat vision deterioration, treat an ocular disorder or condition, and/or decrease CST in the eye of the individual in need thereof is about 10 μg to about 500 μg (e.g., about 50 μg to about 250 μg).

86. The method of any one of the preceding claims, wherein the amount of the compound sufficient to improve vision, treat vision deterioration, treat an ocular disorder or condition, and/or decrease CST in the eye of the individual in need thereof is about 50 μg to about 250 μg.

87. The method of any one of the preceding claims, wherein the amount of the compound sufficient to improve vision, treat vision deterioration, treat an ocular disorder or condition, and/or decrease CST in the eye of the individual in need thereof is less than about 175 μg.

88. The method of any one of the preceding claims, wherein the amount of the compound sufficient to improve vision, treat vision deterioration, treat an ocular disorder or condition, and/or decrease CST in the eye of the individual in need thereof is less than about 156 μg (e.g., less than about 100 μg).

89. The method of any one of the preceding claims, wherein the amount of the compound sufficient to improve vision, treat vision deterioration, treat an ocular disorder or condition, and/or decrease CST in the eye of the individual in need thereof is about 75 μg to about 175 μg.

90. The method of any one of the preceding claims, wherein the amount of the compound sufficient to improve vision, treat vision deterioration, treat an ocular disorder or condition, and/or decrease CST in the eye of the individual in need thereof is about 50 μg to about 110 μg (e.g., about 78 μg to about 106 μg).

91. The method of any one of the preceding claims, wherein the implant is intravitreally administered to the eye of the individual in need thereof in an amount of about 10 μg to about 500 μg of the compound.

92. The method of any one of the preceding claims, wherein the implant is intravitreally administered to the eye of the individual in need thereof in an amount of about 250 μg or less of the compound.

93. The method of any one of the preceding claims, wherein the implant is intravitreally administered to the eye of the individual in need thereof in an amount of about 100 μg or less of the compound.

94. The method of any one of the preceding claims, wherein the implant is intravitreally administered to the eye of the individual in need thereof in an amount of less than about 175 μg of the compound.

95. The method of any one of the preceding claims, wherein the implant is intravitreally administered to the eye of the individual in need thereof in an amount of about 75 μg to about 175 μg of the compound.

96. The method of any one of the preceding claims, wherein the implant is intravitreally administered to the eye of the individual in need thereof in an amount of about 78 μg to about 106 μg of the compound.

97. The method of any one of the preceding claims, wherein another implant is (e.g., intravitreally) administered to the eye of the individual in need thereof, such as before the one or more implant is completed degraded (e.g., about 35% of the one or more implant by mass remains).

98. The method of any one of the preceding claims, wherein the implant comprises at least 70% weight by weight (w/w) (e.g., 70% w/w or more, 80% w/w or more, 90% w/w or more, 95% w/w or more, or 99% w/w or more) of the compound.

99. The method of any one of the preceding claims, wherein the implant comprises at least 95% w/w (e.g., 95% w/w or more, 96% w/w or more, 97% w/w or more, 98% w/w or more, or 99% w/w or more) of the compound.

100. The method of any one of the preceding claims, wherein the implant is a fiber, a fiber mesh, a woven fabric, a non-woven fabric, a pellet, a cylinder (e.g., a cylinder, a rod, or a cylindrical-shaped implant), a hollow tube, a microparticle, a nanoparticle, or a shaped article.

101. The method of any one of the preceding claims, wherein the implant is a cylinder.

102. The method of any one of the preceding claims, wherein the implant releases the compound or dexamethasone in its free form in an amount sufficient to improve vision, treat vision deterioration, treat an ocular disorder or condition, and/or decrease CST in the eye of the individual in need thereof.

103. The method of any one of the preceding claims, wherein the implant releases the compound or dexamethasone in its free form over a period of at least a day.

104. The method of any one of the preceding claims, wherein the implant releases the compound or dexamethasone in its free form over a period of at least a week.

105. The method of any one of the preceding claims, wherein the implant releases the compound or dexamethasone in its free form over a period of at least a month.

106. The method of any one of the preceding claims, wherein the implant releases the compound or dexamethasone in its free form over a period of 6 months or more.

107. The method of any one of the preceding claims, wherein the individual in need thereof has been diagnosed with a posterior ocular disease or disorder.

108. The method of any one of the preceding claims, wherein the individual in need thereof has been diagnosed with a macular edema.

109. The method of any one of the preceding claims, wherein the individual in need thereof has been diagnosed with a retinal disorder.

110. The method of any one of the preceding claims, wherein the retinal disorder is a retinal vein occlusion (RVO).

111. The method of any one of the preceding claims, wherein the individual in need thereof has been diagnosed with diabetic macular edema (DME), macular edema secondary to retinal vein occlusion (RVO), or uveitis.

112. The method of any one of the preceding claims, wherein the individual in need thereof has been diagnosed with DME or RVO.

113. The method of any one of the preceding claims, wherein the individual in need thereof has been diagnosed with DME.

114. The method of any one of the preceding claims, wherein the individual in need thereof has been diagnosed with RVO (e.g., central RVO).

115. The method of any one of the preceding claims, wherein the individual in need thereof has a deterioration of vision.

116. The method of any one of the preceding claims, wherein the deterioration of vision is perceived by the individual and/or measured, such as, by a subjective method (e.g., vision test).

117. Use of an implant of any one of the preceding claims for manufacture of a medicament for use in a method of any one of the preceding claims.

118. A composition comprising the compound of any one of the preceding claims in an amount of about 250 micrograms (μg) or less, wherein the composition comprises at least 90% w/w of the compound.

119. An implant comprising the compound of any one of the preceding claims in an amount of about 250 micrograms (μg) or less, wherein the implant comprises at least 90% w/w of the compound.

120. A dosage form comprising the compound of any one of the preceding claims in an amount of about 250 micrograms (μg) or less, wherein the dosage form comprises at least 90% w/w of the compound.

121. A kit comprising (i) an (e.g., intravitreal) injector, and (ii) no more than two and no less than one implant, the implant comprising the compound of any one of the preceding claims in an amount of about 250 micrograms (μg) or less, wherein the implant comprises at least 90% w/w of the compound.

122. The kit of claim 121, wherein one implant is pre-loaded into the (e.g., intravitreal) injector.

123. The kit of claim 121, wherein two implants are pre-loaded into the (e.g., intravitreal) injector.

124. The composition, implant, dosage form, or kit of any one of the preceding claims, wherein the composition or implant comprises no more than about 175 μg of the compound of any one of the preceding claims.

125. The composition, implant, dosage form, or kit of any one of the preceding claims, wherein the composition or implant comprises about 50 μg to about 150 μg of the compound of any one of the preceding claims.

126. The composition, implant, dosage form, or kit of any one of the preceding claims, wherein the composition or implant comprises about 50 μg to about 110 μg (e.g., 78 μg to about 106 μg) of the compound of any one of the preceding claims.

127. The composition, implant, dosage form, or kit of any one of the preceding claims, wherein the compound has a structure represented by:

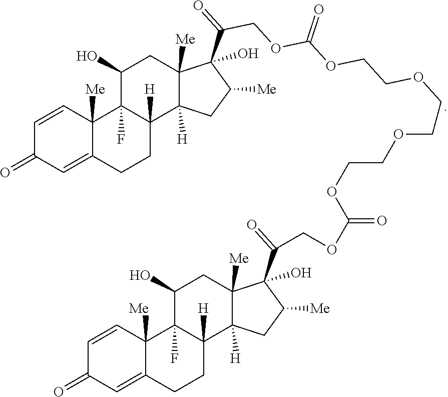

128. The composition, implant, dosage form, or kit of any one of the preceding claims, wherein the composition or implant comprises least 95% w/w (e.g., 95% w/w or more, 96% w/w or more, 97% w/w or more, 98% w/w or more, or 99% w/w or more) of the compound of any one of the preceding claims.
